(19) United States
(12) Patent Application Publication
(10) Pub. No.: US 2025/0144249 A1
(43) Pub. Date: May 8, 2025

(54) METHODS USING SURFACE-EXPRESSIBLE ACTIVATABLE EPITOPES TO LOCALIZE AND/OR TREAT DISEASED CELLS

(71) Applicant: EARLI Inc., South San Francisco, CA (US)

(72) Inventors: Alex HARWIG, Redwood City, CA (US); Evan BISHOP, Redwood City, CA (US); AMANDA KOHLER, Redwood City, CA (US); David SUHY, Redwood City, CA (US)

(21) Appl. No.: 18/936,497

(22) Filed: Nov. 4, 2024

Related U.S. Application Data
(63) Continuation of application No. PCT/US2023/020892, filed on May 3, 2023.
(60) Provisional application No. 63/429,783, filed on Dec. 2, 2022.
(60) Provisional application No. 63/338,435, filed on May 4, 2022.

(51) Int. Cl. A61K 48/00; A61K 38/17; A61K 47/64; A61K 49/14; A61K 51/08; C12N 15/85

(52) U.S. Cl. CPC A61K 48/0066; A61K 38/1709; A61K 47/64; A61K 49/14; A61K 51/08; C12N 15/85; A61K 2123/00; C12N 2800/00

(57) ABSTRACT

Biomarkers, affinity-based reagents, and methods of using such to detect, image, or treat cancer are described herein.

### CROSS-REFERENCE

This application is a continuation of PCT/US2023/020892, filed May 3, 2023, which claims the benefit of U.S. Provisional Application No. 63/338,435, filed on May 4, 2022, entitled “METHODS USING SURFACE-EXPRESSIBLE ACTIVATABLE EPITOPES TO IMAGE AND/OR TREAT DISEASE CELLS” and U.S. Provisional Application No. 63/429,783, filed on Dec. 2, 2022, entitled “METHODS USING SURFACE-EXPRESSIBLE ACTIVATABLE EPITOPES TO LOCALIZE AND/OR TREAT DISEASED CELLS”, each of which are incorporated by reference herein in their entireties.

### SEQUENCE LISTING

The instant application contains a Sequence Listing which has been submitted electronically in XML format and is hereby incorporated by reference in its entirety. Said XML format Sequence Listing, created on Jan. 3, 2025, is named 53531_711_301_SL.xml and is 114,797 bytes in size.

### BACKGROUND

Imaging modalities such as immuno-PET allow the specificity of antibodies and protein ligands to be combined with the sensitivity of PET imaging.

Additionally, affinity-based reagents, antibodies and protein ligands that have been conjugated with radionuclide moieties, toxins and peptide fragments can be used are used to induce cellular damage and cause death of cells coincident with PET imaging using antibodies and protein ligands.

### SUMMARY

A combination of immuno-PET (which uses affinity-based reagents) with affinity-based reagents conjugated with detectable or therapeutic moieties (radionuclide moieties, toxins and peptide fragments that are used to induce cellular damage) and induced cancer-specific expression of markers targeted by the affinity-based reagents can allow for convenient new modalities for detecting, imaging or treating cancer in situ. Described herein are methods, systems, and compositions for enhancing affinity-based targeting of cancers for detection or treatment.

In some aspects, the present disclosure provides for a nucleic acid encoding an engineered polypeptide, comprising: (a) an extracellularly-oriented domain comprising an epitope capable of binding: (i) an antibody or (ii) a peptide hormone or growth factor, wherein the extracellularly-oriented domain does not comprise an scFv; (b) an extracellularly-oriented polypeptide spacer domain of about 15 to about 40 angstroms in length when folded; and (c) a transmembrane domain or a membrane affinity domain capable of associating with an outer membrane of a cell, wherein at least two of (a), (b), and (c) are heterologous to each other. In some embodiments, the polypeptide does not comprise a light chain variable (VL) domain. In some embodiments, the engineered polypeptide does not comprise an intracellular signaling domain. In some embodiments, the engineered polypeptide does not comprise an intracellular portion of a CD3zeta, CD137, or CD28 polypeptide. In some embodiments, an intracellularly-oriented portion of the polypeptide comprises less than or equal to 100, 75, 50, 25, 10, or 5 residues. In some embodiments, the extracellularly-oriented polypeptide spacer domain comprises a hinge domain. In some embodiments, (a), (b), and (c) are in order from N- to C-terminus of the engineered polypeptide. In some embodiments, the engineered polypeptide is capable of display on a cell surface. In some embodiments, wherein the epitope further comprises an activatable epitope selectively available for binding an extracellular ligand in a tumor microenvironment. In some embodiments, the activatable epitope is flanked by at least two copies of pH-sensitive helices. In some embodiments, the epitope capable of binding: (i) the antibody or (ii) the peptide hormone or growth factor is derived from DLL3, PSMA, SSTR2, or any combination thereof. In some embodiments, the epitope is derived from DLL3, wherein the epitope comprises about 15 to about 260 contiguous residues of an extracellular domain of DLL3 having at least 80% identity to SEQ ID NO: 5 or an extracellular domain of any of the proteins described herein, or a variant thereof. In some embodiments, the epitope comprises a sequence having at least 80% sequence identity to any one of SEQ ID NOs: 4, 5, 19, 20, 64, 65, or a variant thereof. In some embodiments, the epitope is capable of binding: (i) the antibody or (ii) the peptide hormone or growth factor with a Kd of less than or equal to 20 nM, 15 nM, 10 nM, 9 nM, 8 nM, 7 nM, 6 nM, 5 nM, 4 nM, 3 nM, 2 nM, or 1 nM. In some embodiments, the extracellularly-oriented polypeptide spacer domain comprises a hinge sequence from CD8, CD8a, CD8b, IgG4, IgG1, IgG2, IgG3, IgK, CD4, or CD28, or any combination thereof. In some embodiments, the extracellularly-oriented polypeptide spacer domain comprises a hinge sequence having at least 80% sequence identity to a hinge sequence of any one of SEQ ID NOs: 56, 57, 58, 59, 60, 61, 62, 66, 67, or hinge sequences of any of the proteins described herein, or a variant thereof. In some embodiments, the transmembrane domain or the membrane affinity domain comprises a transmembrane domain. In some embodiments, the transmembrane domain comprises a single-pass transmembrane domain. In some embodiments, the transmembrane domain comprises a transmembrane domain derived from DLL3, PSMA, SSTR2, PD-L1, EGFR, CD28, CD4, CD8, CD8a, CD8b, ICOS, or CD73. In some embodiments, the transmembrane domain comprises a sequence having at least 80% sequence identity to a transmembrane domain of any one of SEQ ID NOs: 68-76, or a transmembrane domain of any of the proteins described herein, or a variant thereof. In some embodiments, the transmembrane domain or the membrane affinity domain comprises a membrane affinity domain. In some embodiments, the membrane affinity domain comprises a C1, C2, PH, FYVE, PX, or ENTH domain, or a transmembrane helix derived from bacteriorhodopsin. In some embodiments, the membrane affinity domain comprises a sequence having at least 80% sequence identity to any one of SEQ ID NOs: 77-82, or to a membrane affinity domain of any of the proteins described herein, or a variant thereof. In some embodiments, the engineered polypeptide further comprises a scaffold domain: (i) N-terminal to the hinge domain and C-terminal to the epitope; or (ii) N-terminal to the hinge domain and containing the epitope. In some embodiments, the engineered polypeptide further comprises a scaffold domain N-terminal to the hinge domain and C-terminal to the epitope. In some embodiments, wherein the scaffold comprises a sequence having at least 80% sequence identity to SEQ ID NO: 63, or a variant thereof. In some embodiments, wherein the engineered polypeptide further comprises a scaffold domain N-terminal to the hinge domain and containing the epitope. In some embodiments, the scaffold comprises a heavy chain variable (VH) domain and does not comprise a light chain variable (VL) domain. In some embodiments, the VH domain comprises an inactivating mutation in a CDR1, CDR2, or CDR3 region of the VH domain. In some embodiments, the scaffold comprises the epitope inserted in a CDR1, CDR2, or CDR3 region of the VH domain. In some embodiments, the nucleic acid further comprises an ORF encoding the engineered polypeptide. In some embodiments, the nucleic acid further comprises a promoter operably linked to the ORF. In some embodiments, the promoter is not a T-cell specific promoter or a TCRA, TCRB, CMV, EF-1, hPGK, CD3, or RPBSA promoter. In some embodiments, the promoter is a cancer-specific promoter. In some embodiments, the promoter is a promoter of a gene overexpressed in a cancer cell versus a normal cell, or a functional fragment thereof. In some embodiments, the promoter comprises a Survivin promoter (BIRC5), a CXCR4 promoter, an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransferase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11A1) promoter, a collagen type I alpha 1 chain (COL1A1) promoter, a cystatin SN (CST1) promoter, a denticleless E3 ubiquitin protein ligase homolog (DTL) promoter, a family with sequence similarity 111 member B (FAM111B) promoter, a forkhead box A1 (FOXA1) promoter, a kinesin family member 20A (KIF20A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2A member 1 (SCGB2A1) promoter, topoisomerase II alpha (TOP2A) promoter, a ubiquitin D (UBD) promoter, a ubiquitin conjugating enzyme E2 C (UBE2C), a USH1 protein network component harmonin (USH1C), a V-set domain containing T cell activation inhibitor 1 (VTCN1) promoter, a Hexokinase type II promoter, a TRPM4 promoter, a stromelysin 3 promoter, a surfactant protein A promoter, a secretory leukoprotease inhibitor promoter, a tyrosinase promoter, a stress-inducible grp78/BiP promoter, an interleukin-10 promoter, an α-B-crystallin/heat shock protein 27 promoter, an epidermal growth factor receptor promoter, a mucin-like glycoprotein promoter, an mts1 promoter, an NSE promoter, a somatostatin receptor promoter, a c-erbB-3 promoter, a c-erbB-2 promoter, a c-erbB4 promoter, a thyroglobulin promoter, an α-fetoprotein promoter, a villin promoter, an albumin promoter, a glycoprotein A33 promoter, the B cell-specific Moloney leukemia virus insertion site 1 promoter, a cyclooxygenase-2 promoter, a fibroblast growth factor promoter; a human epidermal growth factor receptor 2, a human telomerase reverse transcriptase promoter; a kinase domain insert containing receptor promoter; a rad51 recombinase promoter; TTF-1, an urokinase-type plasminogen activator receptor promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (UBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box M1 (FOXM1) promoter, a cell proliferation regulating inhibitor of protein phosphatase 2A (KIAA1524) promoter, a kinesin family member 2C (KIF2C) promoter, a karyopherin subunit alpha 2 (KPNA2) promoter, a MYB proto-oncogene like 2 (MYBL2) promoter, a NIMA related kinase 2 (NEK2) promoter, a RAN binding protein 1 (RANBP1) promoter, a small nuclear ribonucleoprotein polypeptides B and B1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17orf53) promoter, a chromobox 3 (CBX30) promoter, a cyclin B1 (CCNB1) promoter, a cyclin E1 (CCNE1) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL10A1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18A (KIF18A) promoter, a kinesin family member 4A (KIF4A) promoter, a kinesin family member C1 (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, a kinetochore complex component (NDC80) promoter, a nudix hydrolase 1 (NUDT1) promoter, a ribonuclease H2 subunit A (RNASEH2A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP13) promoter, a trophinin associated protein (TROAP) promoter, a ubiquitin conjugating enzyme E2 C (UBE2C) promoter, a WD repeat and HMG-box DNA binding protein 1 (WDHD1) promoter, an alpha fetoprotein (AFP) promoter, a functional fragment thereof, any combination thereof, a chimeric promoter compiled from a plurality of elements from the aforementioned, or a fully synthetic promoter composed of tiled transcription-factor binding sites derived from any of the aforementioned.

In some aspects, the present disclosure provides for a vector comprising any of the nucleic acids described herein. In some embodiments, the vector is a recombinant viral vector. In some embodiments, the vector is a non-viral vector.

In some aspects, the present disclosure provides for a method of detecting, imaging, or treating a cancer cell, comprising: (a) administering to a subject a composition comprising any of the nucleic acids or vectors described herein; and (b) administering to the subject an antibody or antigen-binding fragment thereof, a protein ligand or functional fragment thereof, or a small molecule configured to bind the epitope. In some embodiments, the administering to the subject the composition comprising the nucleic acid in (a) and the administering to the subject the antibody or antigen-binding fragment thereof in (b) are separated by at least about 8, 12, 16, 24, 36, 48, 60, 72, 84, or 96 hours. In some embodiments, the composition or the antibody or antigen-binding fragment thereof, a protein ligand or functional fragment thereof, or a small molecule configured to bind the epitope are administered to the subject by parenteral, intramuscular, subcutaneous, intratumoral, rectal, vaginal, transdermal, or intravenous administration, or by cannula. In some embodiments, the composition comprising the nucleic acid or the vector is configured for intravenous administration. In some embodiments, the method further comprises detecting binding of the antibody or antigen binding fragment, protein ligand or functional fragment thereof, or small molecule to the epitope. In some embodiments, the antibody or antigen-binding fragment thereof further comprises a radioisotope or a contrast agent (e.g. an MRI or X-Ray contrast agent), or is chelated to a radioisotope or a contrast agent. In some embodiments, the antibody or antigen-binding fragment thereof further comprises a radioisotope or is chelated to a radioisotope. In some embodiments, the radioisotope comprises a positron-emitting radioisotope, an alpha-emitting radioisotope, a beta-emitting radioisotope, or a gamma-emitting radioisotope. In some embodiments, the radioisotope is a positron-emitting radioisotope and comprises ¹²⁴I, ⁶⁸Ga ¹¹C, ¹³N, ¹⁵O, ¹⁸F, ⁶⁸Ga, ⁶⁴Cu, ⁵²Mn, ⁵⁵Co, ⁸⁹Zr, ⁸²Rb, or any combination thereof. In some embodiments, the radioisotope comprises an alpha-emitting radioisotope and comprises ²²⁵Ac, ²¹¹At, ²²⁷Th, ²²⁴Ra, or any combination thereof. In some embodiments, the radioisotope comprises a beta-emitting radioisotope and comprises ¹⁷⁷Lu, ⁶⁷Cu, ¹³¹I, ⁹⁰Y, ⁸⁹Sr, ¹⁸⁶Re, ¹⁶⁵Dy, ³²P, ¹⁶⁶Ho, ¹⁸⁸Re, or any combination thereof. In some embodiments, the radioisotope is a gamma-emitting radioisotope and comprises ^{99m}Tc, ¹²³I, or ¹³¹I. In some embodiments, the antibody or antigen-binding fragment thereof further comprises a contrast agent, or is chelated to a contrast agent. In some embodiments, the contrast agent comprises an iron oxide nanoparticle (IONP), a superparamagnetic iron platinum nanoparticle, manganese (II), or gadolinium (III). In some embodiments, the antibody or antigen-binding fragment thereof further comprises an antibody drug conjugate (ADC). In some embodiments, the antibody drug conjugate is conjugated to a pyrrolobenzodiazepine (PBD), a protein toxin, diphtheria toxin, glucagon-like peptide (GLP-1), a cytotoxic immunomodulatory protein, a Fas ligand, auristatin or an analog thereof, a maytansinoid, a calicheamicin, duocarmycin or an analog thereof, or doxorubicin or an analog thereof. In some embodiments, the method further comprises detecting the binding of the antibody or antigen binding fragment, protein ligand or functional fragment thereof, or small molecule to the epitope via MRI imaging, PET imaging, SPECT imaging, photoacoustic imaging or luminescence imaging performed on the subject. In some embodiments, the epitope is displayed on a tumor cell of the subject. In some embodiments, the tumor cell is a liver, ovarian, pancreatic, breast, lung, smooth muscle, bladder, kidney, skin, prostate, or bone tumor cell.

In some aspects, the present disclosure provides for a cell comprising any of the polypeptides, nucleic acids, or vectors described herein.

In some aspects, the present disclosure provides for a composition comprising any of the polypeptides, nucleic acids, or vectors described herein and a pharmaceutically acceptable carrier. In some embodiments, the composition further comprises a transfection agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A (FIG. 1A) depicts example designs of cell-surface expressible biomarkers according to some embodiments described herein from N- to C-terminus. In some cases, the cell-surface biomarkers comprise at least an extracellular domain and a transmembrane domain. In other cases, the cell-surface biomarkers comprise signal peptides to guide membrane insertion, a hinge region, and intracellular domains.

FIG. 1B (FIG. 1B) depicts example linear construct designs from N- to C-terminus for engineered SSTR2 (top and middle) and DLL3 (bottom) constructs. pH Helix: pH-sensitive helix. Figure discloses SEQ ID NOS 88 and 91, respectively, in order of appearance.

FIG. 1C (FIG. 1C) depicts example linear construct designs from N- to C-terminus for engineered DLL3 (top) and GFP (bottom) constructs. mIgK: mouse IgK. pHLIP: pH-sensitive membrane affinity protein motif.

FIG. 2 (FIG. 2) depicts function of a cell-surface expressible biomarker containing an activatable epitope according to some embodiments described herein. In this embodiment, the activatable epitope is inserted amidst pH-sensitive helices (e.g. any of the helices described herein) such that at high pH (e.g. pH>6.0), the epitope is occluded by the co-association of the pH-sensitive helices. Upon transition of the cell-surface biomarker to low pH in a diseased microenvironment (e.g. <6.0, such as in the acidic tumor microenvironment), the co-association of the pH-sensitive helices is abolished and the epitope become accessible to binding, e.g. by antibodies or binding fragments of antibodies configured to bind the epitope. Administration of antibodies or ligands that bind the epitope can be used to detect, image, or bind the newly accessible epitope and thus assess the presence of the accessible epitope in a diseased environment.

FIG. 3 (FIG. 3) depicts function of a second cell-surface expressible biomarker containing an activatable epitope according to some embodiments described herein. In this embodiment, a natural cell surface receptor has been engineered with a cancer-activatable epitope in which the epitope sequence is inserted between a distal pH-sensitive helix (e.g. any of the helices described herein) and a proximal sequence that is only post-translationally modified once the protein reaches the cell surface in a cancer-microenvironment specific manner. In the base conformation, the cancer-activated epitope is such a way that at high pH (e.g. pH>6.0), the epitope is occluded from binding to affinity-based reagents. Upon transition of the cell-surface biomarker to low pH in a diseased microenvironment (e.g. <6.0, such as in the acidic tumor microenvironment), the sequences proximal to the surface bound protein become glycosylated, stabilizing the framework which expresses the epitope, and simultaneously disrupts the association between the distal pH-sensitive helix with the cell surface receptor, allowing the epitope to become accessible to binding, e.g. by antibodies or binding fragments of antibodies configured to bind the epitope. Administration of antibodies or ligands that bind the epitope can be used to detect, image, or bind the newly accessible epitope and thus the assess presence of the accessible epitope in a diseased environment.

FIG. 4 (FIG. 4) depicts function of a third cell-surface biomarker containing an activatable epitope according to some embodiments described herein. In this embodiment, a natural cell-surface receptor (e.g. SSTR2) is engineered with multiple pH-sensitive helices on the terminus of the protein, which co-associate and occlude the receptor's ligand binding state (“epitope”) at high pH. Upon transition of the receptor to low pH (e.g. in a tumor microenvironment), the pH-sensitive helices dissociate, allowing binding of a detectable synthetic analog of a natural ligand (e.g. 68Ga-DOTATATE labeled somatostatin “DOTATATE”, which can be detected by PET/CT).

FIG. 5 (FIG. 5) depicts an example design for a secretable membrane-binding reporter molecule as described in Example 3.

FIG. 6 (FIG. 6) depicts a proposed mechanism of function for a secretable membrane-binding reporter molecule as described in Example 3.

FIG. 7 (FIG. 7) depicts the experiment described in Example 3 demonstrating that secretable membrane-binding reporter molecules as in FIG. 5 and FIG. 6 propagate through medium to untransfected cells.

FIG. 8A (FIG. 8A) depicts a schematic for engineered delta-like ligand 3 (DLL3) surface presentation constructs (top) and linear construct designs from N- to C-terminus of NP244, NP245, NP246, NP247, NP248, and NP249 (bottom). DLL3 constructs were engineered by supplanting domains of DLL3 protein, which serve as antigen targets, onto molecular scaffolds in which transmembrane anchoring domains serve to tether the DLL3 onto the cell surface. SP denotes the presence of a signaling peptide used to drive the expression of the engineered DLL3 protein to the cell surface. The structure of DLL3 wild-type (WT) with an SP is depicted on the far left. Two strategies were evaluated for enhanced surface trafficking: utilizing surface trafficking directives (signal peptide or transmembrane anchoring domain) from proteins known to be well expressed on the surface of lung cancer cells (B1, C1, and D1), or surface trafficking directives commonly used for surface presentation of chimeric antigen receptors on immune cells (E1, F1, and G1). E1 includes an hIgG4 Fc domain, and F1 and G1 include a CD8a hinge as extracellular scaffolds to aid in presentation of the DLL3 epitope.

FIG. 8B (FIG. 8B) depicts the amount of DLL3 protein that can be measured by FACS analysis on the cell surface after transient transfection with DNA expression plasmids that express engineered DLL3 variants. A fluorescently labeled antibody against DLL3 was used as the detection agent in this experiment. In A2, which corresponds to A1 in FIG. 8A, an H1299 cell line derived from human cancer was transfected with a formulated DNA nano-plasmid that expressed the wild-type DLL3 protein. Panels B2-G2 show the corresponding levels of DLL3 produced on the cell surface when the similar plasmids were introduced into H1299 cells. The data in FIG. 8B matches the scaffolds in which DLL3 was modeled in FIG. 8A. B2 did not increase the expression level of DLL3. C2-G2 resulted in increased expression level of DLL3 relative to the wild-type variant in B2.

FIG. 8C (FIG. 8C) shows detection of engineered DLL3 on the surface of H1299 cells. All engineered constructs except NP244 (B1 in FIG. 8A) increased DLL3-positive cells above wild-type DLL3 levels (red line representing NP116). The top two constructs, NP249 and NP247 (D1 and G1, respectively, in FIG. 8A), increased percent positive to ˜80% and ˜94%, respectively. NP247 (G1 in FIG. 8A) also displayed significantly higher mean fluorescence intensity compared to DLL3 wild-type or the DLL3 stable cell line suggesting an increased amount of DLL3 protein may be trafficked to the cell surface. H1299-DLL3: stable DLL3 expressing cell line. NP116: SURV-DLL3 WT. NP127: CAG-DLL3 WT. GMFI: Geometric Mean Fluorescence Intensity.

FIG. 8D (FIG. 8D) shows in vitro cell killing assay. H1299 cells were transfected with NP247 or NP249, and the ability of an anti-DLL3 antibody conjugated with a cytotoxic payload (PBD) to induce cytotoxicity was determined. NP247 and NP249 expressing cells underwent substantial loss of viability when treated with anti-DLL3 antibody conjugated with PBD (anti-DLL3-PBD). This suggests that cells expressing NP247 or NP249 DLL3 constructs may be more susceptible to cell death when treated with anti-DLL3-PBD compared to cells transfected with wild-type DLL3 (e.g., NP116 or NP127).

FIG. 9A (FIG. 9A) depicts additional protein changes that have been bioengineered into NP247 (G1 in FIG. 8A), or CD8 short, which served as the parental sequence for the second design set (top) and linear construct designs from N- to C-terminus of NP296, NP298, NP299, NP302, NP303, and NP304 (bottom). NP296, NP297, and NP298 were engineered to identify a ‘minimal domain’ of DLL3 sufficient for interaction with the antibody tracer. Importantly, NP298 was engineered to include a second epitope onto the structure in an attempt to double the antibody binding capacity. NP299 was engineered to comprise two minimal epitopes of DLL3 inserted the extracellular domain of DLL3. NP302, NP303, NP304 were engineered by swapping the C2 domain of DLL3 with a more soluble humanized nanobody structure. NP303 and NP304 additionally comprise two minimal epitopes of DLL3 in the soluble humanized nanobody structure; and NP304 does not comprise and extracellular DLL3 domain.

FIG. 9B (FIG. 9B) depicts the levels of DLL3 expression on the cell surface following transfection into H1299 cells. As in FIG. 8B, these data demonstrate that DLL3 modeled into a molecular scaffold produces higher expression levels of surface DLL3 than H1299 transfected with a plasmid with the wild-type DLL3 protein. A fluorescently labeled antibody against DLL3 was used as the detection agent in this experiment. The refinement of the structure to include the presence of a humanized soluble nanobody domain in NP302 further enhances expression over NP247. Importantly, the addition of two DLL3 binding domains on NP298 indicates that antibody binding capacity can be increased by a multivalent approach and boost overall levels of antibody binding activity.

FIG. 9C (FIG. 9C) depicts the levels of DLL3 expression from NP247, NP302, NP304, NP296, and NP298 constructs on the cell surface following transfection into H1299 cells. The graph on the left shows percentage of DLL3-positive cells and the graph on the right shows geometric mean fluorescent intensity (GMFI) of DLL3-positive cells. A fluorescently labeled antibody against DLL3 was used as the detection agent in this experiment.

FIGS. 10A and 10B (FIG. 10A and FIG. 10B) depict the amount of DLL3 that can be measured by FACS analysis on the cell surface following transfection of H1299 cells with the wild-type DLL3 epitope, NP247, which expresses a single epitope in the CD8 scaffold, or NP298, which expresses two epitopes of DLL3 in a CD8 scaffold. A fluorescently labeled antibody against DLL3, Phycoerythrin (PE, FIG. 10A) or Fluorescein isothiocyanate (FITC, FIG. 10B), was used as the detection agent in this experiment. Approximate antibody binding per cell was assessed using a standard curve (FIG. 10B).

FIG. 10C (FIG. 10C) depicts the physical number of antibody copies bound to the DLL3 expressed on the cell surface following transfection of H1299 cells with the wild-type DLL3 epitope, NP247, which expresses a single epitope in the CD8 scaffold, or NP298, which expresses two epitopes of DLL3 in a CD8 scaffold. A standard curve was used to estimate antibody binding. NP298 showed a 32-fold increase over the levels of the wild-type DLL3 epitope expression. A fluorescently labeled antibody against DLL3 was used as the detection agent in this experiment.

FIG. 10D (FIG. 10D) depicts geometric mean intensity of internalized DLL3 following transfection of H1299 cells with NP116 (wild-type DLL3) or NP247. A fluorescently labeled antibody against DLL3 was used as the detection agent in this experiment.

FIG. 11 (FIG. 11) shows an image taken from a positron emission tomography (PET) scan. In this model system, a DNA plasmid that expresses a copy of the somatostatin receptor 2 (SSTR2) gene was generated and then stably introduced into H1299 cells such that each of the cell lines contained 2 genomic copies of the stably integrated SSTR2 construct. With the goal of ascertaining the minimal number of cells to produce a measurable signal, mice were implanted subcutaneously with different numbers of engineered cells with stably integrated SSTR2 at separate locations and then treated with a 68Ga-dotate positron-emitting tracer that binds to SSTR2 protein on the cell surface. These data illustrate that as little as 31,000 cancer cells can be detected in this mouse model when cells are induced to express the surrogate biomarker on the cell surface.

FIG. 12 (FIG. 12) shows an image taken from a single-photon emission computed tomography (SPECT) scan. In this model system, a DNA plasmid that expresses a copy of SSTR2 gene was generated and then stably introduced into H1299 cells such that each of the cell lines contained 2 genomic copies of the stably integrated SSTR2 construct. Mice were implanted subcutaneously with engineered cells with stably integrated SSTR2 and then treated with a ¹⁷⁷LU-dotate beta-emitting tracer that binds to SSTR2 protein on the cell surface. These data illustrate that high energy tracers can be used for detecting biomarkers on cells that are engineered to express those biomarkers. Middle: coronal image sectioned at kidneys and tumor. Right: maximum intensity projection (MIP). 4H, 24H, and 120H: 4 hours, 24 hours, and 120 hours post tracer dosing, respectively.

FIG. 13A (FIG. 13A) depicts the physical number of antibody copies bound to the DLL3 expressed on the cell surface (left) and geometric mean intensity of internalized DLL3 following transfection of H1299 cells with NP116 (wild-type DLL3), NP247, or NP298. A standard curve was used to estimate antibody binding. NP247 showed a 21-fold increase over the levels of the wild-type DLL3 epitope expression. NP298 showed a 37-fold increase over the levels of the wild-type DLL3 epitope expression. A fluorescently labeled antibody against DLL3 was used as the detection agent in this experiment.

FIG. 13B (FIG. 13B) depicts in vitro cell killing assay. H1299 cells were transfected with NP116, NP247, or NP298, and the ability of an anti-DLL3 antibody conjugated with a cytotoxic payload (PBD) to induce cytotoxicity was determined. NP247 and NP298 expressing H1299 cells underwent substantial loss of viability when treated with anti-DLL3 antibody conjugated with PBD (anti-DLL3-PBD). This suggests that cells expressing NP247 or NP298 DLL3 constructs may be more susceptible to cell death when treated with anti-DLL3-PBD compared to cells transfected with wild-type DLL3 (NP116).

FIG. 14 shows an image taken from a single-photon emission computed tomography (SPECT) scan. In this model system, a DNA plasmid that expresses a copy of SSTR2 gene was generated and then stably introduced into H1299 cells using a lentiviral system. Mice were implanted subcutaneously with engineered cells with stably integrated SSTR2 construct, treated with a 200 μCi [⁶⁸GA]-DOTATATE tracer that binds to SSTR2 protein on the cell surface, and PET/CT imaged 2 hours post tracer administration.

FIG. 15 (FIG. 15) depicts a schematic of an exemplary workflow of the methods described herein. A DNA (1) construct that uses activated expression of a cell surface protein can be introduced into the target cell population by transient transfection (2). After being transported in the nucleus and unpacking, the DNA acts as a nucleic acid template for transcription (3-5) and produces an engineered reporter gene. Further protein modifications can occur in the cytoplasm (7) or on the cell surface (9) in response to the molecular disease status of the cell. Alternatively, the protein can be secreted in the extracellular milieu for propagation of the signal into neighboring cells (8). Once on the cell surface, the target antigen can be easily detected with affinity-based reagents that enable detection by imaging techniques involving radiotracers or other modalities. Using high energy emitters also offers the possibility of therapeutic applications.

### DETAILED DESCRIPTION

### Overview

In some aspects, the present disclosure concerns encodable (e.g. under the control of a tumor- or cancer-specific promoter) cell-surface synthetic biomarkers that contribute to the identification, localization, or treatment of tumor cells in a subject.

In some cases, such encodable cell-surface synthetic biomarkers comprise: (a) an extracellular domain configured to bind an affinity reagent, and (b) a membrane binding or transmembrane domain. When provided encoded on a nucleic acid under the control of a cancer-specific promoter (e.g. a promoter of a gene overexpressed in a cancer cell relative to a normal cell), administration of the nucleic acid to a subject suspected of having cancer drives production of the cell-surface biomarker selectively in cancerous cells. The extracellular domain can comprise a terminal activatable epitope or a ligand binding domain. Administration of a suitable affinity reagent (e.g. a ligand binding the ligand binding domain or an affinity reagent/antibody that binds the epitope) to the subject can then be used to treat the tumorous cells selectively express the encoded cell-surface synthetic biomarker (if the affinity reagent is a suitable therapeutic antibody) or localize or identify the cells (if the affinity reagent is e.g. a labeled antibody).

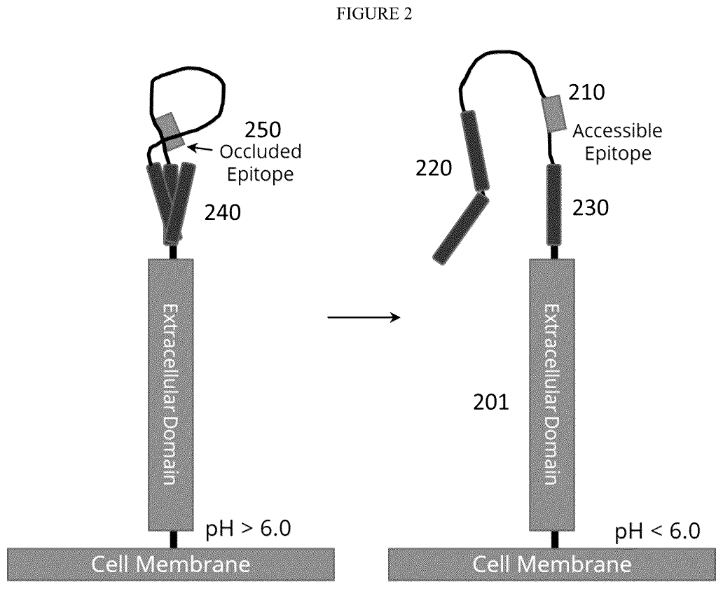

One example configuration for such a cell-surface synthetic biomarker is provided in FIG. 2, where binding of an affinity reagent is dependent on cancer-associated transcriptional and extracellular conditions. In this configuration, the cell-surface synthetic biomarker comprises an extracellular domain (201 e.g. derived from any of the receptors described herein) attached to a membrane via a transmembrane domain; the extracellular domain having a terminal epitope (210) flanked by two compatible portions of a pH-sensitive multimerization domain (220 and 230), which can be pH-sensitive helices as described herein. Proper external localization of the extracellular domain can be driven by providing a signal peptide at the terminus of the extracellular domain. At high pH, self-association of the pH-sensitive multimerization domain (240) occludes the epitope (250) from binding to an affinity reagent (e.g. an affinity reagent administered to a subject). At low pH, dissociation of the two compatible portions of a pH-sensitive multimerization domain (220 and 230) allows the epitope (210) to be accessible to an extracellular affinity reagent. In this manner, the cell-surface synthetic biomarker can be dependent on both the transcriptional status of the cancer cell (e.g. via expression from a cancer-specific promoter, or a promoter of a gene overexpressed in a cancer cell relative to a normal cell) and the typical acidic extracellular environment of tumors. The combination of transcriptional and extracellular conditions for binding to the affinity reagent can result in improved specificity of targeting the cancer in the subject.

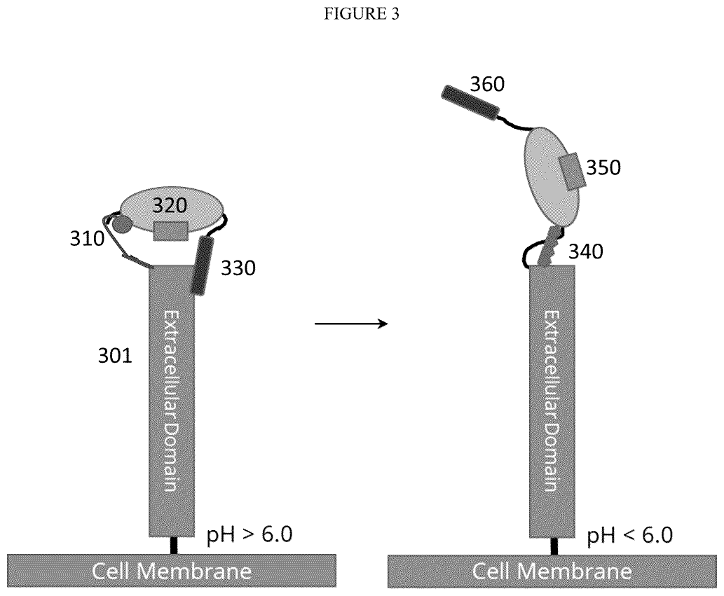

Another example configuration for such a cell-surface synthetic biomarker is provided in FIG. 3. In this case the configuration is similar to that of FIG. 2, except that the extracellular domain (301) is configured to bind to a pH-sensitive helix (330) at low pH, and the activatable epitope (320) is flanked by the pH-sensitive helix (330) and a cancer-associated post-translational modification site (310), the cancer-associated post-translational modification site contributing to stabilization of the activatable epitope when post-translationally modified. In this manner, accessibility of the activatable epitope can be controlled in a manner dependent on both extracellular pH via the pH-sensitive helix, and cancer-associated extracellular enzyme expression, if the post-translational modification site is chosen as one selectively targeted by extracellular enzymes expressed in a cancer. In this design, in the absence of the post-translational modification and low pH (e.g. in a non-cancer microenvironment), the activatable epitope (320) is excluded. In contrast, in a cancer-associated extracellular milieu, the activatable epitope (350) is accessible via dissociation of the pH-sensitive helix (360) and post-translational modification of the flanking post-translational modification site (340).

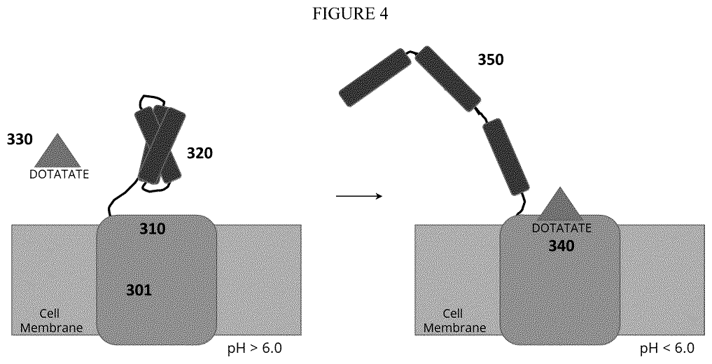

Another example configuration for such a cell-surface synthetic biomarker is provided in FIG. 4, wherein configuration of the biomarker incorporates a ligand binding domain to allow the use of a modified natural ligand for the ligand binding domain to be used as an affinity reagent. In this configuration, a membrane bound ligand-binding domain (301) is provided linked to a terminal pH-sensitive helix (320) that occludes binding of a ligand (330) to the ligand binding site (310) at low pH. At high pH (e.g. non-cancer microenvironments), the pH-sensitive helix (350) dissociates and allows binding of the ligand to the ligand binding site (340) on the surface of the cell. The combination of transcriptional (when the biomarker is encoded under control of a cancer-specific promoter or a promoter of a gene overexpressed in a cancer cell relative to a normal cell), extracellular pH, and extracellular post-translational conditions required to activate the epitope for binding in this example can result in improved specificity of targeting the cancer in the subject.

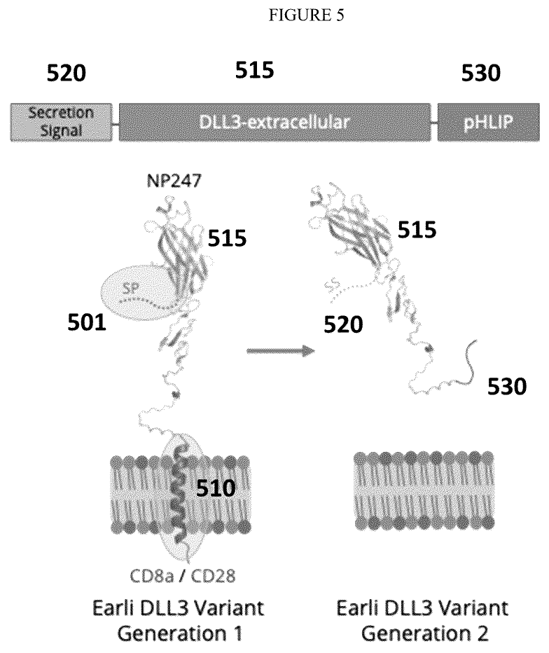

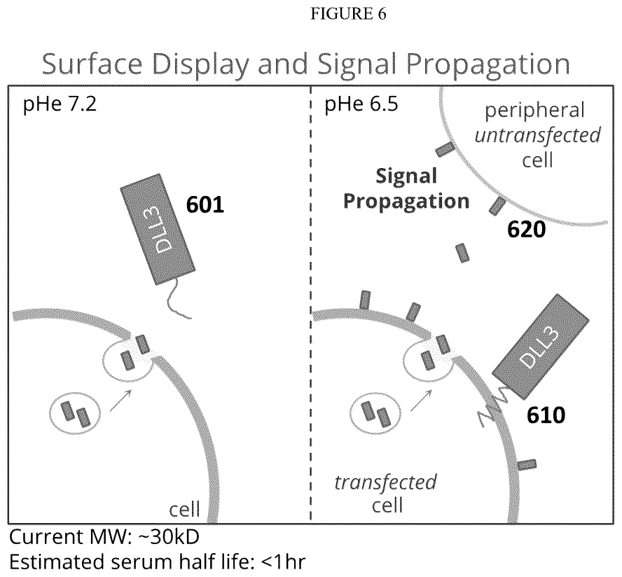

Another example configuration for such a cell-surface synthetic biomarker is provided in FIG. 5, wherein a receptor of the type described in FIG. 2 (501, 515, 510) is modified to replace a signal peptide (501) with a secretion signal (520), and a transmembrane domain (510) with a membrane binding domain (530) e.g. a pH-sensitive membrane-binding helix) to yield a second generation cell-surface synthetic biomarker (520, 515, 530) capable of propagation to neighboring cells when expressed by a cancer cell (e.g. under a cancer-cell specific promoter). Such a cell-surface synthetic biomarker is thus configured to function as shown in FIG. 6, wherein secreted biomarkers are secreted in the extracellular fluid (601) and are unattached to cells if secreted in a non-acidic extracellular environment (e.g. a non-tumor environment) but attach to both the secreting cell (610) and neighboring cells (620) when at the proper pH to allow for assembly of the membrane binding helix. The combination of dependence of extracellular conditions to label cells and the ability to propagate to neighboring cells can result in improved specificity and sensitivity of targeting the cancer for this configuration.

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

The term “or”, as used herein, is generally intended as an inclusive for, encompassing both alternatives as well as either.

Included in the current disclosure are variants of any of the polypeptides described herein with one or more conservative amino acid substitutions. Such conservative substitutions can be made in the amino acid sequence of a polypeptide without disrupting the three-dimensional structure or function of the polypeptide, or with only minimal disruption to function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the polypeptide protein sequences described herein. In some embodiments, such conservatively substituted variants are functional variants. Such functional variants can encompass sequences with substitutions such that the activity of one or more critical active site residues or structural supporting residues of the polypeptide are not disrupted.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1,000,000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “subject” can generally include human or non-human animals. Thus, the methods and compositions described herein are applicable to both human and veterinary disease and animal models. Preferred subjects are “patients,” i.e., living humans that are receiving medical care for a disease or condition. This includes persons with no defined illness who are being investigated for signs of pathology. Also included are persons suspected of possessing or being at-risk for a defined illness. In some embodiments, the subject has at least one risk factor for cancer such as Li-Fraumeni syndrome, lynch syndrome, familial adenomatous polyposis, lung nodules, Von Hippel-Lindau disease, aplastic anemia, myelodysplastic syndrome, Cowden syndrome, hereditary breast and ovarian cancer syndrome (HBOC), or BRCA mutations; being a current smoker, ex-smoker, or exposed to heavy doses of second hand smoke; exposure to carcinogens, excessive sunlight, immunosuppressive agents, or hepatitis B, hepatitis C, or human papilloma virus; or obesity.

A “vector” as used herein generally refers to a nucleic acid sequence capable of transferring other operably-linked heterologous or recombinant nucleic acid sequences to target cells. In some examples, a vector is a minicircle, plasmid, nanoplasmid, yeast artificial chromosome (YAC), bacterial artificial chromosome (BAC), cosmid, phagemid, bacteriophage genome, or baculovirus genome. Suitable vectors also include vectors derived from bacteriophages or plant, invertebrate, or animal (including human) viruses such as CELiD vectors, doggybone DNA (dbDNA) vectors, closed-end linear duplex DNA vectors (e.g., wherein each end is covalently closed by chemical modification), adeno-associated viral vectors (e.g. AAV1, AAV2, AAV4, AAV5, AAV6, AAV7, AAV8, AAV9, or pseudotyped combinations thereof such as AAV2/5, AAV2/2, AAV-DJ, or AAV-DJ8), retroviral vectors (e.g. MLV or self-inactivating or SIN versions thereof, or pseudotyped versions thereof), herpesviral (e.g. HSV- or EBV-based), lentiviral vectors (e.g. HIV-, FIV-, or EIAV-based, or pseudotyped versions thereof), or adenoviral vectors (e.g. Ad5-based, including replication-deficient, replication-competent, or helper-dependent versions thereof). In some embodiments, a vector is a replication competent viral-derived vector. In some embodiments, a vector is a replication-incompetent viral-derived vector. In some cases, the vector may comprise an episomal maintenance element to facilitate replication in one or more target cell type, such as a Scaffold/Matrix Attachment Region (S/MAR). S/MAR elements are particularly useful to facilitate replication in the context of “naked” nucleic acid vectors such as minicircles. Exemplary suitable S/MAR elements include, but are not limited to, EμMAR from the immunoglobulin heavy chain locus, the apoB MAR from the human apolipoprotein B locus, the Ch-LysMAR from the chicken lysozyme locus, and the huIFNβ MAR from the human IFNβ-locus. A vector may comprise a coding sequence capable of being expressed in a target cell. Accordingly, as used herein, the terms “vector construct,” “expression vector,” and “gene transfer vector,” may refer to any nucleic acid construct capable of directing the expression of a gene of interest and which is useful in transferring the gene of interest into target cells. Vectors as described herein may additionally comprise one or more cis-acting elements to stabilize or improve expression of mRNAs therefrom. Such cis-acting elements include but are not limited to any of the elements described e.g., in Johansen et al. The Journal of Gene Medicine. (5) 12:1080-1089 (doi: 10.1002/jgm.444) or Vlasova-St. Louis and Sagarsky. Mammalian Cis-Acting RNA Sequence Elements (doi: 10.5772/intechopen.72124).

As one of the forms of vectors, the term “minicircle” as used herein generally refers to a small, double stranded circular DNA molecule that provides for persistent, high level expression of a sequence of interest that is present on the vector, which sequence of interest may encode a polypeptide, an shRNA, an anti-sense RNA, an siRNA, and the like. The sequence of interest is operably linked to regulatory sequences present on the minicircle vector, said regulatory sequences controlling its expression. Such minicircle vectors are described, for example in published U.S. Patent Application US20040214329, herein specifically incorporated by reference. As a different form of vectors, “nanoplasmid” refers to a vector that may comprise minimized bacterial ColE1 or R6K origin of replication (which provides for such nanoplasmids to be replicable in a bacterial host strain), a bacterial RNA-based selectable marker, and a eukaryotic gene region. Further examples of such elements (nanoplasmid origins and RNA-out selectable markers) are described e.g., in U.S. Pat. No. 9,737,620B2, which is incorporated by reference herein for the purposes of describing nanoplasmid sequence elements.

The overall length of a minicircle vector is sufficient to include the desired elements as described below, but not so long as to prevent or substantially inhibit to an unacceptable level the ability of the vector to enter a target cell upon contact with the cell, e.g., via system administration to the host comprising the cell. As such, the minicircle vector can be generally at least about 0.3 kb long, often at least about 1.0 kb long, whereas the parental vector may be as long as 6 kb, 10 kb, or longer.

Minicircle vectors differ from bacterial plasmid vectors in that they lack an origin of replication or lack a natural origin of replication (e.g. may comprise a minimized synthetic bacterial origin of replication), and lack a selection marker commonly found in bacterial plasmids, e.g. p-lactamase, tetracycline-resistance (tet), kanamycin-resistance (kan), or other antibiotic selection markers. Consequently, a minicircle becomes smaller in size, allowing more efficient delivery. Minicircles lack the transgene expression silencing effect which is associated with the vector backbone nucleic acid sequences of parental plasmids from which the minicircle vectors are excised. The minicircle may be substantially free of vector sequences other than the recombinase hybrid product sequence, and the sequence of interest, i.e. a transcribed sequence and regulatory sequences required for expression.

The term “nanoplasmid” as used herein generally refers to a vector that can comprise minimized bacterial ColE1 or R6K origin of replication (which provides for such nanoplasmids to be replicable in a bacterial host strain), a bacterial RNA-based selectable marker, and a eukaryotic gene region. Some embodiments of nanoplasmids are described in e.g. US20150275221A1. In some embodiments, the nanoplasmid may comprise a fusion bacterial-RNA-based selectable marker/minimized origin of replication. In some embodiments, the fusion bacterial-RNA-based selectable marker/minimized origin of replication may be located within a synthetic intron located within the eukaryotic gene region of the nanoplasmid.

An RNA-based selectable marker is generally a vector-borne expressed non translated RNA that regulates a chromosomally expressed target gene to afford selection of the vector. This may be a plasmid borne nonsense suppressing tRNA that regulates a nonsense suppressible selectable chromosomal target as described by Crouzet J and Soubrier F 2005 U.S. Pat. No. 6,977,174 included herein by reference. This may also be a plasmid borne antisense repressor RNA, an RNA-OUTgene that represses RNA-IN regulated targets, pMBI plasmid origin encoded RNAI that represses RNAII regulated targets, IncB plasmid pMU720 origin encoded RNAI that represses RNA II regulated targets, ParB locus Sok of plasmid RI that represses Hok regulated targets, Flm locus FlmB of F plasmid that represses flmA regulated targets, another natural antisense repressor RNA such as those described in e.g. Wagner E G H, Altuvia S, Romby P. 2002. Adv Genet 46:361 and Franch T, and Gerdes K. 2000. Current Opin Microbiol 3:159, or an engineered repressor RNA such as a small synthetic small RNA like the SgrS, MicC or MicF scaffolds as described in Park et al. Nature Biotechnology volume 31, pages 170-174 (2013).

The term “transfection agent” generally encompasses any compound that mediates incorporation of DNA or RNA into a host cell, e.g., a liposome. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), Ausubel, et al., Short Protocols in Molecular Biology, 3rd ed., Wiley & Sons, 1995, and other laboratory manuals, which are hereby incorporated by reference. Examples of suitable transfection agents include, but are not limited to, linear or branched polyethylenimines, nanoparticles, liposomes, lipophilic particles, solid nanoparticles, amphipathic peptides, micelles, dendrimers, polymeric compositions, hydrogels, synthetic or naturally derived exosomes, virus-like particles, or any combination thereof.

The terms “nucleic acid molecule” and “polynucleotide” as used herein generally refer to polymeric forms of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The term “promoter” generally refers to a DNA sequence that directs the transcription of a polynucleotide. Typically, a promoter can be located in the 5′ region of a polynucleotide to be transcribed, proximal to the transcriptional start site of such polynucleotide. More typically, promoters are defined as the region upstream of the first exon; more typically, as a region upstream of the first of multiple transcription start sites. Frequently promoters are capable of directing transcription of genes located on each of the complementary DNA strands that are 3′ to the promoter. Stated differently, many promoters exhibit bidirectionality and can direct transcription of a downstream gene when present in either orientation (i.e. 5′ to 3′ or 3′ to 5′ relative to the coding region of the gene). Additionally, the promoter may also include at least one control element such as an upstream element. Such elements include upstream activator regions (UARs) and optionally, other DNA sequences that affect transcription of a polynucleotide such as a synthetic upstream element. Some promoters may be assembled from fragments of endogenous promoters (e.g. derived from the human genome).

The term “coding sequence” and “encodes” when used in reference to a polypeptide herein generally refer to a nucleic acid molecule that is transcribed (in the case of DNA) and translated (in the case of mRNA) into a polypeptide, for example, when the nucleic acid is present in a living cell (in vivo) and placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral, eukaryotic, or prokaryotic DNA, and synthetic DNA sequences. A transcription termination sequence may be located 3′ to the coding sequence, and a promoter may be located 5′ to the coding sequence; along with additional control sequences if desired, such as enhancers, introns, poly adenylation site, etc. A DNA sequence encoding a polypeptide may be optimized for expression in a selected cell by using the codons preferred by the selected cell to represent the DNA copy of the desired polypeptide coding sequence.

The term “operably linked” as used herein generally refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The term “pharmaceutically acceptable carrier” as used herein generally refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “antibody” or “immunoglobulin” generally includes five distinct classes of antibody that can be distinguished biochemically. With regard to the IgG class of immunoglobulin molecules, which can be considered prototypical, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable domain.

Light chains of an immunoglobulin can be classified as either kappa or lambda (κ, λ). Each heavy chain class can be bound with either a kappa or lambda light chain. In general, the light and heavy chains can be covalently bonded to each other, and the “tail” portions of the two heavy chains can be bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Heavy chains can be classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. can confer functional specialization.

Both the light and heavy chains can be divided into regions of structural and functional homology. The term “region” when used to describe an immunoglobulin or related molecule generally refers to a part or portion of a single immunoglobulin (as is the case with the term “Fc region”) or a single antibody chain and includes constant regions or variable regions, as well as more discrete parts or portions of said domains. For example, light chain variable domains include “complementarity determining regions” or “CDRs” interspersed among “framework regions” or “FRs”, as defined herein.

Certain regions of an immunoglobulin can be defined as “constant” (C) regions or “variable” (V) regions, based on the relative lack of sequence variation within the regions of various class members in the case of a “constant region”, or the significant variation within the regions of various class members in the case of a “variable regions”. The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like.

The constant and variable regions of immunoglobulin heavy and light chains can be folded into domains. The term “domain” generally refers to an independently folding, globular region of a heavy or light chain polypeptide comprising peptide loops (e.g., comprising 3 to 4 peptide loops) stabilized, for example, by β-pleated sheet and/or intrachain disulfide bond. Constant region domains on the light chain of an immunoglobulin can be referred to interchangeably as “light chain constant region domains”, “CL regions” or “CL domains”. Constant domains on the heavy chain (e.g. hinge, CH1, CH2 or CH3 domains) can be referred to interchangeably as “heavy chain constant region domains”, “CH” region domains or “CH domains”. Variable domains on the light chain can be referred to interchangeably as “light chain variable region domains”, “VL region domains or “VL domains”. Variable domains on the heavy chain are referred to interchangeably as “heavy chain variable region domains”, “VH region domains” or “VH domains”.

By convention the numbering of the variable and constant region domains increases as they become more distal from the antigen binding moiety or amino-terminus of the immunoglobulin or antibody. The N-terminus of each heavy and light immunoglobulin chain is a variable region and at the C-terminus is a constant region; the CH3 and CL domains comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

As used herein, the terms “CH2 domain sequence”, “CH3 domain sequence”, “CH1 domain sequence”, and “CL domain sequence” generally refer to polypeptide sequences that are derived from the CH2 domain, CH3 domain, CH1 domain and CL domain, respectively, of a native immunoglobulin molecule.

Amino acid positions in a heavy chain constant region, including amino acid positions in the CL, CH1, hinge, CH2, and CH3 domains, can be numbered herein according to the EU index numbering system (see e.g. Kabat et al., in “Sequences of Proteins of Immunological Interest”, U.S. Dept. Health and Human Services, 5th edition, 1991, which is incorporated by reference herein).

As used herein, the term “VH domain” generally includes the amino terminal variable domain of an immunoglobulin heavy chain, and the term “VL domain” includes the amino terminal variable domain of an immunoglobulin light chain.

As used herein, the term “CH1 domain” generally includes the first (most amino terminal) constant region domain of an immunoglobulin heavy chain that extends, e.g., from about EU positions 118-215. The CH1 domain is adjacent to the VH domain and amino terminal to the hinge region of an immunoglobulin heavy chain molecule, and is not considered part of the Fc region of an immunoglobulin heavy chain.

As used herein, the term “hinge region” or “hinge domain” generally includes the portion of a heavy chain molecule that joins the CH1 domain to the CH2 domain, or a domain of a non-heavy chain molecule with sequence similarity to the portion of a heavy chain molecule that joins the CH1 domain to the CH2 domain. This hinge region is flexible, thus allowing the two N-terminal antigen binding regions to move independently. Hinge regions can be subdivided into three distinct domains: upper, middle, and lower hinge domains (see e.g. Roux et al. J. Immunol. 1998, 161:4083, which is incorporated by reference herein). The upper hinge domain generally refers to the portion of a heavy chain molecule stretching from the C-terminal end of the CH1 domain to the first hinge cysteine. The middle hinge domain generally refers to the portion of a heavy chain molecule stretching from the first to the last cysteine in the hinge. The lower hinge domain generally refers to the portion of a heavy chain molecule stretch from the last cysteine in the hinge to Gly-237 according to EU numbering in the CH2 domain.

As used herein, the term “hinge region” or “hinge domain” generally includes the portion of a CD8 molecule (e.g. a CD8a or CD8b molecule) following beta-pleated sheet immunoglobulin V-domain like regions which is rich in proline, threonine and serine residues and has significant sequence identity to immunoglobulin heavy chain hinge regions or hinge domains.

As used herein, the term “CH2 domain” generally includes the portion of a heavy chain immunoglobulin molecule that extends, e.g., from about EU positions 231-340. The CH2 domain is not closely paired with another domain. Rather, two N-linked branched carbohydrate chains are interposed between the two CH2 domains of an intact native IgG molecule.

As used herein, the term “CH3 domain” generally includes the portion of a heavy chain immunoglobulin molecule that extends approximately 110 residues from C-terminus of the CH2 domain, e.g., from about position 341-447 (EU numbering system). The CH3 domain typically forms the C-terminal portion of the antibody. In some immunoglobulins, however, additional domains may extend from CH3 domain to form the C-terminal portion of the molecule (e.g. the CH4 domain in the u chain of IgM and the c chain of IgE).

As used herein, the term “CL domain” generally includes the first (most amino terminal) constant region domain of an immunoglobulin light chain that extends, e.g. from about EU position 108-214. The CL domain is adjacent to the VL domain.

As used herein, the term “Fc domain” generally refers to the portion of a single immunoglobulin heavy chain beginning in the hinge region just upstream of the papain cleavage site (i.e. residue 216 in IgG, taking the first residue of heavy chain constant region to be 118) and ending at the C-terminus of the antibody. Accordingly, a complete Fc domain comprises at least a hinge domain, a CH2 domain, and a CH3 domain.

As used herein, the term “antibody or antigen-binding fragment(s) thereof” is generally used in the broadest sense and encompasses intact monoclonal antibodies, polyclonal antibodies, multispecific antibodies (e.g. bispecific antibodies) formed from at least two intact antibodies, and antibody fragments. Antibody fragments comprise a portion of an intact antibody that retains antigen-binding activity; examples include Fab, Fab′, F(ab)₂, F(abc)₂, and Fv fragments as well as diabodies, linear antibodies, scFvs, and multispecific antibodies formed from antibody fragments.

A single-chain Fv (“sFv” or “scFv”) polypeptide generally refers to a covalently linked VH-VL heterodimer which is expressed from a gene fusion including VH- and VL-encoding genes linked by a peptide-encoding linker (see e.g. Huston et al., Proc. Nat. Acad. Sci. USA (1988) 85:5879-5883). A number of methods have been described to discern and develop chemical structures (linkers) for converting the naturally aggregated, but chemically separated, light and heavy polypeptide chains from an antibody V region into an sFv molecule which will fold into a three-dimensional structure substantially similar to the structure of an antigen-binding site. See, e.g., U.S. Pat. Nos. 5,091,513, 5,132,405 and 4,946,778.

As used herein, the term “epitope” generally refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of many documented methods (e.g., Giege R. et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Antibody: antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies may be accomplished using any of several documented methods, such as those described in e.g., Champe M et al., (1995) J Biol Chem 270:1388-1394 and Cunningham B C & Wells J A (1989) Science 244:1081-1085

As used herein, the term “linker domain” generally refers to a sequence which connects two or more domains in a linear sequence. In some embodiments, any of the domains or regions described herein can be connected to each other by a linker domain, which can comprise e.g. (GGS)n or (GGGS)n (SEQ ID NO: 92).

The term “transmembrane domain” or “transmembrane region” generally includes an amino acid sequence of about 15 amino acid residues in length which spans the plasma membrane. A transmembrane domain or region can also include about at least 20, 25, 30, 35, 40, or 45 amino acid residues and span the plasma membrane. Transmembrane domains can be rich in hydrophobic residues, and have an alpha-helical structure. In one embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al. (1996) Annual Rev. Neurosci. 19:235-263, the contents of which are incorporated herein by reference.

### Epitopes

In some aspects, provided herein is an engineered polypeptide comprising an extracellularly-oriented domain (e.g., extracellular domain) comprising an epitope. An extracellular domain can optionally comprise a signal peptide. In some embodiments, an engineered polypeptide sequence can comprise a sequence for an extracellular domain sequence containing an epitope and a sequence for an optional signal peptide sequence encoded by a single nucleic acid sequence. In some embodiments, a signal peptide sequence can be cleaved after expression of the engineered polypeptide. In some embodiments, an engineered polypeptide sequence can be designed to comprise a signal peptide that is heterologous to the extracellular domain of the engineered polypeptide.

In some embodiments, an epitope can bind to an antibody. In some embodiments, an epitope can bind to a peptide hormone or growth factor. In some embodiments, an epitope can be derived from a biomarker. In some embodiments a biomarker can be a biomarker for a disease or a condition, for example, a cancer. In some embodiments, an epitope can be derived from a biomarker for a cancer comprising Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, Uveal Melanoma, lip melanoma, spindle cell carcinoma, liposarcoma, nasal sarcoma, mammary adenocarcinoma, insulinoma, osteosarcoma, mast cell tumors, hemangiosarcoma, non-small cell lung carcinoma (NSCLC), marginal lymphoma, malignant melanoma, or chronic lymphocytic leukemia. In some embodiments, an epitope can be derived from DLL3, PSMA, SSTR2, CD8a, CD4, TGFR1, IGF1R, PD-L1, EGFR, CD73, IgK, IL-6, or any combination thereof. In some embodiments, an epitope can be derived from DLL3, PSMA, SSTR2, or any combination thereof.

Other examples of proteins from which an epitope can be derived can include, but are not limited to, Exotoxin A, Epstein-Barr nuclear antigen 4, Melanoma antigen recognized by T-cells 1, 65 kDa phosphoprotein, Immediate early protein IE1, Trans-activator protein BZLF1, Prostate-specific antigen, Cellular tumor antigen p53 (UniProt: A0A0U1RQC9), Envelope glycoprotein B, Melanoma-associated antigen 1, Tyrosinase, Epstein-Barr nuclear antigen 1, Protein K8.1, Spike glycoprotein, Latent membrane protein 1, Protein Tax-1, Epstein-Barr nuclear antigen 3, Apoptosis regulator BHRF1, Histone H4, Melanoma-associated antigen 4, Receptor tyrosine-protein kinase erbB-2 (UniProt: P04626), Melanocyte protein PMEL, Prostatic acid phosphatase, Gag-Pro-Pol polyprotein, Keratin, type I cytoskeletal 18, L-dopachrome tautomerase, Envelope glycoprotein gp62, Thyroglobulin, Melanoma-associated antigen 3, Protein E6, POTE ankyrin domain family member F, Envelope glycoprotein B, Protein LANA1, GTP-binding protein GEM, Envelope glycoprotein gp63, Epstein-Barr nuclear antigen 6, Latent membrane protein 2, Small capsomere-interacting protein, Thymosin beta-10, Carcinoembryonic antigen-related cell adhesion molecule 5 (UniProt: P06731), Myelin basic protein (UniProt: J3QL64), Melanoma-associated antigen 2, Cytoplasmic dynein 1 heavy chain 1 (UniProt: Q14204), Tyrosine-protein kinase ABL1, Capsid protein, Wilms tumor protein (UniProt: A0A0A0MT54), Complement factor H (UniProt: P08603), G2/mitotic-specific cyclin-B1 (Fragment) (UniProt: E9PC90), Amyloid-beta precursor protein, mRNA export factor ICP27 homolog, B-lymphocyte antigen CD20 (UniProt: P11836), Protein virilizer homolog, Protein E7, Immunoglobulin heavy constant gamma 1 (Fragment) (UniProt: A0A0A0MS08), Immunoglobulin heavy constant gamma 1, Immunoglobulin heavy constant gamma 4, Endoplasmic reticulum chaperone BiP, Transferrin receptor protein 1 (UniProt: P02786), Cellular tumor antigen p53 (UniProt: P04637), Neurotrophin receptor-interacting factor homolog, U1 small nuclear ribonucleoprotein 70 kDa, Melanoma-associated antigen 6, Apolipoprotein B-100, Mammaglobin-A (UniProt: Q13296), DNA polymerase processivity factor, Gag polyprotein, TCR gamma alternate reading frame protein, 60S ribosomal protein L28 (UniProt: P46779), Glutamate carboxypeptidase 2, Cellular tumor antigen p53 (UniProt: E7EQX7), DNA (cytosine-5)-methyltransferase 1, Catenin beta-1 (UniProt: P35222), Small nuclear ribonucleoprotein-associated proteins B and B′, 60S ribosomal protein L8 (UniProt: P62917), Melanoma-associated antigen B2, Protein SSX2, Small nuclear ribonucleoprotein Sm D1, Major capsid protein L1, Tenascin (UniProt: P24821), Elongation factor 2, Matrix protein, ADP/ATP translocase 2, Stearoyl-CoA desaturase, Glutamine—fructose-6-phosphate transaminase (isomerizing), Protein Rex, Spectrin alpha chain, non-erythrocytic 1 (UniProt: Q13813), HLA class II histocompatibility antigen DR beta chain, Hom s 1, Receptor tyrosine-protein kinase erbB-2 (UniProt: B4DTR1), CCR4-NOT transcription complex subunit 9, Protein E6, Dihydrolipoyllysine-residue acetyltransferase component of pyruvate dehydrogenase complex, mitochondrial, Heat shock 70 kDa protein 1A (UniProt: P0DMV8), Annexin A1, Apoptosis regulator Bcl-2 (UniProt: P10415), Melanoma-associated antigen 9, Melanoma-associated antigen 6 (Fragment), 60S ribosomal protein L7a (UniProt: P62424), ELAV-like protein 4, Titin (UniProt: Q8WZ42), Cancer/testis antigen 1, Cellular tumor antigen p53 (UniProt: J3KP33), Mucin-1 (UniProt: A0A0C4DGW3), Keratin, type I cytoskeletal 10, Ribonucleoside-diphosphate reductase large subunit, G-protein coupled receptor 143, Bifunctional purine biosynthesis protein ATIC, Myosin-14, Fibronectin, Transcription factor HIVEP2, Histone H3 (Fragment), Histone H3.3 (UniProt: P84243), Solute carrier family 45 member 3, E3 ubiquitin protein ligase, Polypyrimidine tract-binding protein 1 (UniProt: P26599), Replication protein E1, DNA topoisomerase 2-alpha, Proteasomal ubiquitin receptor ADRM1 (UniProt: Q16186), Epidermal growth factor receptor, Golgi apparatus protein 1 (UniProt: Q92896), Elongation factor 1-alpha 1, Elongation factor 1-alpha 2, Tyrosine-protein kinase JAK1, DEK (UniProt: P35659) & Nuclear pore complex protein Nup214 (UniProt: P35658), DNA-directed RNA polymerase II subunit RPB1, Myeloblastin (UniProt: P24158), Phosphatidylinositol 5-phosphate 4-kinase type-2 alpha (UniProt: P48426), RNA helicase (UniProt: A0A712V2S0), Cytochrome c oxidase subunit 2, RNA-binding protein NOB1 (UniProt: Q9ULX3), Dermatan-sulfate epimerase (UniProt: Q9UL01), Dermatan-sulfate epimerase (UniProt: A0A2R8YE23), Squamous cell carcinoma antigen recognized by T-cells 3 (UniProt: A0A499FI31), Squamous cell carcinoma antigen recognized by T-cells 3 (UniProt: Q15020), Peptidyl-prolyl cis-trans isomerase B, Melanoma-associated antigen 10, Secreted protein BARF1, Triosephosphate isomerase, 3 beta-hydroxysteroid dehydrogenase type 7, HLA class II histocompatibility antigen, DP alpha 1 chain, Calreticulin (UniProt: P27797), Minor capsid protein L2, Filamin-A (UniProt: P21333), Alpha-2-macroglobulin, Telomerase reverse transcriptase, Cytochrome b, HLA class II histocompatibility antigen, DR beta 5 chain, Chondroitin sulfate proteoglycan 4, Follistatin-related protein 1, Desmoglein-3, T cell receptor beta chain MC.7.G5, Regulatory protein E2, Major capsid protein L1, Cullin-associated NEDD8-dissociated protein 2, Major capsid protein L1, Transcriptional activator protein Pur-alpha, Importin-8, Probable G-protein coupled receptor 139, KICSTOR complex protein SZT2, ATP-citrate synthase, Heterogeneous nuclear ribonucleoprotein U (UniProt: Q5RI18), Hsp90 co-chaperone Cdc37-like 1 (UniProt: Q7L3B6), Protein E7, Protein E4, Regulatory protein E2, Probable protein E5, Regulatory protein E2, Minor capsid protein L2, Protein E6, Protein E6, 14-3-3 protein gamma, Polyadenylate-binding protein 1, Serine/threonine-protein kinase SMG1, Protein transport protein Sec24D (UniProt: 094855), Interferon-inducible double-stranded RNA-dependent protein kinase activator A, Signal transducer and activator of transcription 1-alpha/beta (UniProt: P42224), E3 ubiquitin-protein ligase TRIP12 (UniProt: Q14669), Chromodomain-helicase-DNA-binding protein 3, Bromodomain adjacent to zinc finger domain protein 2A (UniProt: Q9UIF9), 26S proteasome non-ATPase regulatory subunit 2, Baculoviral IAP repeat-containing protein 7, E3 ubiquitin-protein ligase TRIM68, Putative HTLV-1-related endogenous sequence, Caspase-8, SAM and SH3 domain-containing protein 1, IgGFc-binding protein, Phosphatidate cytidylyltransferase 1, WD40 repeat-containing protein SMU1, NAD-dependent protein deacetylase sirtuin-2 (UniProt: Q8IXJ6), Tubulin alpha-1B chain, Histone H2B, Histone H2B type 1-D, THO complex subunit 4 (UniProt: Q86V81), Neuroblast differentiation-associated protein AHNAK, ATP-binding cassette sub-family A member 2 (UniProt: Q9BZC7), Plexin domain-containing protein 2, Membrane-associated phosphatidylinositol transfer protein 1, Baculoviral IAP repeat-containing protein 5 (UniProt: 015392), cAMP-dependent protein kinase type II-alpha regulatory subunit, Transformation/transcription domain-associated protein (UniProt: H0Y4W2), Sodium/potassium-transporting ATPase subunit alpha-3 (UniProt: P13637), Heterogeneous nuclear ribonucleoprotein L-like (Fragment), Neuronal membrane glycoprotein M6-b (UniProt: Q13491), von Willebrand factor, Alpha-synuclein (UniProt: A0A669KB41), Transgelin-2, Periplakin (UniProt: 060437), O-phosphoseryl-tRNA (Sec) selenium transferase (UniProt: Q9HD40), Protein disulfide-isomerase A3 (UniProt: P30101), Nuclear pore membrane glycoprotein 210, Cleavage and polyadenylation specificity factor subunit 1, Protein PRRC1, Histone acetyltransferase p300, Sterol O-acyltransferase 1, Peroxiredoxin-5, mitochondrial, Histone H1.5, Pericentriolar material 1 protein (UniProt: Q15154), Plectin (UniProt: Q15149), Lysine—tRNA ligase, Trinucleotide repeat-containing gene 6A protein, Plasminogen-binding protein PgbA, Nucleolar RNA helicase 2, Enoyl-CoA delta isomerase 2 (UniProt: 075521), Cytochrome P450 2E1, Collagen alpha-1 (VII) chain, Alanine-tRNA ligase, cytoplasmic, Extracellular calcium-sensing receptor (UniProt: P41180), ELAV-like protein 3, Protein-arginine deiminase type-4, Lysine-specific demethylase 5C, Tubulin beta chain (UniProt: P07437), Heparan sulfate 2-O-sulfotransferase 1, Glycophorin-C, ATP-dependent RNA helicase DDX3X (UniProt: 000571), Unconventional myosin-Ig (UniProt: B0I1T2), Tubulin beta-8 chain, Probable ATP-dependent RNA helicase DDX47, Microtubule-actin cross-linking factor 1, isoforms 1/2/3/5 (UniProt: H3BPE1), Homeodomain-interacting protein kinase 1 (UniProt: Q86Z02), Zinc finger homeobox protein 3, 40S ribosomal protein S15 (UniProt: P62841), RAS protein activator like-3, Melanoma antigen preferentially expressed in tumors, Probable ubiquitin carboxyl-terminal hydrolase FAF-X, ATP-dependent RNA helicase DDX1 (UniProt: Q92499), Alpha-actinin-1 (UniProt: P12814), Tax1-binding protein 1 (UniProt: Q86VP1), Myeloperoxidase, Protein ORF28, Cyclin-dependent kinase inhibitor 2A (UniProt: K7ES20), High affinity cationic amino acid transporter 1, Mucin-16, Histone-lysine N-methyltransferase 2A, Circadian clock protein PASD1, Bromodomain and WD repeat-containing protein 1, Signal transducer and activator of transcription 5B, Rho-related GTP-binding protein RhoC, Transforming protein RhoA (UniProt: C9JX21), WD repeat-containing protein 75 (UniProt: Q8IWA0), Multiple C2 and transmembrane domain-containing protein 1, Kinesin-like protein KIF16B (UniProt: A0A1B0GTU3), Targeting protein for Xklp2, Titin (UniProt: A0A0A0MTS7), NBAS subunit of NRZ tethering complex, ORM1-like protein 1, Protein TASOR 2 (UniProt: Q5VWN6), Interleukin-4 receptor subunit alpha (UniProt: P24394), Legumain, Phosphoglycerate kinase 1, [Pyruvate dehydrogenase [acetyl-transferring]]-phosphatase 1, mitochondrial, Myristoylated alanine-rich C-kinase substrate, Heterogeneous nuclear ribonucleoprotein L (UniProt: P14866), Transmembrane glycoprotein NMB (UniProt: Q14956), 72 kDa type IV collagenase, Calreticulin (UniProt: K7EL50), Macrosialin, Fibromodulin (UniProt: Q06828), Lumican, Malate dehydrogenase, cytoplasmic (UniProt: P40925), Collagen alpha-2 (V) chain, Clusterin (UniProt: P10909), Trafficking protein particle complex subunit 1, ETS translocation variant 5, E3 ubiquitin-protein ligase Mdm2 (UniProt: Q00987), Nicotinate-nucleotide pyrophosphorylase [carboxylating] (UniProt: Q15274), Procollagen-lysine,2-oxoglutarate 5-dioxygenase 2 (UniProt: 000469), Golgi-specific brefeldin A-resistance guanine nucleotide exchange factor 1 (UniProt: Q92538), E3 ubiquitin-protein ligase RNF126, Abl interactor 2 (UniProt: Q9NYB9), Protein NLRC5, ATP-dependent DNA helicase DDX11 (UniProt: Q96FC9), E3 ubiquitin-protein ligase TRIM9, Tripartite motif-containing protein 67 (UniProt: Q6ZTA4), C—C motif chemokine 3, Receptor-type tyrosine-protein kinase FLT3, Mediator of RNA polymerase II transcription subunit 24, Ubiquitin carboxyl-terminal hydrolase (UniProt: G5E9A6), Vacuolar protein sorting-associated protein 13B (UniProt: Q7Z7G8), Protein SON (UniProt: P18583), Nuclear transcription factor Y subunit gamma (Fragment) (UniProt: Q5T6K5), E3 ubiquitin-protein ligase UBR1, Nuclear pore complex protein Nup107 (UniProt: P57740), Neurocan core protein, Replication termination factor 2 (UniProt: Q9BY42), Terminal uridylyltransferase 4 (UniProt: Q5TAX3), Hyaluronan mediated motility receptor, CDK5 regulatory subunit-associated protein 2 (UniProt: Q96SN8), T-complex protein 1 subunit zeta, ATP-dependent DNA helicase Q5, RNA polymerase II elongation factor ELL (UniProt: U3KQ90), Abnormal spindle-like microcephaly-associated protein (UniProt: Q8IZT6), SWI/SNF-related matrix-associated actin-dependent regulator of chromatin subfamily B member 1 (UniProt: Q12824), Putative tRNA (cytidine (32)/guanosine (34)-2′-O)-methyltransferase (UniProt: Q9UET6), Acyl-CoA 6-desaturase, 116 kDa U5 small nuclear ribonucleoprotein component, Nucleolar protein 56 (UniProt: 000567), Nuclear pore complex protein Nup98-Nup96 (UniProt: P52948), Breast cancer anti-estrogen resistance protein 3, Unconventional myosin-Ie (UniProt: Q12965), U3 small nucleolar RNA-associated protein 6 homolog, Negative elongation factor B (UniProt: Q8WX92), Protein mono-ADP-ribosyltransferase PARP4, DNA repair and recombination protein RAD54B (UniProt: Q9Y620), Condensin-2 complex subunit G2, Golgin subfamily B member 1, Bombesin receptor-activated protein C6orf89, Structural maintenance of chromosomes protein (UniProt: G8JLG1), Erbin (UniProt: Q96RT1), Transmembrane protein 161B (UniProt: B7Z6T3), E3 ubiquitin-protein ligase RNF213 (UniProt: Q63HN8), Neutral alpha-glucosidase AB (UniProt: Q14697), Neutral alpha-glucosidase AB (UniProt: E9PKU7), Equilibrative nucleobase transporter 1, Lengsin, Protein disulfide-isomerase A6, Nuclear pore complex protein Nup93 (UniProt: Q8N1F7), Homocysteine-responsive endoplasmic reticulum-resident ubiquitin-like domain member 1 protein, Short transient receptor potential channel 4-associated protein, Vacuolar protein sorting-associated protein 51 homolog, Mothers against decapentaplegic homolog 5 (SMAD5), Chromodomain-helicase-DNA-binding protein 4, Integrin beta-8, Nuclear pore complex protein Nup85 (UniProt: Q9BW27), Pre-mRNA-processing factor 6, Zinc finger protein 281, Pre-mRNA-processing-splicing factor 8, Nuclear pore complex protein Nup214 (UniProt: P35658), A-kinase anchor protein 13 (UniProt: Q12802), Midasin, Ras GTPase-activating protein-binding protein 2 (UniProt: Q9UN86), Cell division cycle protein 27 homolog (UniProt: P30260), Clathrin heavy chain 1, Solute carrier family 2, facilitated glucose transporter member 1, U3 small nucleolar RNA-associated protein 4 homolog, Dedicator of cytokinesis protein 7, Large subunit GTPase 1 homolog (UniProt: Q9H089), E3 ubiquitin-protein ligase HERC2, Talin-1, Transcription elongation factor SPT6, Threonine—tRNA ligase 1, cytoplasmic, Transmembrane emp24 domain-containing protein 4 (UniProt: Q7Z7H5), Eukaryotic initiation factor 4A-III, Structural maintenance of chromosomes protein 4, Splicing factor 3B subunit 4, Transcriptional regulator QRICH1, Tyrosine-protein kinase JAK2, Serine/threonine-protein kinase Nek8 (UniProt: Q86SG6), Guanine nucleotide-binding protein G(s) subunit alpha isoforms short (UniProt: P63092), Protein NipSnap homolog 2, Structural maintenance of chromosomes flexible hinge domain-containing protein 1, E3 ubiquitin-protein ligase MYLIP, Protein transport protein Sec31A (UniProt: 094979), Protein transport protein Sec31A (UniProt: D6RHZ5), Dysferlin, Pecanex-like protein 2, Transducin-like enhancer protein 2 (UniProt: Q04725), Transducin-like enhancer protein 4 (UniProt: Q04727), Gamma-interferon-inducible protein 16 (UniProt: Q16666), Mitochondrial fission regulator 2, Complement decay-accelerating factor (UniProt: H7BY55), Sterile alpha motif domain-containing protein 9-like, Nicotinamide phosphoribosyltransferase (UniProt: A0A7P0T8L3), Ankyrin repeat domain-containing protein 12, Signal-induced proliferation-associated 1-like protein 3, Spatacsin (UniProt: Q96JI7), Sacsin (UniProt: Q9NZJ4), Iron-responsive element-binding protein 2, E3 ubiquitin-protein ligase UBR4 (UniProt: Q5T4S7), Heterogeneous nuclear ribonucleoprotein Q (UniProt: A0A712V5Q6), Gamma-tubulin complex component (UniProt: F2Z2B9), Gamma-tubulin complex component 2 (UniProt: Q9BSJ2), E3 ubiquitin-protein ligase MYCBP2, Insulin-induced gene 1 protein, Insulin-induced gene 1 protein (Fragment), Nucleoprotein TPR, Fasciculation and elongation protein zeta-1, Phosphatidylinositol-4,5-bisphosphate 3-kinase (UniProt: A0A2R8Y2F6), Phosphatidylinositol 4,5-bisphosphate 3-kinase catalytic subunit alpha isoform, Pogo transposable element with KRAB domain (Fragment), Brefeldin A-inhibited guanine nucleotide-exchange protein 2 (UniProt: Q9Y6D5), Protein Mpv17 (UniProt: P39210), Septin-9, Ubiquitin-conjugating enzyme E2 variant 3 (UniProt: Q8IX04), Neurofibromin (UniProt: P21359), Dynein axonemal intermediate chain 3 (UniProt: Q8IWG1), Exportin-7 (UniProt: Q9UIA9), Cytosolic carboxypeptidase 1, T-complex protein 1 subunit delta, Mediator of RNA polymerase II transcription subunit 13 (UniProt: Q9UHV7), Tubulin-folding cofactor B (UniProt: Q99426), Ras GTPase-activating protein-binding protein 1 (UniProt: A0A712YQN9), Ras GTPase-activating protein-binding protein 1 (UniProt: Q13283), Protein THEM6, Cytoskeleton-associated protein 2, Protein arginine N-methyltransferase 3, Histone-binding protein RBBP4 (UniProt: Q09028), Cyclin-G-associated kinase, Ubiquitin carboxyl-terminal hydrolase 7, PALM2 and AKAP2 fusion (Fragment) (UniProt: C9JVY5), Inosine-5′-monophosphate dehydrogenase 2, Valine—tRNA ligase, mitochondrial, Growth arrest-specific protein 7, M-phase inducer phosphatase 3, 60S ribosomal export protein NMD3 (UniProt: C9JA08), Glycogen [starch] synthase, muscle, WD repeat- and FYVE domain-containing protein 4, DNA damage-binding protein 1 (UniProt: Q16531), Protein PRRC2B, Cytoplasmic FMR1-interacting protein 2, Cytoplasmic FMR1-interacting protein 1, Prohibitin (Fragment) (UniProt: E7ESE2), Muskelin (UniProt: Q9UL63), Lysine-specific demethylase 2B (UniProt: Q8NHM5), Mediator of RNA polymerase II transcription subunit 14, BTB/POZ domain-containing protein 2, Double-stranded RNA-specific editase 1, Transcription factor Dp-2, Histone-lysine N-methyltransferase 2C, Cell division cycle-associated 7-like protein, F-box/WD repeat-containing protein 11 (UniProt: Q9UKB1), Store-operated calcium entry-associated regulatory factor, Histone-lysine N-methyltransferase EZH2, Myeloid-associated differentiation marker, Double-stranded RNA-specific adenosine deaminase (UniProt: A0A3B3ISU1), ATPase family AAA domain-containing protein 2 (UniProt: Q6PL18), Programmed cell death protein 7, HTLV-1 basic zipper factor, GTPase KRas, Guanine nucleotide binding protein-like 3 (Nucleolar)-like, isoform CRA_b, Melanoma-associated antigen F1, Serine/threonine-protein kinase ATR, Dynein axonemal heavy chain 17, Inositol 1,4,5-trisphosphate receptor type 1, Probable E3 ubiquitin-protein ligase HERC1, Double-strand-break repair protein rad21 homolog, Intercellular adhesion molecule 2, Crooked neck-like protein 1 (UniProt: Q5JY65), Inactive rhomboid protein 2, Transcription factor ETV6, Vacuolar protein sorting-associated protein 16 homolog, TATA box-binding protein-associated factor RNA polymerase I subunit B, A-kinase anchor protein 9 (UniProt: Q99996), Ubiquitin carboxyl-terminal hydrolase 47, Polyadenylate-binding protein 3, Zinc finger protein 280C, COP9 signalosome complex subunit 2 (UniProt: P61201), Endonuclease G, mitochondrial, Ribosomal L1 domain-containing protein 1, MAX gene-associated protein, Mitochondrial tRNA methylthiotransferase CDK5RAP1, Probable helicase senataxin, Carbonic anhydrase, Chromodomain-helicase-DNA-binding protein 2 (UniProt: Q14647), G patch domain-containing protein 8, Utp12 domain-containing protein, Cytochrome c oxidase subunit 7A2, mitochondrial (UniProt: P14406), Heparan-alpha-glucosaminide N-acetyltransferase (UniProt: Q68CP4), Probable ATP-dependent RNA helicase DHX37, Coronin-7 (UniProt: P57737), CREB-binding protein, Polycomb protein EED (UniProt: O075530), Integrator complex subunit 13, Paired amphipathic helix protein Sin3a (UniProt: Q96ST3), Clathrin interactor 1, Ubiquitin-like modifier-activating enzyme 1 (UniProt: P22314), Protein tyrosine phosphatase receptor type C-associated protein, Cyclin-dependent kinase 6, Ran-binding protein 9, Integrin alpha-1, E3 ubiquitin-protein ligase BRE1B, Proliferation marker protein Ki-67, Chromosome-associated kinesin KIF4B, Breast cancer type 2 susceptibility protein (UniProt: P51587), Transforming acidic coiled-coil-containing protein 3 (UniProt: Q9Y6A5), eIF-2-alpha kinase activator GCN1, Transcriptional adapter 1, Reticulon-4 receptor (Fragment) (UniProt: H7C0V4), P2X purinoceptor (UniProt: K7EQ78), Gag protein, Aggrecan core protein (UniProt: A0A5K1VW97), ATP-binding cassette sub-family C member 12, B-lymphocyte antigen CD19, Retinoic acid receptor alpha (UniProt: P10276), G1/S-specific cyclin-D3 (UniProt: P30281), Mothers against decapentaplegic homolog 4, Mitogen-activated protein kinase (UniProt: K7EN18), E3 ubiquitin-protein ligase TRIM23, Glycerol-3-phosphate dehydrogenase, mitochondrial, Casein kinase I isoform epsilon (UniProt: P49674), Kinesin-like protein KIF2C, Speckle-type POZ protein (UniProt: O43791), U4/U6.U5 tri-snRNP-associated protein 2 (UniProt: Q53GS9), SWI/SNF-related matrix-associated actin-dependent regulator of chromatin subfamily A member 5, Kelch-like ECH-associated protein 1, Serine palmitoyltransferase 2 (Fragment), Protein mono-ADP-ribosyltransferase PARP3, Enhancer of filamentation 1 (UniProt: Q14511), Sister chromatid cohesion protein PDS5 homolog A, Beta-1,3-galactosyl-O-glycosyl-glycoprotein beta-1,6-N-acetylglucosaminyltransferase, General transcription factor 3C polypeptide 2 (UniProt: Q8WUA4), Myotubularin, Citron Rho-interacting kinase (UniProt: O14578), Poly [ADP-ribose] polymerase (UniProt: E9PNI7), Transport and Golgi organization protein 6 homolog, Protein transport protein Sec24A, MMS19 nucleotide excision repair protein homolog, Proteasome activator complex subunit 4 (UniProt: Q14997), Myocyte-specific enhancer factor 2B (UniProt: Q02080), Transient receptor potential cation channel subfamily M member 8 (UniProt: Q7Z2W7), Rho GTPase-activating protein 35, Dihydropyrimidinase-related protein 4 (UniProt: Q5T0Q6), Guanine nucleotide-binding protein subunit beta-5, Aurora kinase A, Serine/threonine-protein kinase B-raf, Adenomatous polyposis coli protein, Nck-associated protein 1, Mediator of RNA polymerase II transcription subunit 15 (UniProt: G3V1P5), DNA excision repair protein ERCC-6-like (UniProt: Q2NKX8), HIV Tat-specific factor 1, Negative elongation factor A (Fragment) (UniProt: H0Y3X6), Negative elongation factor A (UniProt: Q9H3P2), Cytochrome P450 7B1, Intraflagellar transport protein 80 homolog, CIZ1 protein, FAST kinase domain-containing protein 1, mitochondrial, Cytochrome c oxidase subunit 4 isoform 1, mitochondrial, ATP synthase subunit g, mitochondrial (UniProt: O75964), Dynein axonemal assembly factor 5 (UniProt: Q86Y56), Eukaryotic translation initiation factor 2 subunit 1 (Fragment), LisH domain-containing protein ARMC9 (UniProt: A0A804HK42), Exostosin-2 (UniProt: Q93063), Dynamin-binding protein (UniProt: Q6XZF7), Tripartite motif-containing protein 26, Protein kintoun, Perilipin-2 (UniProt: Q99541), Quinone oxidoreductase, Protein PAT1 homolog 1, Neurobeachin-like protein 2 (UniProt: A0A494C1V1), Ufm1-specific protease 2 (UniProt: Q9NUQ7), Werner syndrome ATP-dependent helicase, WD repeat-containing protein 46, DBF4-type zinc finger-containing protein 2 (UniProt: Q9HCK1), Cytochrome b-c1 complex subunit 7, Heparan-sulfate 6-O-sulfotransferase 1, E3 ubiquitin-protein ligase HUWE1, NADP-dependent malic enzyme, RNA polymerase II subunit A C-terminal domain phosphatase (Fragment) (UniProt: K7EJD2), Neural Wiskott-Aldrich syndrome protein, Cocaine esterase, Casein kinase I isoform alpha (UniProt: P48729), BCL-6 corepressor (UniProt: Q6W2J9), Putative Polycomb group protein ASXL3 (UniProt: Q9C0F0), Retinoblastoma-like protein 2, Eyes absent homolog 3, Protein enabled homolog (UniProt: Q8N8S7), Integrin beta-4, F-actin-capping protein subunit beta (UniProt: P47756), Endoplasmic reticulum resident protein 29, 4F2 cell-surface antigen heavy chain (UniProt: P08195), Transketolase (UniProt: P29401), Nuclear receptor corepressor 1 (UniProt: A0A088AWL3), RanBP-type and C3HC4-type zinc finger-containing protein 1 (UniProt: Q9BYM8), Cell cycle and apoptosis regulator protein 2 (UniProt: Q8N163), B-cell receptor-associated protein (Fragment) (UniProt: C9JQ75), E3 ubiquitin-protein ligase Praja-2, Guanylate-binding protein 1, Chromatin target of PRMT1 protein (UniProt: Q9Y3Y2), Engulfment and cell motility protein 2 (UniProt: Q96JJ3), F-box only protein 21 (UniProt: Q4G104), Cofilin-2 (UniProt: Q9Y281), Kinetochore protein Nuf2 (UniProt: Q9BZD4), Trans-3-hydroxy-L-proline dehydratase (UniProt: Q96EM0), Helicase with zinc finger domain 2, DNA (cytosine-5)-methyltransferase 3A, Mitogen-activated protein kinase kinase kinase kinase 4 (MAPK4) (UniProt: G5E948), Phospholipid-transporting ATPase IF (UniProt: Q9Y2G3), Coatomer subunit gamma-2, MAP kinase-interacting serine/threonine-protein kinase 1 (UniProt: A0A499FIS5), Nucleophosmin (UniProt: P06748), BTB/POZ domain-containing protein 7 (UniProt: Q9P203), Melanoma inhibitory activity protein 2 (UniProt: G3V599), Phosphatidylinositol 4,5-bisphosphate 3-kinase catalytic subunit delta isoform, E3 ubiquitin-protein ligase DTX3L, FK506-binding protein 15, PH domain leucine-rich repeat-containing protein phosphatase 1, RAD50-interacting protein 1, Leucine-rich repeat-containing protein 42, ATP-dependent DNA/RNA helicase DHX36, TRAF-type zinc finger domain-containing protein 1, ATP-dependent RNA helicase DDX19B, AT-rich interactive domain-containing protein 5B (UniProt: Q14865), Centrosomal protein of 164 kDa, Cell division control protein 42 homolog (UniProt: A0A590UJK8), Constitutive coactivator of peroxisome proliferator-activated receptor gamma (UniProt: Q96EK7), Transmembrane protein 214 (UniProt: Q6NUQ4), E3 ubiquitin-protein ligase RNF38, Protein farnesyltransferase subunit beta (UniProt: B4DL54), Chromosome transmission fidelity protein 18 homolog (UniProt: Q8WVB6), Glutathione S-transferase kappa, Arf-GAP with Rho-GAP domain, ANK repeat and PH domain-containing protein 1 (UniProt: Q96P48), Mini-chromosome maintenance complex-binding protein, E3 ubiquitin-protein ligase TRIP12 (UniProt: A0A6Q8PGG9), FAST kinase domain-containing protein 4, Serine/threonine-protein kinase WNK2 (UniProt: Q9Y3S1), Non-specific serine/threonine protein kinase (UniProt: F8W9F9), Structural maintenance of chromosomes protein 1B, Peroxiredoxin-6, SWI/SNF complex subunit SMARCC2 (UniProt: Q8TAQ2), UDP-glucose: glycoprotein glucosyltransferase 2 (UniProt: Q9NYU1), Phosphatidylinositol 4-phosphate 5-kinase type-1 gamma (UniProt: 060331), Myosin light chain kinase, smooth muscle, Endoplasmin (UniProt: A0A7P0TAY2), DmX-like protein 1 (UniProt: Q9Y485), Tyrosine-protein kinase Lck, Cyclin-dependent kinase 4 (UniProt: P11802), Cyclin-dependent kinase 13 (UniProt: Q14004), Protein FAM136A (UniProt: Q96C01), Germ cell-specific gene 1-like protein 2, Calpain-7, Zonadhesin, Fanconi anemia group M protein, Protein FAM3C, Alstrom syndrome protein 1 (UniProt: Q8TCU4), Cyclic AMP-dependent transcription factor ATF-6 beta (UniProt: Q99941), WD repeat and FYVE domain-containing protein 1, Tensin-1 (UniProt: Q9HBL0), Astrotactin-2 (UniProt: O75129), Methionine synthase reductase, PR domain zinc finger protein 10 (UniProt: Q9NQV6), G protein pathway suppressor 2, Matrilysin, Dual specificity protein phosphatase 3, Serine/threonine-protein kinase PAK 3, Damage-control phosphatase ARMT1, HEAT repeat-containing protein 1 (UniProt: Q9H583), ATP synthase F (0) complex subunit C2, mitochondrial, Bone marrow stromal antigen 2, Cell division cycle 5-like protein, Regulator of nonsense transcripts 3A, Apoptosis inhibitor 5 (UniProt: G3V1C3), Apoptosis inhibitor 5 (UniProt: Q9BZZ5), Apoptosis inhibitor 5 (Fragment), PHD and RING finger domain-containing protein 1 (UniProt: A0A0J9YWD5), 1-phosphatidylinositol 4,5-bisphosphate phosphodiesterase gamma-1, Fc receptor-like A (Fragment), Eukaryotic translation initiation factor 4E (UniProt: D6RBW1), DNA/RNA-binding protein KIN17, Fibronectin type III domain-containing protein 3B, WD repeat-containing protein 35 (UniProt: Q9P2L0), Guanine nucleotide-binding protein G (I)/G(S)/G (O) subunit gamma-5, Serine/threonine-protein kinase PLK3, Ectoderm-neural cortex protein 1, Bromodomain adjacent to zinc finger domain protein 1A, Neuroepithelial cell-transforming gene 1 protein, Calcium load-activated calcium channel (UniProt: Q9UM00), Peroxisomal multifunctional enzyme type 2 (UniProt: P51659), Pleckstrin homology domain-containing family F member 2, TBC1 domain family member 14 (UniProt: Q9P2M4), Tyrosine-protein phosphatase non-receptor type 12, Lysine-specific demethylase 6B, Histone-lysine N-methyltransferase 2D, Zinc finger protein 292 (UniProt: J3KNV1), Endoplasmic reticulum transmembrane helix translocase, Alpha-methylacyl-CoA racemase (UniProt: Q9UHK6), Receptor-type tyrosine-protein phosphatase zeta, Probable cation-transporting ATPase 13A4, RNA-binding motif protein, X-linked-like-3, Ankyrin repeat domain-containing protein 26 (UniProt: Q9UPS8), Fructose-bisphosphate aldolase C, Nuclear protein MDM1 (UniProt: Q8TC05), ER lumen protein-retaining receptor 3, AN1-type zinc finger protein 5, mRNA export factor RAE1, Zinc phosphodiesterase ELAC protein 2 (Fragment) (UniProt: E7ES68), Insulin-like growth factor 2 mRNA-binding protein 2 (UniProt: Q9Y6M1), Acidic leucine-rich nuclear phosphoprotein 32 family member A (UniProt: P39687), Centrosomal protein of 55 kDa, Solute carrier family 12 member 4 (UniProt: Q9UP95), Transforming acidic coiled-coil-containing protein 2 (UniProt: E9PBC6), Transforming acidic coiled-coil-containing protein 2 (UniProt: O95359), Basal cell adhesion molecule (UniProt: P50895), Alkylglycerone-phosphate synthase (UniProt: A0A2R8YEL0), Probable phospholipid-transporting ATPase IIA, KIF-binding protein (UniProt: Q96EK5), Nuclear valosin-containing protein-like (UniProt: O15381), Inactive histone-lysine N-methyltransferase 2E (UniProt: Q8IZD2), C-terminal-binding protein 1, Bromodomain adjacent to zinc finger domain protein 2B (UniProt: Q9UIF8), Tumor protein 63, Helicase SKI2W, Serine-protein kinase ATM (UniProt: Q13315), STAGA complex 65 subunit gamma, Endoplasmic reticulum aminopeptidase 1, NPC intracellular cholesterol transporter 1, Synaptojanin-2, Non-specific serine/threonine protein kinase (UniProt: A0A804HLI0), Kalirin (UniProt: O60229), Ras suppressor protein 1, Neuronal cell adhesion molecule (UniProt: C9JYY6), Neutrophil cytosol factor 2, Protein DENND6B, ATP-dependent RNA helicase DHX29 (UniProt: Q7Z478), NIF3-like protein 1, E3 ubiquitin-protein ligase ARIH1 (UniProt: Q9Y4X5), Ankyrin repeat domain-containing protein 36C (UniProt: Q5JPF3), RecQ-mediated genome instability protein 1, ATP-dependent Clp protease proteolytic subunit, mitochondrial, Cyclin-dependent kinase inhibitor 2A (UniProt: P42771), RUN and FYVE domain-containing protein 1, Signal peptidase complex catalytic subunit SEC11 (UniProt: H0YNG3), Regulatory-associated protein of mTOR, Phospholipid-transporting ATPase VD, Glutamine—fructose-6-phosphate aminotransferase [isomerizing] 2, Interferon regulatory factor 5, DmX-like protein 2 (UniProt: Q8TDJ6), Pecanex-like protein 3, Probable ATP-dependent RNA helicase DDX31, General transcription factor IIE subunit 1, Disintegrin and metalloproteinase domain-containing protein 17, Bone morphogenetic protein receptor type-1B, Multiple epidermal growth factor-like domains protein 8, Zinc finger BED domain-containing protein 4, Multiple inositol polyphosphate phosphatase 1, Solute carrier family 2, facilitated glucose transporter member 5, Tau-tubulin kinase 2 (UniProt: Q6IQ55), Kelch-like protein 29, Transcriptional enhancer factor TEF-3 (UniProt: A0A0A0MRF3), PRELI domain-containing protein 1, mitochondrial (UniProt: Q9Y255), DNA polymerase theta, G1/S-specific cyclin-D1 (UniProt: P24385), Glutathione peroxidase 1 (UniProt: P07203), Kallikrein-2, Brevican core protein, Plexin-B3, EH domain-containing protein 2, V-type proton ATPase subunit B, brain isoform, Cholinesterase, Ras-specific guanine nucleotide-releasing factor 1, TBC1 domain family member 22A (UniProt: Q8WUA7), Cadherin EGF LAG seven-pass G-type receptor 1 (UniProt: A0A618PRU0), Cadherin EGF LAG seven-pass G-type receptor 3, Cadherin EGF LAG seven-pass G-type receptor 1 (UniProt: Q9NYQ6), Tumor protein p53-inducible protein 11 (UniProt: U3KQ32), Vesicle-associated membrane protein 3 (UniProt: K7EKX0), ATP-binding cassette sub-family A member 6, Peroxiredoxin-4 (UniProt: Q13162), DmX-like protein 1 (UniProt: F5H269), Ras GTPase-activating protein nGAP, Ryanodine receptor 2 (UniProt: Q92736), Pro-cathepsin H (UniProt: A0A712V3T9), Baculoviral IAP repeat-containing protein 5 (UniProt: H3BLT4), Baculoviral IAP repeat-containing protein 5 (UniProt: A0A0B4JIS3), Interleukin-1 beta, Eukaryotic translation initiation factor 4E-binding protein 1, Melanoma-associated antigen C2, Thioredoxin-dependent peroxide reductase, mitochondrial, Endothelin receptor non-selective type, Actin-related protein 10 (UniProt: Q9NZ32), Protocadherin-8, Sterile alpha motif domain-containing protein 9, Ubiquitin carboxyl-terminal hydrolase 37, Cytochrome c oxidase assembly factor 3 homolog, mitochondrial, Interleukin-27 receptor subunit alpha, 3-oxoacyl-[acyl-carrier-protein] synthase, mitochondrial, Phosphomannomutase 2, Uncharacterized protein KIAA1958, Collagen alpha-1 (XVI) chain, Zinc finger protein ZXDC, ATPase family protein 2 homolog, Chondroitin sulfate synthase 2, Epsin-3, Centrosomal protein of 192 kDa (UniProt: Q8TEP8), Plasmanylethanolamine desaturase, Homeobox protein TGIF2, Zinc finger MYM-type protein 4, Uncharacterized protein C6orf132, DNA helicase B (UniProt: Q8NG08), Serine/threonine-protein kinase STK11, Runt-related transcription factor 1, Nucleoporin NDC1, Unconventional myosin-IXa (UniProt: B2RTY4), Arginase-1, Sterol-4-alpha-carboxylate 3-dehydrogenase, decarboxylating, Vacuolar protein sorting-associated protein 4B (UniProt: 075351), Syncytin-1, Ubiquitin-conjugating enzyme E2 D2, All-trans-retinol 13,14-reductase, 28S ribosomal protein S5, mitochondrial, 39S ribosomal protein L39, mitochondrial, Protein-cysteine N-palmitoyltransferase HHAT, Transforming growth factor-beta-induced protein ig-h3, Multidrug resistance-associated protein 1, Hepatocyte growth factor receptor, Potassium voltage-gated channel subfamily C member 3 (UniProt: E7ETH1), Sodium channel protein type 4 subunit alpha, Isocitrate dehydrogenase [NADP] cytoplasmic, Sister chromatid cohesion protein PDS5 homolog B, Protein kinase C theta type, Isocitrate dehydrogenase [NADP], mitochondrial, Extracellular sulfatase Sulf-2, Nuclear protein localization protein 4 homolog (UniProt: Q8TAT6), CUB and sushi domain-containing protein 1 (UniProt: E5RIG2), GPI inositol-deacylase (UniProt: Q75T13), Teneurin-3, Mitogen-activated protein kinase kinase kinase 2, Transmembrane channel-like protein 8, Serine/threonine-protein kinase MRCK alpha, Protein-serine O-palmitoleoyltransferase porcupine, Xaa-Pro aminopeptidase 1, Structure-specific endonuclease subunit SLX4, Kinesin-like protein KIF26B (Fragment), Neuroligin-4, Y-linked (UniProt: Q8NFZ3), Neuroligin-4, X-linked, P2Y purinoceptor 4, SLIT-ROBO Rho GTPase-activating protein 1, Breast cancer anti-estrogen resistance protein 1 (UniProt: A0A2R8Y5E4), Protein mago nashi homolog 2 (UniProt: Q96A72), Carbonic anhydrase 2, Inactive hydroxysteroid dehydrogenase-like protein 1, Protoporphyrinogen oxidase, Dephospho-CoA kinase domain-containing protein, Calcium uniporter protein, mitochondrial, Armadillo repeat-containing protein 7, Transmembrane protein 168, Protein MMS22-like, Mannosyl-oligosaccharide 1,2-alpha-mannosidase IB, Neuron navigator 3 (UniProt: Q8IVL0), Neuron navigator 2 (UniProt: A0A0A0MTL4), Neuron navigator 2 (UniProt: Q8IVL1), Dedicator of cytokinesis protein 3, Neuroblastoma breakpoint family member 11, Serine/threonine-protein phosphatase 4 regulatory subunit 1, Gamma-tubulin complex component 6 (UniProt: Q96RT7), Vacuolar protein sorting-associated protein 8 homolog (UniProt: Q8N3P4), Protein asteroid homolog 1, TRIO and F-actin-binding protein, Guanylate cyclase soluble subunit alpha-1, Cryptochrome-1, Adhesion G protein-coupled receptor A3, Apoptosis regulator BAX (UniProt: Q07812), Transmembrane protein 231, Transmembrane protein 14B (UniProt: Q9NUH8), L-fucose kinase, Phospholipid-transporting ATPase ABCA1 (UniProt: 095477), Mitochondrial inner membrane protein OXA1L (UniProt: Q15070), NACHT, LRR and PYD domains-containing protein 14, Serine-rich coiled-coil domain-containing protein 1, Glucoside xylosyltransferase 1, Rapamycin-insensitive companion of mTOR, Integrin alpha-9 (UniProt: Q13797), Adenylate kinase 7, Anoctamin-3, Nuclear exosome regulator NRDE2, Calcitonin gene-related peptide type 1 receptor, Golgin subfamily A member 2, Receptor-interacting serine/threonine-protein kinase 3, Krev interaction trapped protein 1, Protein argonaute-2, RWD domain-containing protein 3, 60S ribosomal protein L5 (UniProt: A0A2R8Y4A2), Protocadherin-11 X-linked, Cysteine and glycine-rich protein 2, Schlafen family member 12-like, Nebulin, Alpha-1,3-mannosyl-glycoprotein 2-beta-N-acetylglucosaminyltransferase, Aldehyde dehydrogenase family 16 member A1 (UniProt: Q8IZ83), A disintegrin and metalloproteinase with thrombospondin motifs 7, Unconventional myosin-Vb, MYCBP-associated protein (UniProt: A0A140VK87), Sodium/hydrogen exchanger (UniProt: A0A7I2V2B0), Mucin-4 (UniProt: A0A0G2JNL3), Zinc finger FYVE domain-containing protein 9, Probable ubiquitin carboxyl-terminal hydrolase MINDY-4, Cytoplasmic protein NCK1, [Pyruvate dehydrogenase (acetyl-transferring)] kinase isozyme 3, mitochondrial, Cyclin-A1, Alpha-2,8-sialyltransferase 8E, 28S ribosomal protein S10, mitochondrial, C—C chemokine receptor type 6, Ankyrin repeat and SAM domain-containing protein 1A (UniProt: Q92625), Kinesin-like protein KIF16B (UniProt: Q96L93), Dual specificity mitogen-activated protein kinase kinase 4, Adipocyte plasma membrane-associated protein, CD9 antigen (UniProt: P21926), Adhesion G-protein coupled receptor D1, Cytosolic iron-sulfur assembly component 3, Receptor-type tyrosine-protein phosphatase beta, RING finger protein 10, Myomegalin (UniProt: A0A075B749), NADH dehydrogenase [ubiquinone] flavoprotein 1, mitochondrial (UniProt: G3V015), Phosphatidylinositol 3,4,5-trisphosphate 3-phosphatase and dual-specificity protein phosphatase PTEN, Solute carrier family 27 member 3, Amiloride-sensitive sodium channel subunit beta, E3 ubiquitin-protein ligase HACE1, Proteinase-activated receptor 3, Metalloproteinase inhibitor 3, Short-chain specific acyl-CoA dehydrogenase, mitochondrial, POC1 centriolar protein homolog B (UniProt: Q8TC44), Exocyst complex component 8, ATP-binding cassette sub-family B member 6, PAX-interacting protein 1, Phospholipid scramblase 4, Protein FAM228B (UniProt: POC875), Ras-related protein Rab-39B, Alpha/beta hydrolase domain-containing protein 17B, FAST kinase domain-containing protein 2, mitochondrial, Protein FAM83G, SH3 and PX domain-containing protein 2A, Mannose-6-phosphate isomerase (UniProt: P34949), Triokinase/FMN cyclase, Tectonin beta-propeller repeat-containing protein 2, Immunoglobulin superfamily member 2, Signal-regulatory protein gamma, Segment polarity protein disheveled homolog DVL-2, Arf-GAP with SH3 domain, ANK repeat and PH domain-containing protein 1 (UniProt: Q9ULH1), EF-hand calcium-binding domain-containing protein 13, Phospholipid-transporting ATPase IK (UniProt: 060423), Splicing factor 3A subunit 2, Glutaminyl-tRNA synthetase (Fragment), Poly(ADP-ribose)glycohydrolase, Ephrin type-B receptor 1, Protrudin, Proline-rich protein 7, C-Jun-amino-terminal kinase-interacting protein 2, Unconventional myosin-Ib (UniProt: E9PDF6), Transcriptional enhancer factor TEF-1 (UniProt: P28347), Protocadherin gamma-C5, Zinc finger protein 407, Zinc finger protein 3, Pentraxin-4, Rho GTPase-activating protein 29 (UniProt: Q52LW3), Plasma membrane calcium-transporting ATPase 4, Neurosecretory protein VGF, Transcription factor MafF (Fragment), Cancer/testis antigen family 45 member A10, Coiled-coil and C2 domain-containing protein 1A, Cyclin-dependent kinase inhibitor 1, Mitochondrial chaperone BCS1, Serpin B3, Receptor protein-tyrosine kinase (UniProt: Q504U8), Receptor protein-tyrosine kinase (UniProt: E9PFD7), Neutral and basic amino acid transport protein rBAT (UniProt: Q07837), Protein FAM161A (Fragment), Leucine-rich repeat transmembrane protein CCDC168, Serpin B4, Epithelial discoidin domain-containing receptor 1, Serine/threonine-protein kinase 36, Collagen alpha-1 (XVIII) chain, DCN1-like protein 4 (UniProt: Q92564), Probable ATP-dependent RNA helicase DDX60-like (UniProt: A0A804HKC9), Glutamate receptor ionotropic, kainate 3, Myb-related protein B, NADH dehydrogenase [ubiquinone] iron-sulfur protein 2, mitochondrial, Threonine aspartase 1, Testin, Transcription factor SOX-10, Doublecortin and CaM kinase-like 2, isoform CRA_c, Elongation of very long chain fatty acids protein 2, Pappalysin-2, Forkhead box protein O1, Inactive tyrosine-protein kinase PRAG1, Splicing regulatory glutamine/lysine-rich protein 1 (UniProt: Q8WXA9), Beta-1,4-glucuronyltransferase 1, Xylosyl- and glucuronyltransferase LARGE1, SH3 domain-binding protein 2, Liprin-beta-1 (UniProt: Q86W92), 5-oxoprolinase, Cancer/testis antigen 2, Solute carrier family 52, riboflavin transporter, member 2, ATP-binding cassette sub-family C member 3, Dipeptidyl peptidase 2, ETS translocation variant 1 (UniProt: P50549), Putative glycosyltransferase ALG1-like, 55 kDa erythrocyte membrane protein, Protein dopey-2, N-terminal kinase-like protein (UniProt: Q96KG9), Proton myo-inositol cotransporter (UniProt: Q96QE2), Arf-GAP with GTPase, ANK repeat and PH domain-containing protein 3 (UniProt: Q96P47), Zinc finger protein 423, Phospholipid-transporting ATPase (UniProt: A0A5K1VW70), Roundabout homolog 1 (UniProt: Q9Y6N7), Protein spire homolog 1 (UniProt: Q08AE8), Transcription factor ETV7, Small G protein signaling modulator 3, Hyaluronidase-2, Autism susceptibility gene 2 protein (UniProt: Q8WXX7), Hormone-sensitive lipase, Protein FAM50B, Signal-regulatory protein delta (UniProt: Q9H106), Zinc finger protein 385C, Tumor necrosis factor receptor superfamily member 11A, Prickle-like protein 2 (UniProt: Q7Z3G6), Erythroblast transformation-specific transcription factor ERG variant 10, Synaptotagmin-like protein 4, Galactokinase (UniProt: P51570), Cytochrome c oxidase assembly protein COX18, mitochondrial, Tyrosine-protein kinase ABL2, Motile sperm domain-containing protein 1, Transmembrane protein KIAA1109 (Fragment) (UniProt: H7C0G8), Mitogen-activated protein kinase 13, Caspase recruitment domain-containing protein 16, Glutamate receptor 4, Receptor-type tyrosine-protein phosphatase U, Ankyrin repeat domain-containing protein 40, Transcription factor MafF, SEZ6L protein, KH domain-containing, RNA-binding, signal transduction-associated protein 2 (UniProt: Q5VWX1), DALR anticodon-binding domain-containing protein 3, Elongin-C (UniProt: Q15369), Isobutyryl-CoA dehydrogenase, mitochondrial, Centromere protein L, Transmembrane protease serine 6, WD repeat-containing protein 87 (UniProt: Q6ZQQ6), Supervillin (UniProt: 095425), Sushi, von Willebrand factor type A, EGF and pentraxin domain-containing protein 1 (UniProt: Q4LDE5), Protein BANP (UniProt: Q8N9N5), Derlin-2 (UniProt: Q9GZP9), Zinc finger protein 7 (UniProt: P17097), HLA class II histocompatibility antigen, DR alpha chain (UniProt: Q30118), Polycomb group RING finger protein 5, Toll-like receptor 3, Protocadherin-16, Mitochondrial 2-oxodicarboxylate carrier, STE20-like serine/threonine-protein kinase, TBC1 domain family member 2A, Spectrin alpha chain, erythrocytic 1, Cadherin-7 (UniProt: Q9ULB5), Contactin-3, Terminal nucleotidyltransferase 5D, Nuclear receptor subfamily 4 group A member 1, Laminin subunit alpha-5, Cystic fibrosis transmembrane conductance regulator, Hydrocephalus-inducing protein homolog, Alpha-parvin, Tubulin delta chain, Tenascin-R, Ankyrin and armadillo repeat-containing protein (UniProt: Q7Z5J8), MORC family CW-type zinc finger protein 1, KAT8 regulatory NSL complex subunit 1 (UniProt: A0A0G2JNB1), PWWP domain-containing protein 2A, UPF0687 protein C20orf27, SET-binding protein (UniProt: Q9Y6X0), DNA repair protein RAD51 homolog 2 (UniProt: 015315), Sodium/myo-inositol cotransporter 2, Pleckstrin homology domain-containing family A member 4, Neurofascin (UniProt: 094856), Dystrobrevin beta, Aromatic-L-amino-acid decarboxylase, Protein VAC14 homolog, Activin receptor type-2A, MAM and LDL-receptor class A domain-containing protein 1, Sodium- and chloride-dependent neutral and basic amino acid transporter B(0+), Unconventional myosin-XVIIIa (UniProt: A0A0D9SFK2), Membrane-spanning 4-domains subfamily A member 18 (UniProt: A0A499FJ34), Protein phosphatase Slingshot homolog 1, Dehydrogenase/reductase SDR family member 1, Eyes absent homolog 2, Importin-13 (UniProt: O94829), Golgi reassembly-stacking protein 2, DNA-dependent metalloprotease SPRTN, Enoyl-CoA hydratase, Protein WWC2, Probable asparagine—tRNA ligase, mitochondrial, Sodium/glucose cotransporter 5, Valyl-tRNA synthetase, Hamartin (UniProt: Q92574), Serine/threonine-protein kinase ULK1, Cyclin-dependent kinase-like 3 (UniProt: Q8IVW4), Protocadherin Fat 2, Histone-lysine N-methyltransferase 2B, Protein-tyrosine sulfotransferase 1, Suppressor of cytokine signaling 6, Slit homolog 3 protein (UniProt: O75094), 40S ribosomal protein S12, Cathepsin F (UniProt: Q9UBX1), Probable 2-oxoglutarate dehydrogenase E1 component DHKTD1, mitochondrial, Phosphorylase b kinase regulatory subunit alpha, skeletal muscle isoform, DENN domain-containing protein 2B (UniProt: P78524), ATP-binding cassette sub-family A member 2 (Fragment) (UniProt: H0Y8D6), Protein MTSS 2 (UniProt: Q765P7), Aldehyde dehydrogenase X, mitochondrial, Protein Niban 2, Protein FAM200A, Breast cancer anti-estrogen resistance protein 1 (UniProt: P56945), Amino acid transporter (UniProt: A0A087X0U3), Protein maestro (UniProt: Q9BYG7), Sodium- and chloride-dependent creatine transporter 1,1-phosphatidylinositol 4,5-bisphosphate phosphodiesterase delta-3, Constitutive coactivator of PPAR-gamma-like protein 2 (UniProt: F8W881), Caspase-5, Protein wntless homolog (UniProt: Q5T9L3), Androgen receptor (UniProt: P10275), Enoyl-CoA delta isomerase 2 (UniProt: A0A0C4DGA2), Acetyl-CoA carboxylase 2, AP-4 complex accessory subunit RUSC2, Protein odr-4 homolog, Nuclear receptor coactivator 6 (UniProt: Q14686), Neuroblastoma breakpoint family member 1 (UniProt: Q3BBV0), Rap1 GTPase-activating protein 2 (UniProt: A0A1B0GV05), Receptor expression-enhancing protein 5, Acid sphingomyelinase-like phosphodiesterase 3b, Junctophilin-2, Tyrosine-protein phosphatase non-receptor type 3, V-type proton ATPase 21 kDa proteolipid subunit c″, Neurogenic locus notch homolog protein 3, Trophoblast glycoprotein, Rap1 GTPase-activating protein 1 (UniProt: F2Z357), Ras-related protein Rab-1B (UniProt: E9PLD0), Inositol polyphosphate 5-phosphatase K, TGF-beta receptor type-2, Formin-2, Cytochrome c oxidase assembly factor 6 homolog, G-protein coupled receptor-associated sorting protein 2, Bcl-2-like protein 13 (UniProt: Q9BXK5), Myocyte-specific enhancer factor 2B (UniProt: B3KQ23), Protein FAM135B, Inactive tyrosine-protein kinase transmembrane receptor ROR1 (UniProt: Q01973), Methionine-R-sulfoxide reductase B2, mitochondrial, ENTPD4 protein, Endothelin-converting enzyme 2, EEF1AKMT4-ECE2 readthrough transcript protein, Homeobox protein Meis1 (UniProt: H0YNY8), BRD4-interacting chromatin-remodeling complex-associated protein-like, Ubiquinone biosynthesis O-methyltransferase, mitochondrial (UniProt: Q9NZJ6), Integrin beta-5, Frizzled-6 (UniProt: 060353), Specifically androgen-regulated gene protein, CTD nuclear envelope phosphatase 1, Growth arrest-specific protein 6, DNA-directed RNA polymerase III subunit RPC7-like, Exophilin-5, Fibroblast growth factor receptor 2, Coiled-coil domain-containing protein 120, Protein chibby homolog 2, Kallikrein-6, Villin-like protein (UniProt: 015195), Laminin subunit alpha-4 (UniProt: Q16363), SH3 and multiple ankyrin repeat domains protein 2 (UniProt: Q9UPX8), Diphosphoinositol polyphosphate phosphohydrolase 3-alpha, Centrosomal protein of 131 kDa (UniProt: Q9UPN4), Leucine zipper putative tumor suppressor 1, Olfactory receptor 2T3, HCG1811249, isoform CRA_e, Solute carrier family 25 member 48, Phosphodiesterase (UniProt: A1E5M1), Integrin alpha-7 (UniProt: Q13683), Gem-associated protein 2 (UniProt: 014893), Glutamate receptor ionotropic, NMDA 2B, Glypican-3, GPI mannosyltransferase 4, Mucin-4 (UniProt: Q99102), Mitochondrial peptide methionine sulfoxide reductase, Acyl-CoA synthetase short-chain family member 3, mitochondrial, UPF0692 protein C19orf54, Sushi repeat-containing protein SRPX, Galactocerebrosidase (UniProt: P54803), Transmembrane protein 260, Protein disulfide isomerase CRELD2, Arginine/serine-rich protein PNISR (UniProt: Q8TF01), Protein adenylyltransferase FICD, Zinc finger protein 785, Metalloreductase STEAP1, Mucolipin-2, Kynurenine formamidase (UniProt: K7EK09), Protein diaphanous homolog 1 (Fragment), SPARC, Cytospin-B (UniProt: A0A712YQJ3), Zinc finger protein 764, Serine protease hepsin, Olfactory receptor 5K3, Inactive phospholipase D5, Solute carrier family 22 member 9, Mucin-6 (UniProt: Q6W4X9), Transcription factor SOX-30, Killer cell immunoglobulin-like receptor 2DS4, Serine/threonine-protein kinase Nek10, HERV-H_2q24.3 provirus ancestral Env polyprotein, FH1/FH2 domain-containing protein 3 (Fragment), WD repeat-containing protein 91 (UniProt: A4D1P6), Cleavage and polyadenylation specificity factor subunit 6 (UniProt: Q16630), Transmembrane protein 104 (UniProt: Q8NE00), Programmed cell death 1 ligand 1, Protein ABHD12B, Aquaporin-7 (UniProt: B7Z4U2), Growth/differentiation factor 5, Photoreceptor cilium actin regulator, Membrane-associated guanylate kinase, WW and PDZ domain-containing protein 2 (UniProt: E7EWI0), Solute carrier family 5 member 4,1-phosphatidylinositol 4,5-bisphosphate phosphodiesterase beta-4, Vesicle-trafficking protein SEC22c (UniProt: Q9BRL7), GPNMB protein, Alpha-fetoprotein, Glycosaminoglycan xylosylkinase, Ras-related protein Rab-38, RING-type E3 ubiquitin transferase (UniProt: D6RAZ0), ATP-binding cassette sub-family B member 5, C-type lectin domain family 10 member A (UniProt: Q8IUN9), Complement component C7, Tissue factor, Adhesion G-protein coupled receptor G6, DNA excision repair protein ERCC-8 (UniProt: A0A6Q8PH55), Oligodendrocyte transcription factor 2, Lactadherin (Fragment), Anoctamin (UniProt: A0A2R8Y532), Angiotensin-converting enzyme (UniProt: F6X3S4), Lysosomal-associated transmembrane protein 4B (Fragment), Teashirt homolog 3, C-Jun-amino-terminal kinase-interacting protein 3 (UniProt: E9PFH7), Protogenin (UniProt: Q2VWP7), Ankyrin repeat domain-containing protein 20B, ATP-sensitive inward rectifier potassium channel 10, Sulfhydryl oxidase 2, Prickle planar cell polarity protein 3 (UniProt: 043900), Cyclin-Y-like protein 1, Transcription factor COE4 (UniProt: Q9BQW3), T-box transcription factor TBX4, Ribokinase, Protein phosphatase IF, Zinc finger protein GLIS3, Insulinoma-associated protein 2, DPCR1, Biorientation of chromosomes in cell division protein 1, Transmembrane protein 74B, E3 ubiquitin-protein ligase RNF149 (UniProt: F8WCD0), Choline transporter-like protein 5, ATP5MF-PTCDI readthrough, Semaphorin-3G, Olfactory receptor 5P2, Transient receptor potential cation channel subfamily M member 4, Putative protein FAM157B, G-protein-signaling modulator 1 (UniProt: Q86YR5), Patatin-like phospholipase domain-containing protein 4, Chloride channel protein C1C-Ka, Rho GTPase-activating protein 28 (UniProt: Q9P2N2), Mitochondrial thiamine pyrophosphate carrier, Leptin receptor, Zinc finger protein ZIC 4, Glypican-1 (UniProt: P35052), Olfactory receptor 4C6, Serpin 12, Mast/stem cell growth factor receptor Kit, Olfactory receptor 52N1, NLR family CARD domain-containing protein 4, E3 ubiquitin-protein ligase RNF43, NADPH oxidase 4 (UniProt: Q9NPH5), Phosphoinositide 3-kinase regulatory subunit 5 (UniProt: X6R3K3), PPP2R1A-PPP2R2A-interacting phosphatase regulator 1, Double zinc ribbon and ankyrin repeat-containing protein 1 (UniProt: Q9NVP4), Sodium-independent sulfate anion transporter, Glutamate receptor ionotropic, NMDA 3A, Two components: Latent membrane protein 2 & Latent membrane protein 2, Two components: Epstein-Barr nuclear antigen 3 & Epstein-Barr nuclear antigen 3, Two components: 65 kDa phosphoprotein & 65 kDa phosphoprotein, Two components: Latent membrane protein 1 & Latent membrane protein 1, Parathyroid hormone-related protein (Fragment), Oviduct-specific glycoprotein, Hepatitis A virus cellular receptor 2 (UniProt: Q8TDQ0), Anion exchange transporter, Cell adhesion molecule 2, Cadherin-24, Ubiquitin thioesterase OTU1, Acetylcholinesterase (UniProt: P22303), Olfactory receptor 2T4, Paraneoplastic antigen-like protein 8A, Malonyl-CoA-acyl carrier protein transacylase, mitochondrial, Calcium-binding mitochondrial carrier protein SCaMC-3 (Fragment), Netrin-1, Transmembrane protein 79 (UniProt: Q9BSE2), Potassium voltage-gated channel subfamily A member 10, Solute carrier family 22 member 16 (Fragment), Vang-like protein 1, Roundabout homolog 3 (UniProt: Q96MS0), Trypsin-3 (UniProt: P35030), Fragile X mental retardation 1 neighbor protein, T-cell surface glycoprotein CD1c, Potassium voltage-gated channel subfamily H member 1 (UniProt: 095259), Putative gap junction epsilon-1 protein, SLIT and NTRK-like protein 1, Uncharacterized protein KIAA0408, 5-hydroxytryptamine receptor 3D (UniProt: Q70Z44), Angiotensin-converting enzyme 2, Transcription factor SOX-2, Scavenger receptor cysteine-rich domain-containing group B protein, Lymphocyte antigen 6K, Embryonal Fyn-associated substrate, Regulator of G-protein signaling 5 (UniProt: 015539), A disintegrin and metalloproteinase with thrombospondin motifs 2 (UniProt: A0A1B0GTY3), Fatty acid-binding protein, brain, Cilia- and flagella-associated protein 65, Coiled-coil domain-containing protein 39 (UniProt: Q9UFE4), Zinc finger protein 521 (UniProt: Q96K83), Parathyroid hormone-related protein, Rho guanine nucleotide exchange factor 4 (UniProt: E7EV07), Leucine-rich repeat and fibronectin type-III domain-containing protein 3, Syntaxin-1A (UniProt: Q16623), Adenylate cyclase type 4, PWWP domain-containing DNA repair factor 3A (Fragment), IQ domain-containing protein F5, Sodium channel protein (UniProt: E9PG18), Oocyte-secreted protein 2, Rho guanine nucleotide exchange factor 10-like protein (UniProt: Q9HCE6), Trophinin (UniProt: A0A087X070), Short transient receptor potential channel 4, Sodium-dependent dopamine transporter, Unidentified protein, Brain-specific serine protease 4, Transmembrane protease serine 4 (UniProt: Q9NRS4), Choline O-acetyltransferase, Serine palmitoyltransferase small subunit B, Large T antigen, Ig gamma-2A chain C region, membrane-bound form, Podoplanin, Liprin-alpha-4 (UniProt: 075335), Signal peptide, CUB and EGF-like domain-containing protein 2, Claudin-18, Corticotropin-releasing factor-binding protein (UniProt: D6RHH7), Beta-1,4-N-acetylgalactosaminyltransferase 3, Solute carrier family 22 member 17 (UniProt: Q8WUG5), Armadillo repeat protein deleted in velo-cardio-facial syndrome (UniProt: E9PDC3), Selenoprotein V, Transmembrane protein 255A, Small T antigen ST, Nephrin, Titin (UniProt: Q8WZ42), ADAM9 protein, Retinitis pigmentosa 1-like 1 protein, Transmembrane and coiled-coil domain-containing protein 2, Protein crumbs homolog 1, Sorting nexin-16 (UniProt: A0A0C4DGW2), Distal membrane-arm assembly complex protein 2 (UniProt: B4DFT4), Olfactory receptor 4C11, PH and SEC7 domain-containing protein 1, Olfactory receptor 51E1, Contactin-6, COL11A2, POTE ankyrin domain family member H, Keratin, type I cytoskeletal 24, Uncharacterized serine/threonine-protein kinase SBK3, Arylsulfatase I, Tubulin polyglutamylase TTLL11, Aquaporin-12B, Calcitonin, Neuroblastoma breakpoint family member 14 (UniProt: A0A087WVU4), Inactive N-acetyllactosaminide alpha-1,3-galactosyltransferase (UniProt: Q4G0N0), Serine protease 33 (Fragment), 7-methylguanosine phosphate-specific 5′-nucleotidase (Fragment), Carcinoembryonic antigen-related cell adhesion molecule 18, Trypsin-3 (UniProt: A0A7P0MNE9), Leukocyte immunoglobulin-like receptor subfamily B member 3 (UniProt: A0A0G2JMM1), Uncharacterized protein C5orf60, Leucine-rich repeat-containing protein 37A3 (UniProt: 060309), Putative serpin A13, Coiled-coil domain-containing protein 74B (Fragment), Keratin-associated protein 11-1, Blood group Rh (CE) polypeptide (UniProt: E7EQ47), Protein SSX7, Melatonin-related receptor, Paired mesoderm homeobox protein 2A, Ankyrin repeat domain-containing protein 36B, Protein FAM83H (UniProt: A0A494C1T9), Homeobox protein Hox-A1 (UniProt: E7ERT8), Golgin subfamily A member 6-like protein 2 (UniProt: Q8N9W4), PRAME family member 4, PRAME family member 9, Leukocyte cell-derived chemotaxin-2, Proprotein convertase subtilisin/kexin type 5 (UniProt: B1AMG5), Olfactory receptor 5T3, Olfactory receptor 4C3, Phosphoinositide phospholipase C (UniProt: A0A3B3ISW9), Calpain-11 (Fragment), LILRA6, Family with sequence similarity 53, member A, isoform CRA_b, C-type lectin domain family 18 member C, Uncharacterized protein C9orf57, Homeobox protein Hox-B1, Two components: Insulin, isoform 2 & Endoplasmic reticulum chaperone BiP, Two components: Insulin (UniProt: P01308) & Endoplasmic reticulum chaperone BiP, Prostate stem cell antigen, Interleukin-22, BTB/POZ domain-containing protein 16, Olfactory receptor 51B4, Endogenous retrovirus group K member 18 Env polyprotein, U1tra-long-chain fatty acid omega-hydroxylase, Hyaluronan and proteoglycan link protein 3 (UniProt: H3BTH8), Kallikrein-4, Two components: Spike glycoprotein & Spike glycoprotein, Two components: Major capsid protein L1 & Major capsid protein L1, ADAMTS-like protein 1 (UniProt: A2A343), Putative uncharacterized protein GAFA-1, SMAD5 antisense gene protein 1, Putative uncharacterized protein encoded by LINC00615, B-lymphocyte antigen CD20 (UniProt: E9PKH8), Endogenous retrovirus group K member 113 Gag polyprotein, Endogenous retrovirus group K member 24 Gag polyprotein, Neuropeptides B/W receptor type 1, Cadherin-18 (UniProt: D6RIH8), SUMO-conjugating enzyme UBC9 (UniProt: B0QYN7), Neutral cholesterol ester hydrolase 1 (UniProt: A0A0A0MTJ9), Sodium/glucose cotransporter 4, Probable cysteine—tRNA ligase, mitochondrial (Fragment) (UniProt: F5H579), Uncharacterized protein (UniProt: A0A494C1I1), HCG2044781, P antigen family member 4, Two components: HLA-A*02:01 chain & Wilms tumor protein (UniProt: A0A0A0MT54), Two components: Immunoglobulin heavy constant gamma 1 & Immunoglobulin heavy constant gamma 1, Receptor tyrosine-protein kinase erbB-2, Two components: Genome polyprotein & Genome polyprotein, or Claudin-18.

In some embodiments, an extracellular domain can comprise one or more epitopes. In some embodiments, an extracellular domain can comprise one or more epitopes that are homologous to the extracellular domain. In some embodiments, extracellular domain can comprise one or more epitopes that are heterologous to the extracellular domain. In some embodiments, an extracellular domain can comprise from about 1 to about 10 epitopes. In some embodiments, an extracellular domain can comprise at least 1, 2, 3, 4, 5, 6, 7, 8, 9, or at least 10 epitopes. In some embodiments, an extracellular domain can comprise two or more copies of the same epitope. In some embodiments, an extracellular domain can comprise two or more epitopes, wherein each of the two or more epitopes bind to a same antibody, peptide hormone, or growth factor. For example, one or more copies of the same epitope sequence be inserted to an extracellular domain sequence that naturally comprises one copy of the same epitope sequence.

In some embodiments, an extracellular domain can comprise two or more epitopes, wherein each of the two or more epitopes are different epitopes. In some embodiments, each of the two or more epitopes can bind to a same antibody, peptide hormone, or growth factor. In some embodiments, each of the two or more epitopes can bind to a different antibody, peptide hormone, or growth factor. In some embodiments, an extracellular domain can comprise two or more epitopes, wherein the two or more epitopes can comprise a combination of the same and different epitopes. For example, an extracellular domain can comprise at least four epitopes, wherein two of the at least four epitopes bind to one antibody, peptide hormone, or growth factor and the other two of the at least four epitopes bind to another antibody, peptide hormone, or growth factor.

In some embodiments, an epitope can comprise about 15 to about 300 continuous residues of an extracellular domain of a protein described herein. In some embodiments, an epitope can comprise at least about 15, 16, 17, 18, 19, 20, 21, 22, 23, 24, 25, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290 or at least about 300 continuous residues of an extracellular domain of a protein described herein.

In some embodiments, an epitope can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% identity to any one of SEQ ID NOs: 4, 5, 6, 18, 19, 20, 64, 65, or a variant thereof. In some embodiments, an epitope can comprise a sequence having at least 80% sequence identity to any one of SEQ ID NOs: 4, 5, 6, 18, 19, 20, 64, 65, or a variant thereof. In some embodiments, an epitope can comprise a sequence having 100% sequence identity to SEQ ID NO: 4, 5, 6, 18, 19, 20, 64, 65, or a variant thereof.

In one embodiment, an epitope can be derived from DLL3 or a variant thereof. In this embodiment, a DLL3 epitope can comprise about 15 to about 260 contiguous residues of an extracellular domain of DLL3. In some embodiments, a DLL3 epitope can comprise at least about 15, 16, 17, 18, 19, 20, 21, 22, 23, 24, 25, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, or at least about 260 continuous residues of an extracellular domain of DLL3. In some embodiments, a DLL3 epitope can comprise amino acid residue 27-492 of DLL3. In some embodiments, a DLL3 epitope of can comprise amino acid residue 189-209 of DLL3. In some embodiments, a DLL3 epitope can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% identity to SEQ ID NO: 5, 6, 18, 19, 20, 64, 65, or a variant thereof. In some embodiments, a DLL3 epitope can comprise a sequence having at least 80% identity to SEQ ID NO: 5, or a variant thereof. In some embodiments, a DLL3 epitope can comprise a sequence having 100% sequence identity to SEQ ID NO: 5, 6, 18, 19, 20, 64, 65, or a variant thereof.

In one embodiment, an epitope can be derived from SSTR2 or a variant thereof. In this embodiment, an SSTR2 epitope can comprise about 15 to about 280 contiguous residues of an extracellular domain of SSTR2. In some embodiments, an SSTR2 epitope can have at least about 15, 16, 17, 18, 19, 20, 21, 22, 23, 24, 25, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or at least about 300 continuous residues of an extracellular domain of SSTR2. In some embodiments, an SSTR2 epitope can comprise amino acid residue 39-314 of SSTR2. In some embodiments, an SSTR2 epitope can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% identity to SEQ ID NO: 4, or a variant thereof. In some embodiments, an SSTR2 epitope can comprise a sequence having at least 80% identity to SEQ ID NO: 4, or a variant thereof. In some embodiments, an SSTR2 epitope can comprise a sequence having 100% sequence identity to SEQ ID NO: 4, or a variant thereof.

In one embodiment, an epitope can be derived from PSMA or a variant thereof. In this embodiment, a PSMA epitope can comprise about 15 to about 300 contiguous residues of an extracellular domain of PSMA. In some embodiments, a PSMA epitope can have at least about 15, 16, 17, 18, 19, 20, 21, 22, 23, 24, 25, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or at least about 300 continuous residues of an extracellular domain of PSMA.

In some embodiments, an epitope can bind to an antibody, peptide hormone, or growth factor with a Kd of less than or equal to 20 nM. For example, an epitope can bind to an antibody, peptide hormone, or growth factor with a Kd of less than or equal to 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, an epitope can bind to an antibody, peptide, or growth hormone with a Kd of less than or equal to 100 pM, 90 pM, 80 pM, 70 pM, 60 pM, 50 pM, 40 pM, 30 pM, 20 pM, 10 pM, 5 pM, or 1 pM.

In some embodiments, an epitope described herein can comprise an activatable epitope. In some embodiments, an activatable epitope can be preferentially accessible to a binding agent (e.g., an antibody, peptide hormone, or growth factor) in a physical microenvironment inside or outside of a cell affected by a disease or condition, compared to a physical microenvironment inside or outside of a cell not affected by a disease or condition. In some embodiments, an activatable epitope can be selectively available for binding in a tumor microenvironment. For example, an activatable epitope can be preferentially accessible to a binding agent in a physical microenvironment inside or outside of a tumor cell, compared to a physical microenvironment inside or outside of a non-tumor cell. In some embodiments, an activatable epitope can be preferentially accessible to a binding agent in a physical microenvironment inside or outside of a cell affected by an autoimmune disease or a neurodegenerative disease, compared to a physical microenvironment inside or outside of a cell not affected by an autoimmune disease or a neurodegenerative disease. In some embodiments, an activatable epitope can be activated via a mechanism, including, but not limited to, phosphorylation, glycosylation, methylation, ubiquitination, α-acetylation, lipidation, lipidation by glycosylphosphatidylinositol, amidation, sulfonation, oxidation, or any combinations thereof. For example, an epitope can be accessible to a binding agent when one or more amino acid residues on the epitope is phosphorylated, glycosylated, methylated, ubiquitinated, or α-acetylated. For example, an epitope can be accessible to a binding agent when one or more amino acid residues on the epitope is modified by lipidation, lipidation by glycosylphosphatidylinositol, amidation, sulfonation, or oxidation.

In some embodiments, an activatable epitope described herein can be accessible to a binding agent via conformational change of one or more pH-sensitive helices. In some embodiments, an extracellular domain comprising an activatable epitope described herein can further comprise a pH-sensitive helix. A pH-sensitive helix can be derived from a natural source or from a recombinant source. For example, a pH-sensitive helix can be derived from any protein comprising a pH-sensitive helix. For example, a pH-sensitive helix can be designed and engineered de novo by organizing one or more amino acid residues. In some embodiments, conformational changes of a pH-sensitive helix can be driven by organization of one or more histidine residues in hydrogen-bond networks in the pH-sensitive helix. In some embodiments, a pH-sensitive helix can associate and multimerize with one or more pH-sensitive helices at a physiological pH, or at pH>6.5. In some embodiments, a pH-sensitive helix can undergo a conformational change at low pH, for example, a pH<6.5. In some embodiments, a pH-sensitive helix can undergo a conformation change at low pH, via a mechanism comprising electrostatic and/or steric repulsion that builds up as the histidine residues in hydrogen-bond networks become protonated. Details of pH-sensitive helix designs and mechanisms are described in Boyken S E, et al. De novo design of tunable, pH-driven conformational changes. Science. 2019 May 17; 364 (6441): 658-664. doi: 10.1126/science.aav7897. PMID: 31097662; PMCID: PMC7072037.

In some embodiments, an extracellular domain comprising an activatable epitope described herein can further comprise from about 1 to about 10 copies of pH-sensitive helices. For example, an extracellular domain comprising an activatable epitope described herein can further comprise at least 1, 2, 3, 4, 5, 6, 7, 8, 9, or at least 10 copies of pH-sensitive helices. In some embodiments, an extracellular domain comprising an activatable epitope described herein can comprise at least two copies of pH-sensitive helices. In this embodiment, an activatable epitope can be flanked by at least two copies of pH-sensitive helices.

In some cases, a pH-sensitive helix can comprise a pH-sensitive multimerization domain. In some embodiments, a pH-sensitive helix can associate or multimerize with one or more pH-sensitive helices at a physical pH or at a pH higher than 6.0. In some embodiments, an epitope may not be accessible to a binding agent when a pH-sensitive helix associate or multimerize with one or more pH-sensitive helices. In some embodiments, a pH higher than 6.0 can comprise pH>6.1, pH>6.2, pH>6.3, pH>6.4, pH>6.5, pH>6.6, pH>6.7, pH>6.8, pH>6.9, pH>7.0, pH>7.1, pH>7.2, pH>7.3, pH>7.4, pH>7.5, pH>7.6, pH>7.7, pH>7.8, pH>7.9, pH>8.0, pH>8.5, pH>9.0, pH>9.5, or pH>10.0.

In some embodiments, a pH-sensitive helix can dissociate from other one or more pH-sensitive helices at a low pH. For example, a pH-sensitive helix can dissociate from other one or more pH-sensitive helices at a pH<6.0, pH<5.9, pH<5.8, pH<5.7, pH<5.6, pH<5.5, pH<5.4, pH<5.3, pH<5.2, pH<5.1, pH<5.0, pH<4.9, pH<4.8, pH<4.7, pH<4.6, pH<4.5, pH<4.4, pH<4.3, pH<4.2, pH<4.1, pH<4.0, pH<3.5, pH<3.0, pH<2.5, pH<2.0, pH<1.5, or pH<1.0. In some embodiments, an epitope can be accessible to a binding agent when a pH-sensitive helices dissociate from other one or more pH-sensitive helices.

In some cases, an extracellular domain comprising an activatable epitope and a pH-sensitive helix can be configured to bind to the pH-sensitive helix. In some embodiments, a pH-sensitive helix can bind to the extracellular domain comprising the pH-sensitive helix at a physical pH or a pH higher than 6.0. In some embodiments, an epitope may not be accessible to a binding agent when a pH-sensitive helix binds to the extracellular domain comprising the pH-sensitive helix. In some embodiments, a pH higher than 6.0 can comprise pH>6.1, pH>6.2, pH>6.3, pH>6.4, pH>6.5, pH>6.6, pH>6.7, pH>6.8, pH>6.9, pH>7.0, pH>7.1, pH>7.2, pH>7.3, pH>7.4, pH>7.5, pH>7.6, pH>7.7, pH>7.8, pH>7.9, pH>8.0, pH>8.5, pH>9.0, pH>9.5, or pH>10.0.

In some embodiments, a pH-sensitive helix can dissociate from the extracellular domain comprising the pH-sensitive helix at a low pH. For example, a pH-sensitive helix can dissociate from the extracellular domain comprising the pH-sensitive helix at a pH<6.0, pH<5.9, pH<5.8, pH<5.7, pH<5.6, pH<5.5, pH<5.4, pH<5.3, pH<5.2, pH<5.1, pH<5.0, pH<4.9, pH<4.8, pH<4.7, pH<4.6, pH<4.5, pH<4.4, pH<4.3, pH<4.2, pH<4.1, pH<4.0, pH<3.5, pH<3.0, pH<2.5, pH<2.0, pH<1.5, or pH<1.0. In some embodiments, an epitope can be accessible to a binding agent when a pH-sensitive helix dissociate from the extracellular domain comprising the pH-sensitive helix.

In some cases, an engineered polypeptide described herein can comprise an activatable epitope or a ligand binding site incorporated into a cell membrane. In this embodiment, an engineered polypeptide can further comprise one or more pH-sensitive helices in an extracellular domain, wherein each of the one or more pH-sensitive helices can comprise a multimerization domain. In some embodiments, one or more pH-sensitive helices can associate or multimerize at a physiological pH or a pH higher than 6.0. In this embodiment, an epitope or a ligand binding site may not be accessible to a binding agent when the one or more pH-sensitive helices associate or multimerize. In some embodiments, a pH higher than 6.0 can comprise pH>6.1, pH>6.2, pH>6.3, pH>6.4, pH>6.5, pH>6.6, pH>6.7, pH>6.8, pH>6.9, pH>7.0, pH>7.1, pH>7.2, pH>7.3, pH>7.4, pH>7.5, pH>7.6, pH>7.7, pH>7.8, pH>7.9, pH>8.0, pH>8.5, pH>9.0, pH>9.5, or pH>10.0.

In some embodiments, the one or more pH-sensitive helices can dissociate from each other at a low pH. In this embodiment, an epitope or a ligand binding site can be accessible to a binding agent when the one or more pH-sensitive helices dissociate at a low pH. In some embodiments, a low pH can comprise a pH<6.0, pH<5.9, pH<5.8, pH<5.7, pH<5.6, pH<5.5, pH<5.4, pH<5.3, pH<5.2, pH<5.1, pH<5.0, pH<4.9, pH<4.8, pH<4.7, pH<4.6, pH<4.5, pH<4.4, pH<4.3, pH<4.2, pH<4.1, pH<4.0, pH<3.5, pH<3.0, pH<2.5, pH<2.0, pH<1.5, or pH<1.0.

In some cases, an engineered polypeptide described herein can comprise a secretion signal. In some embodiments, an engineered polypeptide comprising a secretion signal may not comprise a signal peptide. For example, a secretion signal can replace a signal peptide in an engineered polypeptide. In some embodiments, an engineered polypeptide described herein can comprise a membrane binding domain. In some embodiments, an engineered polypeptide comprising a membrane binding domain may not comprise a transmembrane domain. For example, a membrane binding domain may replace a transmembrane domain in an engineered polypeptide. In some embodiments, an engineered polypeptide described herein can comprise a secretion signal and a membrane binding domain. In some embodiments, a membrane binding domain can comprise a pH-sensitive membrane-binding helix. In some embodiments, an engineered polypeptide comprising a secretion signal and a pH-sensitive membrane-binding helix can propagate to one or more neighboring cells. In one example, an engineered polypeptide comprising a secretion signal and a pH-sensitive membrane-binding helix can be secreted to an extracellular fluid or a physical microenvironment and may not attach to a cell when secreted in a non-acidic or non-tumor extracellular environment. In some embodiments, a non-acidic or non-tumor environment can have pH higher than 6.5, for example, pH>6.6, pH>6.7, pH>6.8, pH>6.9, pH>7.0, pH>7.1, pH>7.2, pH>7.3, pH>7.4, pH>7.5, pH>7.6, pH>7.7, pH>7.8, pH>7.9, pH>8.0, pH>8.5, pH>9.0, pH>9.5, or pH>10.0. In some embodiments, a non-acidic or non-tumor environment can have pH=7.2 In another example, an engineered polypeptide comprising a secretion signal and a pH-sensitive membrane-binding helix can be secreted to an extracellular fluid or a physical microenvironment and attach to cells including the secreting cell and neighboring cells when secreted at a pH level that can allow assembly of the membrane-binding helix. In some embodiments, a pH level that can allow assembly of the membrane-binding helix can comprise pH equal to or less than 6.5, for example, pH<6.4, pH<6.3, pH<6.2, pH<6.1, pH<6.0, pH<5.9, pH<5.8, pH<5.7, pH<5.6, pH<5.5. In some embodiments, a pH level that can allow assembly of the membrane-binding helix can comprise pH=6.5.

In some embodiments, a pH-sensitive helix can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% identity to any one of SEQ ID NOs: 1-3. In some embodiments, a pH-sensitive helix can comprise a sequence having at least 80% identity to any one of SEQ ID NOs: 1-3. In some embodiments, a pH-sensitive helix can comprise a sequence having 100% identity to any one of SEQ ID NOs: 1-3.

In some embodiments, an extracellular domain comprising an activatable epitope described herein can comprise a sequence according to [helix]-[helix]-[helix]-[epitope], wherein [helix] denotes a pH-sensitive helix; and [epitope] denotes an activatable epitope.

In some embodiments, an extracellular domain comprising an activatable epitope described herein can comprise a sequence according to [helix]-[linker]-[helix]-[linker]-[helix]-[linker]-[epitope], wherein [helix] denotes a pH-sensitive helix; [linker] denotes a linker; and [epitope] denotes an activatable epitope. In some embodiments, a linker can comprise a sequence (GGS)n (SEQ ID NO: 89) or (GGS)nFCYWKTCT (GGS)n (SEQ ID NO: 90), wherein n is an integer between 1 to 10. For example, n=1, n=2, n=3, n=4, n=5, n=6, n=7, n=8, n=9, or n=10.

In some embodiments, an extracellular domain comprising an activatable epitope described herein can comprise a sequence according to [helix]-[linker]-[helix]-[linker]-[helix]-[epitope], wherein [helix] denotes a pH-sensitive helix; [linker] denotes a linker; and [epitope] denotes an activatable epitope. In some embodiments, a linker can comprise a sequence (GGS)n (SEQ ID NO: 89) or RLCRPRSAPSRCGPGLRPCAP (SEQ ID NO: 91), wherein n is an integer between 1 to 10. For example, n=1, n=2, n=3, n=4, n=5, n=6, n=7, n=8, n=9, or n=10.

In some cases, an engineered polypeptide comprising an extracellular domain comprising an epitope, a transmembrane or a membrane affinity domain, and/or a hinge domain can further comprise a scaffold domain. In some embodiments, a scaffold domain can be located N-terminal or C-terminal to a hinge domain. In some embodiments, a scaffold domain can be located N-terminal or C-terminal to an epitope. In some embodiments, a scaffold domain can comprise an epitope. In some embodiments, a scaffold domain can be located N-terminal to a hinge domain and C-terminal to an epitope. In some embodiments, a scaffold domain can be located N-terminal to a hinge domain and N-terminal to an epitope. In some embodiments, a scaffold domain can comprise an epitope and can be located N-terminal to a hinge domain. In some embodiments, a scaffold domain can be located at an N-terminus of a mature polypeptide (e.g. after translation and cleavage of a secretion signal).

In some embodiments, a scaffold domain can comprise a heavy chain variable (VH) domain. In some embodiments, a scaffold domain may not comprise a light chain variable (VL) domain. In some embodiments, a scaffold domain can comprise a VH domain and does not comprise a VL domain. In some embodiments, a scaffold domain can comprise a single chain antibody (nanobody or VHH domain). In some embodiments, a nanobody domain can comprise a mutation in a complementarity determining region (CDR1), CDR2, or CDR3 of a VH domain. In some embodiments, a nanobody domain can comprise an inactivating mutation in a CDR1, CDR2, or CDR3 of a VH domain. In some embodiments, such an inactivating mutation can comprise swapped CDR residues, substituting at least 1, 2, 3, 4, 5, 6, 7, 8, 9, 10, 11, 12, 13, 14, 15, 16, 17, 18, 19, or 20 residues within the CDR regions to swap the charge of the substituted amino acid or swap a hydrophobic amino acid for a hydrophilic amino acid, or replacing one or more of the CDRs with (GGS)n or (GGGS)n (SEQ ID NO: 92).

In some embodiments, a scaffold domain can comprise an epitope inserted in a CDR1, CDR2, or CDR3 of a VH domain, VHH domain, or nanobody domain. In some embodiments, a scaffold domain can comprise one or more epitopes inserted in a CDR1, CDR2, or CDR3 of a VH domain. For example, a scaffold domain can comprise about 1, 2, 3, 4, 5, 6, 7, 8, 9, or 10 epitopes inserted in a CDR1, CDR2, or CDR3 of a VH domain. In some embodiments, a scaffold domain can comprise two or more epitopes inserted in a CDR1, CDR2, or CDR3 of a VH domain, wherein each of the two or more epitopes are the same epitopes binding to a same binding agent. In some embodiments, a scaffold domain can comprise two or more epitopes inserted in a CDR1, CDR2, or CDR3 of a VH domain, wherein each of the two or more epitopes are different epitopes binding to a same binding agent. In some embodiments, a scaffold domain can comprise two or more epitopes inserted in a CDR1, CDR2, or CDR3 of a VH domain, wherein each of the two or more epitopes are different epitopes binding to a different binding agent. In some embodiments, a scaffold domain can comprise two or more epitopes inserted in a CDR1, CDR2, or CDR3 of a VH domain, wherein the two or more epitopes comprise a combination of the same epitopes and different epitopes binding to a same binding agent. In some embodiments, a scaffold domain can comprise two or more epitopes inserted in a CDR1, CDR2, or CDR3 of a VH domain, wherein the two or more epitopes comprise a combination of the same epitopes binding to a same binding agent and different epitopes binding to a same or different binding agent.

In some embodiments, a scaffold domain can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to SEQ ID NO: 63, or a variant thereof. In some embodiments, a scaffold domain can comprise a sequence having at least 80% sequence identity to SEQ ID NO: 63, or a variant thereof. In some embodiments, a scaffold domain can comprise a sequence having 100% sequence identity to SEQ ID NO: 63, or a variant thereof.

### Transmembrane Domains Membrane Binding Domains

In some aspects, an engineered polypeptide described herein can further comprise a transmembrane domain or a membrane affinity domain capable of associating with an outer membrane of a cell. In some embodiments, an engineered polypeptide sequence can comprise a sequence for an extracellular domain sequence containing an epitope and a sequence for a transmembrane domain or a membrane affinity domain capable of associating with an outer membrane of a cell encoded by a single nucleic acid sequence. In some embodiments, an engineered polypeptide sequence can be designed to comprise a transmembrane domain or a membrane affinity domain capable of associating with an outer membrane of a cell that is heterologous to the extracellular domain of the engineered polypeptide.

In some embodiments, an engineered polypeptide described herein can comprise a transmembrane domain. In some embodiments, a transmembrane domain can comprise a membrane-spanning protein domain. In some embodiments, a transmembrane domain can comprise one or more hydrophobic amino acid residues. In some embodiments, a transmembrane domain can be derived from a protein comprising a single, single-pass, or single-span transmembrane α-helix domain. In some embodiments, a transmembrane domain can be derived from a protein comprising a multi, multi-pass, or multi-span transmembrane α-helix domains. In some embodiments, a transmembrane domain can be derived from a protein comprising a monotropic transmembrane α-helix domain. In some embodiments, a transmembrane domain can be derived from a protein comprising a bitopic transmembrane α-helix domain. In some embodiments, a transmembrane domain can be derived from a protein comprising polytopic transmembrane α-helical domains. In some embodiments, a transmembrane can be derived from a protein comprising polytopic transmembrane β-sheet domains. In some embodiments, a transmembrane domain can comprise a single-pass transmembrane domain. In some embodiments, a transmembrane domain can be derived from a glycosylphosphatidylinositol (GPI)-anchored protein. In some embodiments, a GPI anchor can comprise a posttranslational modification of a polypeptide with a glycolipid.

In some embodiments, a transmembrane domain can be derived from a natural source or from a recombinant source. For example, a transmembrane domain can be derived from any membrane-bound or transmembrane proteins. Non-limiting examples of a transmembrane can include a transmembrane domain derived from DLL3, PSMA, SSTR2, PD-L1, EGFR, CD28, CD4, CD8, CD8a, CD8b, ICOS, CD73, NKG2D, MUC16, ROR1, HER2, HER3, HER4, TCR gamma, TCR delta, CD3 epsilon, CD3 gamma, CD3 delta, or CD3 zeta. In some embodiments, a transmembrane domain can be derived from DLL3, PSMA, SSTR2, PD-L1, EGFR, CD28, CD4, CD8, CD8a, CD8b, ICOS, or CD73.

In some cases, other transmembrane domains can be identified based on sequence annotations from UniProtKB/Swiss-Prot. Alternatively or additionally, transmembrane regions can be detected from primary sequences using TMHMM, Memsat, Phobius and the hydrophobic moment plot method of Eisenberg et al. (Eisenberg et al. J Mol Biol. 1984 Oct. 15; 179 (1): 125-42. doi: 10.1016/0022-2836 (84) 90309-7, which is incorporated by reference herein in its entirety). Suitable transmembrane domains can include 2-5 residues on the N- and C-termini of the predicted helix to ensure maintenance of the same membrane surface interactions.

In some embodiments, a transmembrane domain can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a transmembrane domain of any one of SEQ ID NOs: 68-76. In some embodiments, a transmembrane domain can comprise a sequence having at least 80% sequence identity to a transmembrane domain of any one of SEQ ID NOs: 68-76. In some embodiments, a transmembrane domain can comprise a sequence having 100% sequence identity to a transmembrane domain of any one of SEQ ID NOs: 68-76.

In some embodiments, an engineered polypeptide described herein can comprise a membrane affinity domain capable of associating with an outer membrane of a cell. For example, an engineered polypeptide can comprise a domain that associates with an out membrane of a cell through a mechanism comprising, but not limited to, interaction by an amphipathic α-helix parallel to a membrane plane (in-plane membrane helix); interaction by a hydrophobic loop; interaction by a covalently bound membrane lipid; or ionic or electrostatic interaction with membrane lipids. In some embodiments, a membrane affinity domain can be derived from a protein comprising a monotropic α-helix domain. In come embodiments, an engineered polypeptide comprising a membrane affinity domain can be anchored to a membrane by integration into the bilayer of covalently bound lipids. In some embodiments, a membrane affinity domain capable of associating with an outer membrane of a cell can be attached to one side of the membrane (e.g., outer side of membrane) and may not span the whole membrane.

Non-limiting examples of a membrane affinity domain can include members of large homology domain families, including the Pleckstrin Homology (PH) domain superfamily; Fab1, YTOB, Vac1, EEA1 (FYVE) domains; Phagocyte Oxidase or Phox Homology (PX) domains; C1 and C2 domains (defined by homology with regions in protein kinase C); Epsin N-Terminal Homology (ENTH) domains; Band 4.1, Ezrin, Radixin, Moesin (FERM) domains; and Bin, Amphiphysin, Rvs (BAR) domains. In some embodiments, a membrane affinity domain can comprise one or more alpha helices (e.g., ENTH domains); a combination of one or more beta strands and one or more alpha helices (e.g., C1 domains, PX domains); a beta sandwich (e.g., C2 domains); a beta barrel capped at one end by an alpha helix (e.g., PH domains); or dual zinc finger modules (e.g., FYVE domains. Details of membrane affinity domains are described in Hurley J H. Membrane binding domains. Biochim Biophys Acta. 2006 August; 1761 (8): 805-11. doi: 10.1016/j.bbalip.2006.02.020. Epub 2006 Mar. 24. PMID: 16616874; PMCID: PMC2049088.

In some embodiments, a membrane affinity domain can comprise a helix domain derived from bacteriorhodopsin. A bacteriorhodopsin is an integral membrane archaeal protein comprising a hexagonal lattice comprising three identical protein chains, each comprising seven transmembrane alpha helices and one molecule of retinal. Details of bacteriorhodopsins are described in Woolf T B. Molecular dynamics of individual alpha-helices of bacteriorhodopsin in dimyristoyl phosphatidylcholine. I. Structure and dynamics. Biophys J. 1997 November; 73 (5): 2376-92. doi: 10.1016/S0006-3495 (97) 78267-5. PMID: 9370432; PMCID: PMC1181140.

In some embodiments, a membrane affinity domain can comprise a C1, C2, PH, FYVE, PX, or ENTH domain, or a transmembrane helix derived from bacteriorhodopsin. In some embodiments, a membrane affinity domain can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to any one of SEQ ID NOs: 77-81. In some embodiments, a membrane affinity domain can comprise a sequence having at least 80% sequence identity to any one of SEQ ID NOs: 77-81. In some embodiments, a membrane affinity domain can comprise a sequence having 100% sequence identity to any one of SEQ ID NOs: 77-81.

### Hinge Domains

In some aspects, an engineered polypeptide described herein can further comprise a hinge domain. For example, an engineered polypeptide can comprise an extracellularly-oriented polypeptide spacer domain that can comprise a hinge domain. In some embodiments, an extracellularly-oriented polypeptide spacer domain comprising a hinge domain can connect a transmembrane domain and an extracellular domain comprising an epitope. In some embodiments, an extracellularly-oriented polypeptide spacer domain comprising a hinge domain can connect a membrane affinity domain associating with an outer membrane of a cell and an extracellular domain comprising an epitope. For example, an engineered polypeptide described herein can comprise a n extracellularly-oriented polypeptide spacer domain comprising a hinge domain between a transmembrane domain or a membrane affinity domain and an extracellular domain comprising an epitope. In some embodiments, an engineered polypeptide sequence can comprise a sequence for an extracellular domain sequence containing an epitope, a sequence for a transmembrane domain or a membrane affinity domain capable of associating with an outer membrane of a cell, and a sequence for an extracellularly-oriented polypeptide spacer domain comprising a hinge domain encoded by a single nucleic acid sequence. In some embodiments, an engineered polypeptide sequence can be designed to comprise a transmembrane domain or a membrane affinity domain capable of associating with an outer membrane of a cell that is heterologous to the extracellular domain of the engineered polypeptide.

In some embodiments, a hinge domain can be derived from an Ig-superfamily receptor. For example, a hinge domain can be derived from CD4, IgG1, IgG2, IgG3, IgG4, or IgK, or any combination thereof. In some embodiments, a hinge domain can comprise a sequence (GGGS)n (SEQ ID NO: 92), wherein n is an any integer. In some embodiments, n is an integer between 1 and 10 or 1 and 20. For example, n=1, n=2, n=3, n=4, n=5, n=6, n=7, n=8, n=9, or n=10. In some embodiments, a hinge domain can be derived from CD4, CD8, CD8a, CD8b, CD28, or any combination thereof.

In some cases, other transmembrane domains can be identified based on sequence annotations from UniProtKB/Swiss-Prot, or by identifying regions with homology to a CD4, IgG1, IgG2, IgG3, IgG4, IgK, CD8, CD8a, CD8b, or CD28 hinge region. In some embodiments, a hinge domain can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a hinge sequence of any one of SEQ ID NOs: 56, 57, 58, 59, 60, 61, 62, 66, 67, or a variant thereof. In some embodiments, a hinge domain can comprise a sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a hinge sequence of any of the proteins described herein. In some embodiments, a hinge domain can comprise a sequence having at least 80% sequence identity to a hinge sequence of any one of SEQ ID NOs: 56, 57, 58, 59, 60, 61, 62, 66, 67, or a variant thereof. In some embodiments, a hinge domain can comprise a sequence having 100% sequence identity to a hinge sequence of any one of SEQ ID NOs: 56, 57, 58, 59, 60, 61, 62, 66, 67, or a variant thereof.

### Detection Methods Antibodies Treatment

In some cases, the methods described herein involve administering to a subject a composition comprising a nucleic acid or a vector described herein in conjunction with administering to said subject an antibody or antigen-binding fragment thereof, a protein ligand or functional fragment thereof, or a small molecule configured to bind said epitope.

The nucleic acid, vector, antibody or antigen binding fragment thereof, protein ligand, or small molecule can be administered by any suitable method such as intravenously, subcutaneously, intraventricularly, intrathecally, intracerebroventricularly, transdermally, intramuscularly, orally, inhalation, nasally, rectally, intratumorally, or proxi-tumorally to the subject. Proxi-tumorally may denote administration to the tissue within proximity of a tumor, or administration into a region that would be predicted to be accessible to the tumor via the lymphatic system (e.g. an adjoining lymph node). Intratumoral or proxi-tumoral approaches may involve the use of additional imaging techniques such as e.g. endoscopic ultrasonography (see e.g. Shirley et al. Gastroenterol Res Pract. 2013; 2013:207129) or via a bronchoscope (see e.g. Rojas-Solano et al. J Bronchology Interv Pulmonol. 2018 July; 25(3): 168-175). In some embodiments, the composition is administered into at least one of the cervical, epitrochlear, supraclavicular, cervical, axillary, mediastinal, supratrochlear, mesenteric, inguinal, femoral, or popliteal lymph nodes. In some cases, lymph-node based administration may serve as a method of centralized local delivery to a tissue region. In some cases, a composition including the nucleic acid, vector, antibody or antigen binding fragment thereof, protein ligand, or small molecule can be a pharmaceutically acceptable composition (e.g. one comprising a pharmaceutically acceptable carrier). In some cases, the composition can be configured for intravenous administration.

In some cases administering the nucleic acid or vector and administering the antibody or antigen-binding fragment thereof, protein ligand or functional fragment thereof, or small molecule configured to bind an epitope are separated by a sufficient time to allow for an epitope encoded by the nucleic acid or vector to express by cells of the subject. In some embodiments, this period of time includes at least about 8, 12, 16, 24, 36, 48, 60, 72, 84, or 96 hours.

In some cases, a non-invasive imaging method can be performed on the subject after an antibody or antigen-binding fragment thereof, protein ligand or functional fragment thereof, or small molecule configured to bind an epitope is administered to the subject. Such non-invasive imaging methods include MRI imaging, PET imaging, SPECT imaging, photoacoustic imaging, and bioluminescent imaging. Polypeptides detectable by MRI imaging include polypeptide contrast agents, such as ferritin (or mutants thereof, such as Pyrococcus furiosus ferritin mutants L55P, F57S, or F123S), or lanthanide-binding proteins (or engineered fusions thereof, such as the LBT-ubiquitin fusions described in Daughtry et al. ChemBioChem 2012, 13, 2567-2574). Synthetic biomarkers detectable by PET or SPECT imaging include the human sodium iodide symporter (e.g. in conjunction with administration of PET-active iodine/iodide isotopes, see e.g. Penheiter et al. Curr Gene Ther. 2012 February; 12 (1): 33-47), HSV-tk or mutants thereof such as HSV-sr39tk (e.g. in conjunction with administration of positron-labeled acycloguanosine or pyrimidine analog PET reporters such as [18F]FHBG, see Yaghoubi S S et al. Nat Protoc. 2006; 1(6):3069-75), and the dopamine D2 receptor or mutants thereof such as D2R80A or D2R194A (e.g. in conjunction with administration of positron-labeled D2 binders such as 3-(2′-[18F]-fluoroethyl)-spiperone). polypeptides detectable by photoacoustic imaging include the pigment-producing enzymes such as β-galactosidase (e.g. in combination with administration of X-gal) and tyrosinase, autofluorescent proteins (e.g. GFP, mCherry, or derivatives thereof), non-fluorescent GFP-like chromoproteins (e.g. aeCP597 and cjBlue and derivatives thereof), bacteriophytochrome-based near-infrared fluorescent proteins (e.g. IFP1.4, Wi-Phy, IFP1.4rev, IFP2.0, iRFP713, iRFP720, iRFP713/V256C, iRFP682, iRFP702, iRFP670, mIFP, iBlueberry, GAF-FP, BphP1-FP/C20S, or AphB variants), and reversibly photoswitchable proteins (e.g. Dronpa, Dronpa-M159T, and BphP1 or variants thereof). Polypeptides bioluminescent imaging include luciferases (e.g. in combination with administration of coelenterazines described herein), including Gaussia luciferases, Renilla luciferases, and Photinus luciferases (e.g. including the engineered Ppy RE8 and RE9 versions described in Branchini et al. Anal. Biochem. 396 (2010): 290-297).

In some cases, non-invasive imaging methods can involve detection with a radioisotope linked or associated with an antibody or antigen-binding fragment thereof, protein ligand or functional fragment thereof, or small molecule configured to bind an epitope. In some embodiments the radioisotope comprises a positron-emitting radioisotope, an alpha-emitting radioisotope, a beta-emitting radioisotope, or a gamma-emitting radioisotope. In some embodiments the radioisotope is a positron-emitting radioisotope and comprises 124I, 68Ga 11C, 13N, 15O, 18F, 68Ga, 64Cu, 52Mn, 55Co, 89Zr, 82Rb, or any combination thereof. In some embodiments, the radioisotope comprises an alpha-emitting radioisotope and comprises 225Ac, 211At, 227Th, 224Ra, or any combination thereof. In some embodiments, the radioisotope comprises a beta-emitting radioisotope and comprises 177Lu, 67Cu, 131I, 90Y, 89Sr, 186Re, 165Dy, 32P, 166Ho, 188Re, or any combination thereof. In some embodiments, the radioisotope is a gamma-emitting radioisotope and comprises 99mTc, 123I, or 131I.

In some cases non-invasive imaging methods can involve the use of a contrast agent linked to or associated with an antibody or antigen-binding fragment thereof, protein ligand or functional fragment thereof, or small molecule configured to bind an epitope, such as an iron oxide nanoparticle (IONP), a superparamagnetic iron platinum nanoparticle, manganese (II), or gadolinium (III).

In some cases, methods described herein can involve treatment with an antibody or antigen-binding fragment thereof, protein ligand or functional fragment thereof, or small molecule configured to bind an epitope linked or associated with a therapeutic agent. Such agents include a protein toxin, diphtheria toxin, glucagon-like peptide (GLP-1), a cytotoxic immunomodulatory protein, a Fas ligand, auristatin or an analog thereof, a maytansinoid, a calicheamicin, duocarmycin or an analog thereof, or doxorubicin or an analog thereof.

### Example Embodiments

In some aspects, the present disclosure provides for a method of detecting, imaging, or treating a diseased cell, comprising: administering to a subject a composition, wherein said composition induces expression of a biomarker preferentially in a diseased cell versus a non-diseased cell, wherein said biomarker comprises an extracellular receptor configured to bind an affinity reagent configured for detection or treatment of said diseased cell. In some aspects, the present disclosure provides for a method of detecting, imaging, or treating a diseased cell, comprising: administering to a subject a composition, wherein said composition induces expression of a biomarker preferentially in a diseased cell versus a non-diseased cell, wherein said biomarker comprises an extracellular receptor containing multiple epitopes configured to bind one or more affinity reagent configured for detection or treatment of said diseased cell. In some aspects, the present disclosure provides for a method of detecting a diseased cell, comprising: administering to a subject a composition, wherein said composition induces expression of a biomarker preferentially in a diseased cell versus a non-diseased cell, wherein said biomarker comprises an activatable epitope, wherein said activatable epitope is preferentially accessible to binding in a physical microenvironment inside or outside of said cell affected by said disease compared to a microenvironment inside or outside of a cell not affected by said disease. In some embodiments, said biomarker further comprises a pH-sensitive helix. In some embodiments, said activatable epitope is flanked by two copies of pH-sensitive helices. In some embodiments, said pH-sensitive helix comprises a sequence having at least 80% identity to any one of SEQ ID NOs: 1-3. In some embodiments, said biomarker comprises a sequence according to: [helix]-[linker]-[helix]-[linker]-[helix]-[linker]-[epitope] Wherein [helix] denotes said pH-sensitive helix; [linker] denotes a linker comprising the sequence (GGS)n or (GGS) nFCYWKTCT (GGS)n (SEQ ID NO: 93); and [epitope] denotes said epitope. In some embodiments, said biomarker comprises a sequence according to: [helix]-[linker]-[helix]-[linker]-[helix]-[epitope] Wherein [helix] denotes said pH-sensitive helix; [linker] denotes a linker comprising the sequence (GGS)n or RLCRPRSAPSRCGPGLRPCAP (SEQ ID NO: 91); and [epitope] denotes said epitope. In some aspects, the present disclosure provides for a method of detecting or treating a diseased cell, comprising: administering to a subject a composition, wherein said composition induces surface expression of a biomarker comprising an activatable epitope in a diseased cell preferentially over surface expression of said biomarker in non-diseased cells; wherein said activatable epitope is preferentially accessible to binding in a microenvironment specific to said diseased cell compared to a microenvironment of a cell not affected by said disease; wherein said biomarker comprises a membrane-resident polypeptide or wherein said biomarker comprises a polypeptide comprising a transmembrane domain. In some embodiments, the method further comprises administering to said subject an antibody or antigen-binding fragment thereof, a protein ligand or functional fragment thereof, or a small molecule configured to bind said epitope. In some embodiments, the method further comprises detecting binding of said antibody or antigen binding fragment thereof to said epitope. In some aspects, the present disclosure provides for a method of detecting or treating a diseased cell, comprising: (a) administering to a subject a composition, wherein said composition induces surface expression of a biomarker comprising one or more epitopes in a diseased cell preferentially over surface expression of said biomarker in non-diseased cells; and (b) administering to said subject an antibody or antigen-binding fragment thereof, a protein ligand or functional fragment thereof, or a small molecule configured to bind said epitope or epitopes. In some embodiments, the method further comprises detecting binding of said antibody or antigen binding fragment, protein ligand or functional fragment thereof, or small molecule to said epitope. In some embodiments, said epitope is an activatable epitope preferentially accessible to binding in a microenvironment specific to said diseased cell compared to an intracellular or extracellular microenvironment of a cell not affected by said disease. In some embodiments, said activatable epitope is activated via phosphorylation, glycosylation, methylation, ubiquitinylation, A-acetylation, lipidation (eg GPI), amidation, sulfonation, oxidation, or any combination thereof. In some embodiments, said biomarker further comprises a pH-sensitive helix. In some embodiments, said activatable epitope is flanked by two copies of pH-sensitive helices. In some embodiments, said pH-sensitive helix comprises a sequence having at least 80% identity to any one of SEQ ID NOs: 1-3. In some embodiments, said biomarker comprises a sequence according to: [helix]-[linker]-[helix]-[linker]-[helix]-[linker]-[epitope], wherein [helix] denotes said pH-sensitive helix; [linker] denotes a linker comprising the sequence (GGS)n or (GGS) nFCYWKTCT (GGS)n (SEQ ID NO: 93); and [epitope] denotes said epitope. In some embodiments, said biomarker comprises a sequence according to: [helix]-[linker]-[helix]-[linker]-[helix]-[epitope], wherein [helix] denotes said pH-sensitive helix; [linker] denotes a linker comprising the sequence (GGS)n or RLCRPRSAPSRCGPGLRPCAP (SEQ ID NO: 91); and [epitope] denotes said epitope. In some embodiments, said biomarker further comprises a protease cleavage site for a cancer-associated protease (e.g. a protease overexpressed in cancer cell versus a normal cell or a protease that preferentially displays catalytic activity in a cancer microenvironment) alongside an scFv configured to bind said activatable epitope. In some embodiments, said cancer-associated protease is uPA, EPCAM, an MMP, a cathepsin, a serine protease, a granzyme, a furin, a cysteine protease, an elastase, or a type II transmembrane serine protease, Kallikrein-related peptidases, ‘a disintegrin and metalloproteinase’ (ADAM), plasminogen activators (a serine protease), a caspase, or any combination thereof. In some embodiments, said antibody or antigen-binding fragment thereof further comprises a radioisotope. In some embodiments, said radioisotope comprises a positron-emitting radioisotope, an alpha-emitting radioisotope, a beta-emitting radioisotope, or 99mTc. In some embodiments, said radioisotope is a positron-emitting radioisotope and comprises ¹²⁴I, ⁶⁸Ga 11C, 13N, 15O, 18F, 68Ga, 64Cu, 52Mn, 55Co, 89Zr, 82Rb, or any combination thereof. In some embodiments, said radioisotope comprises an alpha-emitting radioisotope and comprises 225Ac, 211At, 227Th, 224Ra, or any combination thereof. In some embodiments, said radioisotope comprises a beta-emitting radioisotope and comprises 177Lu, 67Cu, 131I, ⁹⁰Y, 89Sr, 186Re, 165Dy, 32P, 166Ho, 188Re, or any combination thereof. In some embodiments, said biomarker further comprises an extracellular domain comprising said epitope. In some embodiments, said extracellular domain is derived from a membrane protein endogenous to said subject. In some embodiments, said extracellular domain is derived from DLL3, PSMA, SSTR2, CD8a, CD4, TGFR1, IGF1R, PD-L1, EGFR, CD73, IgK, or IL-6. In some embodiments, said biomarker comprises an extracellular domain comprising a sequence having at least 80% identity to any one of SEQ ID NOs: 4-9. In some embodiments, said biomarker further comprises a hinge domain derived from IgG4, IgG1, CD8, or CD28, or the sequence (GGGS)n (SEQ ID NO: 92). In some embodiments, said biomarker further comprises a transmembrane domain derived from DLL3, PSMA, SSTR2, PD-L1, EGFR, CD28, CD4, CD8a, ICOS, or CD73. In some embodiments, said biomarker further comprises an intracellular domain comprising a reporter protein or an affinity tag. In some embodiments, said biomarker further comprises an intracellular domain comprising a reporter protein, wherein said reporter protein is derived from GFP, a luciferase, Nluc, or Fluc, or any combination thereof. In some embodiments, said biomarker further comprises an intracellular domain comprising an affinity tag, wherein said affinity tag comprises an HA, FLAG, or MYC sequence, or any combination thereof. In some embodiments, said biomarker comprises more than one copy of said epitope. In some embodiments, said composition comprises a nucleic acid vector comprising a coding sequence for said biomarker. In some embodiments, said coding sequence comprises a promoter operably linked to a nucleotide sequence encoding the biomarker. In some embodiments, said promoter drives expression of said biomarker in said diseased cell preferentially relative to expression of said biomarker in a non-diseased cell in said subject. In some embodiments, said promoter is selected from the group consisting of a Survivin promoter (BIRC5), a CXCR4 promoter, an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransferase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11A1) promoter, a collagen type I alpha 1 chain (COL1A1) promoter, a cystatin SN (CST1) promoter, a denticleless E3 ubiquitin protein ligase homolog (DTL) promoter, a family with sequence similarity 111 member B (FAM111B) promoter, a forkhead box A1 (FOXA1) promoter, a kinesin family member 20A (KIF20A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2A member 1 (SCGB2A1) promoter, topoisomerase II alpha (TOP2A) promoter, a ubiquitin D (UBD) promoter, a ubiquitin conjugating enzyme E2 C (UBE2C), a USH1 protein network component harmonin (USH1C), a V-set domain containing T cell activation inhibitor 1 (VTCN1) promoter, a Hexokinase type II promoter, a TRPM4 promoter, a stromelysin 3 promoter, a surfactant protein A promoter, a secretory leukoprotease inhibitor promoter, a tyrosinase promoter, a stress-inducible grp78/BiP promoter, an interleukin-10 promoter, an α-B-crystallin/heat shock protein 27 promoter, an epidermal growth factor receptor promoter, a mucin-like glycoprotein promoter, an mts1 promoter, an NSE promoter, a somatostatin receptor promoter, a c-erbB-3 promoter, a c-erbB-2 promoter, a c-erbB4 promoter, a thyroglobulin promoter, an α-fetoprotein promoter, a villin promoter, an albumin promoter, a glycoprotein A33 promoter, the B cell-specific Moloney leukemia virus insertion site 1 promoter, a cyclooxygenase-2 promoter, a fibroblast growth factor promoter; a human epidermal growth factor receptor 2, a human telomerase reverse transcriptase promoter; a kinase domain insert containing receptor promoter; a rad51 recombinase promoter; TTF-1, an urokinase-type plasminogen activator receptor promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (UBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box M1 (FOXM1) promoter, a cell proliferation regulating inhibitor of protein phosphatase 2A (KIAA1524) promoter, a kinesin family member 2C (KIF2C) promoter, a karyopherin subunit alpha 2 (KPNA2) promoter, a MYB proto-oncogene like 2 (MYBL2) promoter, a NIMA related kinase 2 (NEK2) promoter, a RAN binding protein 1 (RANBP1) promoter, a small nuclear ribonucleoprotein polypeptides B and B1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17orf53) promoter, a chromobox 3 (CBX30) promoter, a cyclin B1 (CCNB1) promoter, a cyclin E1 (CCNE1) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL10A1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18A (KIF18A) promoter, a kinesin family member 4A (KIF4A) promoter, a kinesin family member C1 (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, a NDC80, kinetochore complex component (NDC80) promoter, a nudix hydrolase 1 (NUDT1) promoter, a ribonuclease H2 subunit A (RNASEH2A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP13) promoter, a trophinin associated protein (TROAP) promoter, a ubiquitin conjugating enzyme E2 C (UBE2C) promoter, a WD repeat and HMG-box DNA binding protein 1 (WDHD1) promoter, an alpha fetoprotein (AFP) promoter, a fragment thereof, any combination thereof, a chimeric promoter compiled from a plurality of elements from the aforementioned, or a fully synthetic promoter composed of tiled transcription-factor binding sites. In some embodiments, said vector is a recombinant viral vector. In some embodiments, said vector is a non-viral vector. In some embodiments, said non-viral vector is a nanoplasmid, a plasmid, a minicircle, a close-ended linear duplex (CELiD), or a doggybone DNA vector (dbDNA). In some embodiments, said composition further comprises a transfection agent. In some embodiments, said transfection agent is a linear or branched polyethylenimine, nanoparticle, lipophilic particle, solid nanoparticle, peptide, micelle, dendrimer, polymeric composition, hydrogel, synthetic or naturally derived exosome, virus-like particles, or any combination thereof. In some embodiments, said disease is a cancer, an autoimmune disease, or a neurodegenerative disease. In some embodiments, said disease is a cancer. In some embodiments, said cancer comprises Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, Uveal Melanoma, lip melanoma, spindle cell carcinoma, liposarcoma, nasal sarcoma, mammary adenocarcinoma, insulinoma, osteosarcoma, mast cell tumors, hemangiosarcoma, non-small cell lung carcinoma (NSCLC), marginal lymphoma, malignant melanoma, or chronic lymphocytic leukemia. In some embodiments, said detecting binding of said antibody or antigen binding fragment thereof to said epitope comprises a PET imaging method or another radioisotope-based imaging method.

In some aspects, the present disclosure provides for a nucleic acid comprising: a cancer-specific promoter (e.g. a promoter of a gene overexpressed in a cancer cell relative to a normal cell) operably linked to a nucleotide sequence encoding a membrane-resident polypeptide biomarker comprising an activatable epitope. In some embodiments, said activatable epitope is activated via phosphorylation, glycosylation, methylation, ubiquitinylation, A-acetylation, lipidation (eg GPI), amidation, sulfonation, oxidation, or any combination thereof. In some embodiments, said biomarker further comprises an extracellular domain comprising said epitope and a transmembrane domain. In some embodiments, said biomarker further comprises a pH-sensitive helix. In some embodiments, said activatable epitope is flanked by two copies of pH-sensitive helices. In some embodiments, said pH-sensitive helix comprises a sequence having at least 80% identity to any one of SEQ ID NOs: 1-3. In some embodiments, said biomarker comprises a sequence according to: [helix]-[linker]-[helix]-[linker]-[helix]-[linker]-[epitope] Wherein [helix] denotes said pH-sensitive helix; [linker] denotes a linker comprising the sequence (GGS)n or (GGS) nFCYWKTCT (GGS)n (SEQ ID NO: 93); and [epitope] denotes said epitope. In some embodiments, said biomarker comprises a sequence according to: [helix]-[linker]-[helix]-[linker]-[helix]-[epitope]

Wherein [helix] denotes said pH-sensitive helix; [linker] denotes a linker comprising the sequence (GGS)n or RLCRPRSAPSRCGPGLRPCAP (SEQ ID NO: 91); and [epitope] denotes said epitope. In some embodiments, said biomarker further comprises a protease cleavage site for a cancer-associated protease (e.g. a protease overexpressed in cancer cell versus a normal cell or a protease that preferentially displays catalytic activity in a cancer microenvironment) alongside an scFv configured to bind said activatable epitope. In some embodiments, said cancer-associated protease is uPA, EPCAM, an MMP, a cathepsin, a serine protease, a granzyme, a furin, a cysteine protease, an elastase, or a type II transmembrane serine protease, Kallikrein-related peptidases, ‘a disintegrin and metalloproteinase’ (ADAM), plasminogen activators (a serine protease), a caspase, or any combination thereof. In some embodiments, said extracellular domain is derived from a membrane protein endogenous to said subject. In some embodiments, said extracellular domain is derived from DLL3, PSMA, SSTR2, CD8a, CD4, TGFR1, IGF1R, PD-L1, EGFR, CD73, IgK, or IL-6. In some embodiments, said biomarker comprises an extracellular domain comprising a sequence having at least 80% identity to any one of SEQ ID NOs: 4-9. In some embodiments, said biomarker further comprises a hinge domain derived from IgG4, IgG1, CD8, or CD28, or the sequence (GGGS)n (SEQ ID NO: 92). In some embodiments, said biomarker further comprises a transmembrane domain derived from DLL3, PSMA, SSTR2, PD-L1, EGFR, CD28, CD4, CD8a, ICOS, or CD73. In some embodiments, said biomarker further comprises an intracellular domain comprising a reporter protein or an affinity tag. In some embodiments, said biomarker further comprises an intracellular domain comprising a reporter protein, wherein said reporter protein is derived from GFP, a luciferase, Nluc, or Fluc, or any combination thereof. In some embodiments, said biomarker further comprises an intracellular domain comprising an affinity tag, wherein said affinity tag comprises an HA, FLAG, or MYC sequence, or any combination thereof. In some embodiments, said promoter is selected from the group consisting of a Survivin promoter (BIRC5), a CXCR4 promoter, an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransferase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11A1) promoter, a collagen type I alpha 1 chain (COL1A1) promoter, a cystatin SN (CST1) promoter, a denticleless E3 ubiquitin protein ligase homolog (DTL) promoter, a family with sequence similarity 111 member B (FAM111B) promoter, a forkhead box A1 (FOXA1) promoter, a kinesin family member 20A (KIF20A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2A member 1 (SCGB2A1) promoter, topoisomerase II alpha (TOP2A) promoter, a ubiquitin D (UBD) promoter, a ubiquitin conjugating enzyme E2 C (UBE2C), a USH1 protein network component harmonin (USH1C), a V-set domain containing T cell activation inhibitor 1 (VTCN1) promoter, a Hexokinase type II promoter, a TRPM4 promoter, a stromelysin 3 promoter, a surfactant protein A promoter, a secretory leukoprotease inhibitor promoter, a tyrosinase promoter, a stress-inducible grp78/BiP promoter, an interleukin-10 promoter, an α-B-crystallin/heat shock protein 27 promoter, an epidermal growth factor receptor promoter, a mucin-like glycoprotein promoter, an mts1 promoter, an NSE promoter, a somatostatin receptor promoter, a c-erbB-3 promoter, a c-erbB-2 promoter, a c-erbB4 promoter, a thyroglobulin promoter, an α-fetoprotein promoter, a villin promoter, an albumin promoter, a glycoprotein A33 promoter, the B cell-specific Moloney leukemia virus insertion site 1 promoter, a cyclooxygenase-2 promoter, a fibroblast growth factor promoter; a human epidermal growth factor receptor 2, a human telomerase reverse transcriptase promoter; a kinase domain insert containing receptor promoter; a rad51 recombinase promoter; TTF-1, an urokinase-type plasminogen activator receptor promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (UBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box M1 (FOXM1) promoter, a cell proliferation regulating inhibitor of protein phosphatase 2A (KIAA1524) promoter, a kinesin family member 2C (KIF2C) promoter, a karyopherin subunit alpha 2 (KPNA2) promoter, a MYB proto-oncogene like 2 (MYBL2) promoter, a NIMA related kinase 2 (NEK2) promoter, a RAN binding protein 1 (RANBP1) promoter, a small nuclear ribonucleoprotein polypeptides B and B1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17orf53) promoter, a chromobox 3 (CBX30) promoter, a cyclin B1 (CCNB1) promoter, a cyclin E1 (CCNE1) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL10A1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18A (KIF18A) promoter, a kinesin family member 4A (KIF4A) promoter, a kinesin family member C1 (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, a NDC80, kinetochore complex component (NDC80) promoter, a nudix hydrolase 1 (NUDT1) promoter, a ribonuclease H2 subunit A (RNASEH2A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP13) promoter, a trophinin associated protein (TROAP) promoter, a ubiquitin conjugating enzyme E2 C (UBE2C) promoter, a WD repeat and HMG-box DNA binding protein 1 (WDHD1) promoter, an alpha fetoprotein (AFP) promoter, a fragment thereof, any combination thereof, a chimeric promoter compiled from a plurality of elements from the aforementioned, or a fully synthetic promoter composed of tiled transcription-factor binding sites. In some embodiments, said nucleic acid comprises RNA or a capped mRNA. In some embodiments, said RNA or said capped mRNA comprises a splice signal, a synthetic intron, an miRNA binding site, a synthetic stem-loop or tetraloop, or an aRNA domain.

In some aspects, the present disclosure provides for a vector comprising any of the nucleic acids described herein. In some embodiments, said vector is a recombinant viral vector. In some embodiments, vector is a non-viral vector. In some embodiments, said non-viral vector is a nanoplasmid, a plasmid, a minicircle, a close-ended linear duplex (CELiD), or a Doggybone DNA vector (dbDNA).

In some aspects, the present disclosure provides for a composition comprising any of the vectors described herein or any of the nucleic acids described herein and a transfection agent. In some embodiments, said transfection agent is a linear or branched polyethylenimine, nanoparticle, lipophilic particle, solid nanoparticle, peptide, micelle, dendrimer, polymeric composition, hydrogel, synthetic or naturally derived exosome, virus-like particles, or any combination thereof.

In some aspects the present disclosure provides for a cell comprising any of the nucleic acids or vectors described herein.

TABLE 1
Sequences of Genes and Components Described Herein

| SEQ ID | | | |
|---|---|---|---|
| NO: | DESCRIPTION | SOURCE | SEQUENCE |
| 1 | pH- | Synthetic | GSEYEIRKALEELKASTAELKRATASLRAITEELKKNPSEDALVEHN |
| | sensitive | sequence | RAIVEHNAIIVENNRIIAAVLELIVRAIK |
| | helix #1 | | |
| 2 | pH- | Synthetic | GSEYEIRKALEELKASTAELKRATASLRASTEELKKNPSEDALVENN |
| | sensitive | sequence | RLIVEHNAIIVENNRIIAAVLELIVRAIK |
| | helix #2 | | |
| 3 | pH- | Synthetic | GSEYEIRKALEELKAATAELKRATASLRAITEELKKNPSEDALVEHN |
| | sensitive | sequence | RAIVEHNAIIVEHNRIIAAVLELIVRAIK |
| | helix #3 | | |
| 4 | SSTR2 | Homo | DLTSNAVLTFIYFVVCIIGLCGNTLVIYVILRYAKMKTITNIYILNL |
| | epitope | sapiens | AIADELFMLGLPFLAMQVALVHWPFGKAICRVVMTVDGINQFTSIFC |
| | (residues | | LTVMSIDRYLAVVHPIKSAKWRRPRTAKMITMAVWGVSLLVILPIMI |
| | 39-314) | | YAGLRSNQWGRSSCTINWPGESGAWYTGFIIYTFILGFLVPLTIICL |
| | | | CYLFIIIKVKSSGIRVGSSKRKKSEKKVTRMVSIVVAVFIFCWLPFY |
| | | | IFNVSSVSMAISPTPALKGMFDFVVVLTYANSCANPILYAF |
| 5 | DLL3 | Homo | AGVFELQIHSFGPGPGPGAPRSPCSARLPCRLFFRVCLKPGLSEEAA |
| | epitope | sapiens | ESPCALGAALSARGPVYTEQPGAPAPDLPLPDGLLQVPFRDAWPGTF |
| | version 1 | | SFIIETWREELGDQIGGPAWSLLARVAGRRRLAAGGPWARDIQRAGA |
| | (residues | | WELRFSYRARCEPPAVGTACTRLCRPRSAPSRCGPGLRPCAPLEDEC |
| | 27-492) | | EAPLVCRAGCSPEHGFCEQPGECRCLEGWTGPLCTVPVSTSSCLSPR |
| | | | GPSSATTGCLVPGPGPCDGNPCANGGSCSETPRSFECTCPRGFYGLR |
| | | | CEVSGVTCADGPCFNGGLCVGGADPDSAYICHCPPGFQGSNCEKRVD |
| | | | RCSLQPCRNGGLCLDLGHALRCRCRAGFAGPRCEHDLDDCAGRACAN |
| | | | GGTCVEGGGAHRCSCALGFGGRDCRERADPCAARPCAHGGRCYAHFS |
| | | | GLVCACAPGYMGARCEFPVHPDGASALPAAPPGLRPGDPQRYL |
| 6 | DLL3 | Homo | RLCRPRSAPSRCGPGLRPCAP |
| | epitope | sapiens | |
| | version 2 | | |
| | (residues | | |
| | 189-209) | | |
| 7 | Biomarker | Synthetic | [Helix]-GGSGGS-[Helix]-GGSGGS-[Helix]-GGSGGS- |
| | design 1 | sequence | [SSTR2]; |
| | for | | Wherein [helix] denotes any of the pH-sensitive |
| | SSTR2 | | helices described herein and [SSTR2] denotes |
| | | | any of the SSTR2 domains described herein |
| 8 | Biomarker | Synthetic | [Helix]-GGSGGSFCYWKTCTGGSGGS-[Helix]-GGSGGS- |
| | design 2 | sequence | [Helix]-GGSGGS-[SSTR2]; |
| | for | | Wherein [helix] denotes any of the pH-sensitive |
| | SSTR2 | | helices described herein and [SSTR2] denotes |
| | | | any of the SSTR2 domains described herein |
| 9 | Biomarker | Synthetic | [Helix]-RLCRPRSAPSRCGPGLRPCAP-[Helix]-GGSGGS- |
| | design 1 | sequence | Helix- |
| | for DLL3 | | APLVCRAGCSPEHGFCEQPGECRCLEGWTGPLCTVPVSTSSCLSPRG |
| | | | PSSATTGCLVPGPGPCDGNPCANGGSCSETPRSFECTCPRGFYGLRC |
| | | | EVSGVTCADGPCFNGGLCVGGADPDSAYICHCPPGFQGSNCEKRVDR |
| | | | CSLQPCRNGGLCLDLGHALRCRCRAGFAGPRCEHDLDDCAGRACANG |
| | | | GTCVEGGGAHRCSCALGFGGRDCRERADPCAARPCAHGGRCYAHFSG |
| | | | LVCACAPGYMGARCEFPVHPDGASALPAAPPGLRPGDPQRYL |
| 10 | pH- | Synthetic | AAEQNPIYWARYADWLFTTPLLLLDLALLVDADEGT |
| | sensitive | sequence | |
| | peptide | | |
| | #1 | | |
| 11 | pH- | Synthetic | ACDDQNPWRAYLDLLFPTDTLLLDLLW |
| | sensitive | sequence | |
| | peptide | | |
| | #2 | | |
| 12 | pH- | Synthetic | ACDDQNPWRAYLKLLFPTKTLLLKLLW |
| | sensitive | sequence | |
| | peptide | | |
| | #3 | | |
| 13 | pH- | Synthetic | ACDDQNPWARYLDWLFPTDTLLLDL |
| | sensitive | sequence | |
| | peptide | | |
| | #4 | | |
| 14 | pH- | Synthetic | ACEEQNPWARYLEWLFPTETLLLEL |
| | sensitive | sequence | |
| | peptide | | |
| | #5 | | |
| 15 | pH- | Synthetic | ACEDQNPYWARYADWLFTTPLLLLDLALLVDG |
| | sensitive | sequence | |
| | peptide | | |
| | #6 | | |
| 16 | Generic | Synthetic | AX(Z)ₙXPWRAYLDLLFPTDTLLLDLLWA |
| | pH- | sequence | Wherein X indicates any amino acid residue, |
| | sensitive | | including lysine or cysteine, Z indicates any |
| | peptide | | amino acid residue, and n is between 1 and 10 |
| 17 | CD8a | Synthetic | GGTTTPAPRPPTPAPTIASQPLSLRPEACRPAAGGAVHTRGLDFACD |
| | hinge | sequence | |
| 18 | DLL3 | Synthetic | AGVFELQIHSFGPGPGPGAPRSPCSARLPCRLFFRVCLKPGLSEEAA |
| | epitope | sequence | ESPCALGAALSARGPVYTEQPGAPAPDLPLPDGLLQVPFRDAWPGTF |
| | version 3 | | SFIIETWREELGDQIGGPAWSLLARVAGRRRLAAGGPWARDIQRAGA |
| | | | WELRFSYRARCEPPAVGTACTRLCRPRSAPSRCGPGLRPCAPLEDEC |
| | | | EAPLVCRAGCSPEHGFCEQPGECRCLEGWTGPLCTVPVST |
| 19 | DLL3 | Synthetic | EPPAVGTACTRLCRPRSAPSRCGPGLRPCAPLEDECEAPLVCRAGCS |
| | epitope | sequence | PEHGFCEQPGECRCLEGWTGPLCTVPVSTGGEPPAVGTACTRLCRPR |
| | version 4 | | SAPSRCGPGLRPCAPLEDECEAPLVCRAGCSPEHGFCEQPGECRCLE |
| | | | GWTGPLCTVPVST |
| 20 | DLL3 | Synthetic | QVQLVESGGGLVQPGGSLRLSCAASGGSEGSGETFSLGWFRQAPGQG |
| | epitope | sequence | LEAVAAIASMGGLTYYADSVKGRFTISRDNSKNTLYLQMNSLRAEDT |
| | version 5 | | AVYYCAAVGDVFAELPSSHNFRYWGQGTLVTVSSRGREPPAVGTACT |
| | | | RLCRPRSAPSRCGPGLRPCAPLEDECEAPLVCRAGCSPEHGFCEQPG |
| | | | ECRCLEGWTGPLCTVPVST |
| 21 | mIgK- | Synthetic | METDTLLLWVLLLWVPGSTGDAGVFELQIHSFGPGPGPGAPRSPCSA |
| | PP007- | sequence | RLPCRLFFRVCLKPGLSEEAAESPCALGAALSARGPVYTEQPGAPAP |
| | WT | | DLPLPDGLLQVPFRDAWPGTFSFIIETWREELGDQIGGPAWSLLARV |
| | (underline = | | AGRRRLAAGGPWARDIQRAGAWELRFSYRARCEPPAVGTACTRLCRP |
| | optional | | RSAPSRCGPGLRPCAPLEDECEAPLVCRAGCSPEHGFCEQPGECRCL |
| | signal | | EGWTGPLCTVPVSTGGTTTPAPRPPTPAPTIASQPLSLRPEACRPAA |
| | peptide; | | GGAVHTRGLDFACDAAEQNPIYWARYADWLFTTPLLLLDLALLVDAD |
| | normal = | | EGT |
| | DLL3 | | |
| | reporter; | | |
| | italic = | | |
| | CD8a hinge; | | |
| | bold = ph- | | |
| | sensitive | | |
| | peptide) | | |
| 22 | mIgK- | Synthetic | METDTLLLWVLLLWVPGSTGDAGVFELQIHSFGPGPGPGAPRSPCSA |
| | PP007- | sequence | RLPCRLFFRVCLKPGLSEEAAESPCALGAALSARGPVYTEQPGAPAP |
| | Var3 | | DLPLPDGLLQVPFRDAWPGTFSFIIETWREELGDQIGGPAWSLLARV |
| | (underline = | | AGRRRLAAGGPWARDIQRAGAWELRFSYRARCEPPAVGTACTRLCRP |
| | optional | | RSAPSRCGPGLRPCAPLEDECEAPLVCRAGCSPEHGFCEQPGECRCL |
| | signal | | EGWTGPLCTVPVSTGGTTTPAPRPPTPAPTIASQPLSLRPEACRPAA |
| | peptide; | | GGAVHTRGLDFACDACDDQNPWRAYLDLLFPTDTLLLDLLW |
| | normal = | | |
| | DLL3 | | |
| | reporter; | | |
| | italic = | | |
| | CD8a hinge; | | |
| | bold = ph- | | |
| | sensitive | | |
| | peptide) | | |
| 23 | mIgK- | Synthetic | METDTLLLWVLLLWVPGSTGDAGVFELQIHSFGPGPGPGAPRSPCSA |
| | PP007- | sequence | RLPCRLFFRVCLKPGLSEEAAESPCALGAALSARGPVYTEQPGAPAP |
| | Var3dead | | DLPLPDGLLQVPFRDAWPGTFSFIIETWREELGDQIGGPAWSLLARV |
| | (underline = | | AGRRRLAAGGPWARDIQRAGAWELRFSYRARCEPPAVGTACTRLCRP |
| | optional | | RSAPSRCGPGLRPCAPLEDECEAPLVCRAGCSPEHGFCEQPGECRCL |
| | signal | | EGWTGPLCTVPVSTGGTTTPAPRPPTPAPTIASQPLSLRPEACRPAA |
| | peptide; | | GGAVHTRGLDFACDACDDQNPWRAYLKLLFPTKTLLLKLLW |
| | normal = | | |
| | DLL3 | | |
| | reporter; | | |
| | italic = | | |
| | CD8a hinge; | | |
| | bold = ph- | | |
| | sensitive | | |
| | peptide) | | |
| 24 | mIgK- | Synthetic | METDTLLLWVLLLWVPGSTGDAGVFELQIHSFGPGPGPGAPRSPCSA |
| | PP007- | sequence | RLPCRLFFRVCLKPGLSEEAAESPCALGAALSARGPVYTEQPGAPAP |
| | Var5 | | DLPLPDGLLQVPFRDAWPGTFSFIIETWREELGDQIGGPAWSLLARV |
| | (underline = | | AGRRRLAAGGPWARDIQRAGAWELRFSYRARCEPPAVGTACTRLCRP |
| | optional signal | | RSAPSRCGPGLRPCAPLEDECEAPLVCRAGCSPEHGFCEQPGECRCL |
| | peptide; | | EGWTGPLCTVPVSTGGTTTPAPRPPTPAPTIASQPLSLRPEACRPAA |
| | normal = | | GGAVHTRGLDFACDACDDQNPWARYLDWLFPTDTLLLDL |
| | DLL3 | | |
| | reporter; | | |
| | italic = | | |
| | CD8a hinge; | | |
| | bold = ph- | | |
| | sensitive | | |
| | peptide) | | |
| 25 | mIgK- | Synthetic | METDTLLLWVLLLWVPGSTGDAGVFELQIHSFGPGPGPGAPRSPCSA |
| | PP007- | sequence | RLPCRLFFRVCLKPGLSEEAAESPCALGAALSARGPVYTEQPGAPAP |
| | Var7 | | DLPLPDGLLQVPFRDAWPGTFSFIIETWREELGDQIGGPAWSLLARV |
| | (underline = | | AGRRRLAAGGPWARDIQRAGAWELRFSYRARCEPPAVGTACTRLCRP |
| | optional | | RSAPSRCGPGLRPCAPLEDECEAPLVCRAGCSPEHGFCEQPGECRCL |
| | signal | | EGWTGPLCTVPVSTGGTTTPAPRPPTPAPTIASQPLSLRPEACRPAA |
| | peptide; | | GGAVHTRGLDFACDACEEQNPWARYLEWLFPTETLLLEL |
| | normal = | | |
| | DLL3 | | |
| | reporter; | | |
| | italic = | | |
| | CD8a hinge; | | |
| | bold = ph- | | |
| | sensitive | | |
| | peptide) | | |
| 26 | mIgK- | Synthetic | METDTLLLWVLLLWVPGSTGDAGVFELQIHSFGPGPGPGAPRSPCSA |
| | PP007- | | RLPCRLFFRVCLKPGLSEEAAESPCALGAALSARGPVYTEQPGAPAP |
| | Var1 | | DLPLPDGLLQVPFRDAWPGTFSFIIETWREELGDQIGGPAWSLLARV |
| | (underline = | | AGRRRLAAGGPWARDIQRAGAWELRFSYRARCEPPAVGTACTRLCRP |
| | optional | | RSAPSRCGPGLRPCAPLEDECEAPLVCRAGCSPEHGFCEQPGECRCL |
| | signal | | EGWTGPLCTVPVSTGGTTTPAPRPPTPAPTIASQPLSLRPEACRPAA |
| | peptide; | | GGAVHTRGLDFACDACEDQNPYWARYADWLFTTPLLLLDLALLVDG |
| | normal = | | |
| | DLL3 | | |
| | reporter; | | |
| | italic = | | |
| | CD8a hinge; | | |
| | bold = ph- | | |
| | sensitive | | |
| | peptide) | | |
| 27 | CD8aSP- | Synthetic | MALPVTALLLPLALLLHAARPEPPAVGTACTRLCRPRSAPSRCGPGL |
| | 2xDLL3 | | RPCAPLEDECEAPLVCRAGCSPEHGFCEQPGECRCLEGWTGPLCTVP |
| | (DSL, E1)- | | VSTGGEPPAVGTACTRLCRPRSAPSRCGPGLRPCAPLEDECEAPLVC |
| | CD8aH, | | RAGCSPEHGFCEQPGECRCLEGWTGPLCTVPVSTGGTTTPAPRPPTP |
| | PP042 | | APTIASQPLSLRPEACRPAAGGAVHTRGLDFACD |
| | modified | | |
| | to remove | | |
| | CD28TM | | |
| | (underline = | | |
| | optional | | |
| | signal | | |
| | peptide; | | |
| | italic = | | |
| | CD8a hinge; | | |
| | normal = | | |
| | DLL3 | | |
| | reporter) | | |
| 28 | CD8aSP- | Synthetic | MALPVTALLLPLALLLHAARPQVQLVESGGGLVQPGGSLRLSCAASG |
| | Nb-DLL3 | | GSEGSGETFSLGWFRQAPGQGLEAVAAIASMGGLTYYADSVKGRFTI |
| | (DSL, E1)- | | SRDNSKNTLYLQMNSLRAEDTAVYYCAAVGDVFAELPSSHNFRYWGQ |
| | CD8aH, | | GTLVTVSSRGREPPAVGTACTRLCRPRSAPSRCGPGLRPCAPLEDEC |
| | PP046 | | EAPLVCRAGCSPEHGFCEQPGECRCLEGWTGPLCTVPVSTGGTTTPA |
| | modified | | PRPPTPAPTIASQPLSLRPEACRPAAGGAVHTRGLDFACD |
| | to remove | | |
| | CD28TM | | |
| | (underline = | | |
| | optional | | |
| | signal | | |
| | peptide; | | |
| | italic = | | |
| | CD8a hinge; | | |
| | normal = | | |
| | DLL3 | | |
| | reporter) | | |
| 29 | mIgK- | Synthetic | METDTLLLWVLLLWVPGSTGDMSKGEELFTGVVPILVELDGDVNGHK |
| | GFP- | | FSVSGEGEGDATYGKLTLKFICTTGKLPVPWPTLVTTLSYGVQCFSR |
| | Var3dead | | YPDHMKQHDFFKSAMPEGYVQERTIFFKDDGNYKTRAEVKFEGDTLV |
| | (underline = | | NRIELKGIDFKEDGNILGHKLEYNYNEHLVYIMADKQKNGTKAIFQV |
| | optional | | HHNIEDGSVQLADHYQQNTPIGDGPVLLPDNHYLHTQSALSKDPNEK |
| | signal | | RDHMVLLEFVTAAGITHGMDELYKGGTTTPAPRPPTPAPTIASQPLS |
| | peptide; | | LRPEACRPAAGGAVHTRGLDFACDACDDQNPWRAYLKLLFPTKTLLL |
| | normal = | | KLLW |
| | GFP | | |
| | reporter; | | |
| | italic = | | |
| | CD8a hinge; | | |
| | bold = ph- | | |
| | sensitive | | |
| | peptide) | | |
| 30 | mlgK- | Synthetic | METDTLLLWVLLLWVPGSTGDMSKGEELFTGVVPILVELDGDVNGHK |
| | GFP- | | FSVSGEGEGDATYGKLTLKFICTTGKLPVPWPTLVTTLSYGVQCFSR |
| | VAR3 | | YPDHMKQHDFFKSAMPEGYVQERTIFFKDDGNYKTRAEVKFEGDTLV |
| | (underline = | | NRIELKGIDFKEDGNILGHKLEYNYNEHLVYIMADKQKNGTKAIFQV |
| | optional | | HHNIEDGSVQLADHYQQNTPIGDGPVLLPDNHYLHTQSALSKDPNEK |
| | signal | | RDHMVLLEFVTAAGITHGMDELYKGGTTTPAPRPPTPAPTIASQPLS |
| | peptide; | | LRPEACRPAAGGAVHTRGLDFACDACDDQNPWRAYLDLLFPTDTLLL |
| | normal = | | DLLW |
| | GFP | | |
| | reporter; | | |
| | italic = | | |
| | CD8a hinge; | | |
| | bold = ph- | | |
| | sensitive | | |
| | peptide) | | |
| 31 | mIgK- | Synthetic | METDTLLLWVLLLWVPGSTGDMSKGEELFTGVVPILVELDGDVNGHK |
| | GFP-WT | | FSVSGEGEGDATYGKLTLKFICTTGKLPVPWPTLVTTLSYGVQCFSR |
| | (underline = | | YPDHMKQHDFFKSAMPEGYVQERTIFFKDDGNYKTRAEVKFEGDTLV |
| | optional | | NRIELKGIDFKEDGNILGHKLEYNYNEHLVYIMADKQKNGTKAIFQV |
| | signal | | HHNIEDGSVQLADHYQQNTPIGDGPVLLPDNHYLHTQSALSKDPNEK |
| | peptide; | | RDHMVLLEFVTAAGITHGMDELYKGGTTTPAPRPPTPAPTIASQPLS |
| | normal = | | LRPEACRPAAGGAVHTRGLDFACDAAEQNPIYWARYADWLFTTPLLL |
| | GFP | | LDLALLVDADEGT |
| | reporter; | | |
| | italic = | | |
| | CD8a hinge; | | |
| | bold = ph- | | |
| | sensitive | | |
| | peptide) | | |
| 32 | mIgK- | Human | METDTLLLWVLLLWVPGSTGD |
| | cleavable | | |
| | signal | | |
| | peptide | | |
| 33 | CD8a | Human | MALPVTALLLPLALLLHAARP |
| | cleavable | | |
| | signal | | |
| | peptide | | |
| 34 | DLL3vEGFR | Synthetic | MRPSGTAGAALLALLAALCPASRAAGVFELQIHSFGPGPGPGAPRSP |
| | (NP244)- | | CSARLPCRLFFRVCLKPGLSEEAAESPCALGAALSARGPVYTEQPGA |
| | (DLL3 | | PAPDLPLPDGLLQVPFRDAWPGTFSFIIETWREELGDQIGGPAWSLL |
| | portion | | ARVAGRRRLAAGGPWARDIQRAGAWELRFSYRARCEPPAVGTACTRL |
| | spans | | CRPRSAPSRCGPGLRPCAPLEDECEAPLVCRAGCSPEHGFCEQPGEC |
| | DLL3 | | RCLEGWTGPLCTVPVSTSSCLSPRGPSSATTGCLVPGPGPCDGNPCA |
| | residues | | NGGSCSETPRSFECTCPRGFYGLRCEVSGVTCADGPCFNGGLCVGGA |
| | 27-492) | | DPDSAYICHCPPGFQGSNCEKRVDRCSLQPCRNGGLCLDLGHALRCR |
| | (underline = | | CRAGFAGPRCEHDLDDCAGRACANGGTCVEGGGAHRCSCALGFGGRD |
| | optional | | CRERADPCAARPCAHGGRCYAHFSGLVCACAPGYMGARCEFPVHPDG |
| | signal | | ASALPAAPPGLRPGDPQRYLSIATGMVGALLLLLVVALGIGLFMRRR |
| | peptide, | | HIVRKHMGGGSGGGSAYPYDVPDYAAYPYDVPDYA |
| | bold = DLL3 | | |
| | region, | | |
| | italics = | | |
| | transmembrane | | |
| | helix; | | |
| | double | | |
| | underline = | | |
| | optional | | |
| | affinity | | |
| | tag) | | |
| 35 | DLL3vPDL1 | Synthetic | MRIFAVFIFMTYWHLLNAAGVFELQIHSFGPGPGPGAPRSPCSARLP |
| | (NP245)- | | CRLFFRVCLKPGLSEEAAESPCALGAALSARGPVYTEQPGAPAPDLP |
| | DLL3 | | LPDGLLQVPFRDAWPGTFSFIIETWREELGDQIGGPAWSLLARVAGR |
| | portion | | RRLAAGGPWARDIQRAGAWELRFSYRARCEPPAVGTACTRLCRPRSA |
| | spans | | PSRCGPGLRPCAPLEDECEAPLVCRAGCSPEHGFCEQPGECRCLEGW |
| | DLL3 | | TGPLCTVPVSTSSCLSPRGPSSATTGCLVPGPGPCDGNPCANGGSCS |
| | residues | | ETPRSFECTCPRGFYGLRCEVSGVTCADGPCFNGGLCVGGADPDSAY |
| | 27-492 | | ICHCPPGFQGSNCEKRVDRCSLQPCRNGGLCLDLGHALRCRCRAGFA |
| | (underline = | | GPRCEHDLDDCAGRACANGGTCVEGGGAHRCSCALGFGGRDCRERAD |
| | optional | | PCAARPCAHGGRCYAHFSGLVCACAPGYMGARCEFPVHPDGASALPA |
| | signal | | APPGLRPGDPQRYLLPLAHPPNERTHLVILGAILLCLGVALTFIFRH |
| | peptide, | | MGGGSGGGSAYPYDVPDYAAYPYDVPDYA |
| | bold = DLL | | |
| | 3 region, | | |
| | italics = | | |
| | transmembrane | | |
| | helix; | | |
| | double | | |
| | underline = | | |
| | optional | | |
| | affinity | | |
| | tag) | | |
| 36 | CD8aSP- | Synthetic | MALPVTALLLPLALLLHAARPAGVFELQIHSFGPGPGPGAPRSPCSA |
| | DLL3- | | RLPCRLFFRVCLKPGLSEEAAESPCALGAALSARGPVYTEQPGAPAP |
| | CD28TM | | DLPLPDGLLQVPFRDAWPGTFSFIIETWREELGDQIGGPAWSLLARV |
| | (NP246)- | | AGRRRLAAGGPWARDIQRAGAWELRFSYRARCEPPAVGTACTRLCRP |
| | DLL3 portion | | RSAPSRCGPGLRPCAPLEDECEAPLVCRAGCSPEHGFCEQPGECRCL |
| | spans DLL3 | | EGWTGPLCTVPVSTSSCLSPRGPSSATTGCLVPGPGPCDGNPCANGG |
| | residues | | SCSETPRSFECTCPRGFYGLRCEVSGVTCADGPCFNGGLCVGGADPD |
| | 27-492 | | SAYICHCPPGFQGSNCEKRVDRCSLQPCRNGGLCLDLGHALRCRCRA |
| | (underline = | | GFAGPRCEHDLDDCAGRACANGGTCVEGGGAHRCSCALGFGGRDCRE |
| | optional | | RADPCAARPCAHGGRCYAHFSGLVCACAPGYMGARCEFPVHPDGASA |
| | signal | | 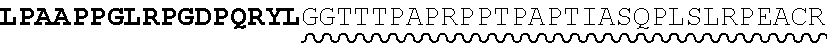 |
| | peptide, | | 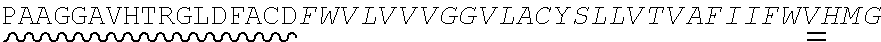 |
| | bold = DLL3 | | GGSGGGSAYPYDVPDYAAYPYDVPDYA |
| | region, | | |
| | italics = | | |
| | transmembrane | | |
| | helix; | | |
| | wavy | | |
| | underline = | | |
| | hinge region; | | |
| | double | | |
| | underline = | | |
| | optional | | |
| | affinity | | |
| | tag) | | |
| 37 | CD8aSP- | Synthetic | MALPVTALLLPLALLLHAARPAGVFELQIHSFGPGPGPGAPRSPCS |
| | DLL3tr- | | ARLPCRLFFRVCLKPGLSEEAAESPCALGAALSARGPVYTEQPGAP |
| | CD8aH- | | APDLPLPDGLLQVPFRDAWPGTFSFIIETWREELGDQIGGPAWSLL |
| | CD28TM | | ARVAGRRRLAAGGPWARDIQRAGAWELRFSYRARCEPPAVGTACTR |
| | (NP247)- | | LCRPRSAPSRCGPGLRPCAPLEDECEAPLVCRAGCSPEHGFCEQPG |
| | DLL3 portion | | 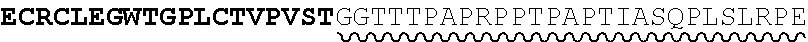 |
| | spans DLL3 | | 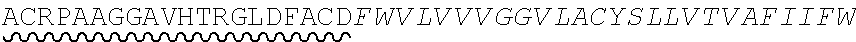 |
| | residues | | VHMGGGSGGGSAYPYDVPDYAAYPYDVPDYA |
| | 27-254 | | |
| | (underline = | | |
| | optional | | |
| | signal | | |
| | peptide, | | |
| | bold = DLL3 | | |
| | region, | | |
| | italics = | | |
| | transmembrane | | |
| | helix; | | |
| | wavy | | |
| | underline= | | |
| | hinge | | |
| | region; | | |
| | double | | |
| | underline = | | |
| | optional | | |
| | affinity | | |
| | tag) | | |
| 38 | GM(SP)- | Synthetic | MLLLVTSLLLCELPHPAFLLIPAGVFELQIHSFGPGPGPGAPRSPC |
| | DLL3tr- | | SARLPCRLFFRVCLKPGLSEEAAESPCALGAALSARGPVYTEQPGA |
| | huIgG4Fc- | | PAPDLPLPDGLLQVPFRDAWPGTFSFIIETWREELGDQIGGPAWSL |
| | CD4TM | | LARVAGRRRLAAGGPWARDIQRAGAWELRFSYRARCEPPAVGTACT |
| | (NP248)- | | RLCRPRSAPSRCGPGLRPCAPLEDECEAPLVCRAGCSPEHGFCEQP |
| | DLL3 | | 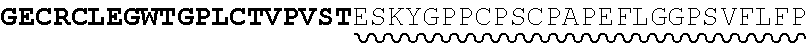 |
| | portion | | 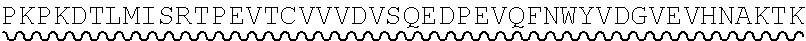 |
| | spans | | 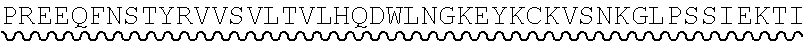 |
| | DLL3 | | 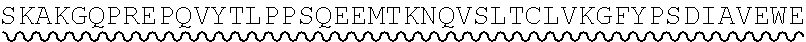 |
| | residues | | 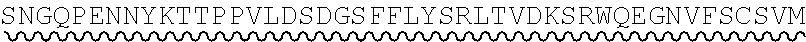 |
| | 27-254 | | 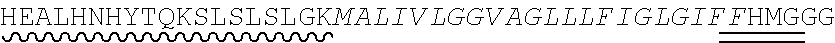 |
| | (underline = | | SGGGSAYPYDVPDYAAYPYDVPDYA |
| | optional | | |
| | signal | | |
| | peptide, | | |
| | bold = DLL3 | | |
| | region, | | |
| | italics = | | |
| | transmembrane | | |
| | helix; | | |
| | wavy | | |
| | underline = | | |
| | hinge | | |
| | region; | | |
| | double | | |
| | underline = | | |
| | optional | | |
| | affinity | | |
| | tag | | |
| 39 | DLL3vCD | Synthetic | MCPRAARAPATLLLALGAVLWPAAGAAGVFELQIHSFGPGPGPGAPR |
| | 73 | | SPCSARLPCRLFFRVCLKPGLSEEAAESPCALGAALSARGPVYTEQP |
| | (NP249)- | | GAPAPDLPLPDGLLQVPFRDAWPGTFSFIIETWREELGDQIGGPAWS |
| | DLL3 | | LLARVAGRRRLAAGGPWARDIQRAGAWELRFSYRARCEPPAVGTACT |
| | portion | | RLCRPRSAPSRCGPGLRPCAPLEDECEAPLVCRAGCSPEHGFCEQPG |
| | spans | | ECRCLEGWTGPLCTVPVSTSSCLSPRGPSSATTGCLVPGPGPCDGNP |
| | DLL3 | | CANGGSCSETPRSFECTCPRGFYGLRCEVSGVTCADGPCFNGGLCVG |
| | residues | | GADPDSAYICHCPPGFQGSNCEKRVDRCSLQPCRNGGLCLDLGHALR |
| | 27-492 | | CRCRAGFAGPRCEHDLDDCAGRACANGGTCVEGGGAHRCSCALGFGG |
| | (underline = | | RDCRERADPCAARPCAHGGRCYAHFSGLVCACAPGYMGARCEFPVHP |
| | optional | | DGASALPAAPPGLRPGDPQRYLGGSSTGSHCHGSFSLIFLSLWAVIF |
| | signal | | VLYQ |
| | peptide, | | |
| | bold = DLL3 | | |
| | region, | | |
| | italics = | | |
| | transmembrane) | | |
| 40 | CD8aSP- | Synthetic | MALPVTALLLPLALLLHAARPEPPAVGTACTRLCRPRSAPSRCGPGL |
| | DLL3(DS | | RPCAPLEDECEAPLVCRAGCSPEHGFCEQPGECRCLEGWTGPLCTVP |
| | L,E1)- | | 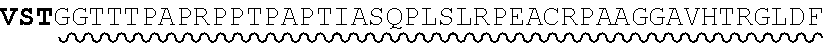 |
| | CD8aH- | | 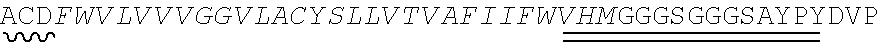 |
| | CD28TM | | DYAAYPYDVPDYA |
| | (NP296)- | | |
| | DLL3 portion | | |
| | spans DLL3 | | |
| | residues | | |
| | 179-254 | | |
| | (underline = | | |
| | optional | | |
| | signal | | |
| | peptide, | | |
| | bold = DLL3 | | |
| | region, | | |
| | italics = | | |
| | transmembrane | | |
| | helix; | | |
| | wavy | | |
| | underline = | | |
| | hinge | | |
| | region; | | |
| | double | | |
| | underline = | | |
| | optional | | |
| | affinity | | |
| | tag) | | |
| 41 | CD8aSP- | Synthetic | MALPVTALLLPLALLLHAARPEPPAVGTACTRLCRPRSAPSRCGPGL |
| | 2xDLL3 | | RPCAPLEDECEAPLVCRAGCSPEHGFCEQPGECRCLEGWTGPLCTVP |
| | (DSL,E1)- | | VSTGGEPPAVGTACTRLCRPRSAPSRCGPGLRPCAPLEDECEAPLVC |
| | CD8aH- | | 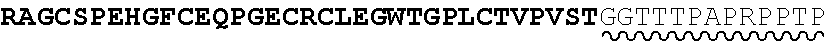 |
| | CD28TM | | 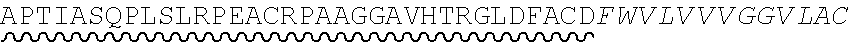 |
| | (NP298)- | | YSLLVTVAFIIFWVHMGGGSGGGSAYPYDVPDYAAYPYDVPDYA |
| | DLL3 | | |
| | portion | | |
| | spans | | |
| | DLL3 | | |
| | residues | | |
| | 179-254 | | |
| | (underline = | | |
| | optional | | |
| | signal | | |
| | peptide, | | |
| | bold = DLL3 | | |
| | region, | | |
| | italics = | | |
| | transmembrane | | |
| | helix; | | |
| | wavy | | |
| | underline = | | |
| | hinge | | |
| | region; | | |
| | double | | |
| | underline = | | |
| | optional | | |
| | affinity | | |
| | tag) | | |
| 42 | CD8aSP- | Synthetic | MALPVTALLLPLALLLHAARPAGVFELQIHSFGPGPGPGAPRSPCSA |
| | DLL3(C2 | | 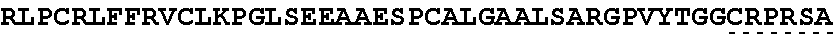 |
| | mut+DSL_E1)- | | 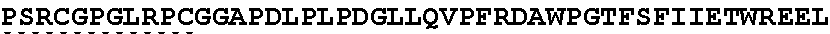 |
| | CD28TM | | 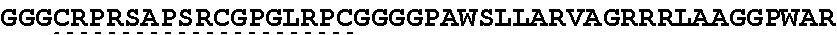 |
| | (NP299)- | | DIQRAGAWELRFSYRARCEPPAVGTACTRLCRPRSAPSRCGPGLRPC |
| | DLL3 | | APLEDECEAPLVCRAGCSPEHGFCEQPGECRCLEGWTGPLCTVPVST |
| | portion | | 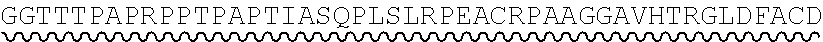 |
| | spans | | FWVLVVVGGVLACYSLLVTVAFIIFWVHMGGGSGGGSAYPYDVPDYA |
| | DLL3 | | AYPYDVPDYA |
| | residues | | |
| | 27-254 | | |
| | (underline = | | |
| | optional | | |
| | signal | | |
| | peptide, | | |
| | bold = DLL3 | | |
| | region, | | |
| | dashed | | |
| | underline = | | |
| | multiple | | |
| | copies of | | |
| | DLL3 | | |
| | epitope, | | |
| | italics = | | |
| | transmembrane | | |
| | helix; | | |
| | wavy | | |
| | underline = | | |
| | hinge | | |
| | region; | | |
| | double | | |
| | underline = | | |
| | optional | | |
| | affinity | | |
| | tag) | | |
| 43 | CD8aSP- | Synthetic | 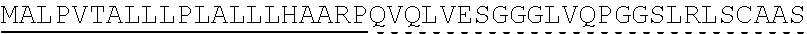 |
| | Nb- | | 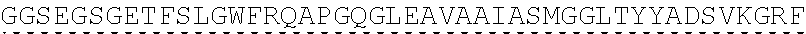 |
| | DLL3(DS | | 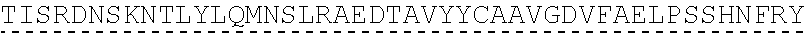 |
| | L,E1)- | | 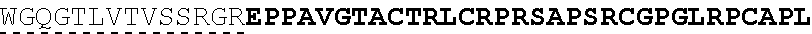 |
| | CD8aH- | | 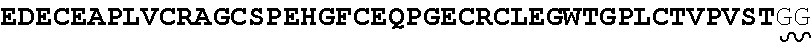 |
| | CD28TM | | 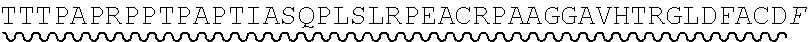 |
| | (NP302)- | | WVLVVVGGVLACYSLLVTVAFIIFWVHMGGGSGGGSAYPYDVPDYA |
| | DLL3 | | AYPYDVPDYA |
| | portion | | |
| | spans | | |
| | DLL3 | | |
| | residues | | |
| | 179-254 | | |
| | (underline = | | |
| | optional | | |
| | signal | | |
| | peptide, | | |
| | bold = DLL3 | | |
| | region, | | |
| | dotted | | |
| | underline = | | |
| | nanobody | | |
| | sequence, | | |
| | italics = | | |
| | transmembrane | | |
| | helix; | | |
| | wavy | | |
| | underline = | | |
| | hinge | | |
| | region; | | |
| | double | | |
| | underline= | | |
| | optional | | |
| | affinity | | |
| | tag) | | |
| 44 | CD8aSP- | Synthetic | 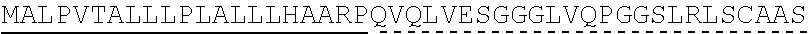 |
| | Nb(2xDL | | 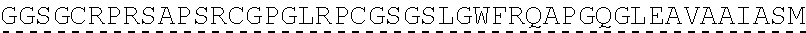 |
| | L3epi)- | | 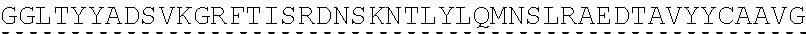 |
| | DLL3(DS | | 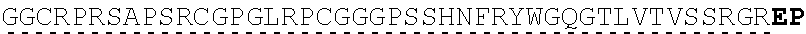 |
| | L,E1)- | | PAVGTACTRLCRPRSAPSRCGPGLRPCAPLEDECEAPLVCRAGCSP |
| | CD8aH- | | 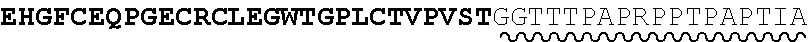 |
| | CD28TM | | 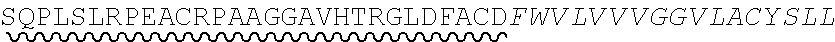 |
| | (NP303)- | | VTVAFIIFWVHMGGGSGGGSAYPYDVPDYAAYPYDVPDYA |
| | DLL3 | | |
| | portion | | |
| | spans | | |
| | DLL3 | | |
| | residues | | |
| | 179-254 | | |
| | (underline = | | |
| | optional | | |
| | signal | | |
| | peptide, | | |
| | bold = DLL3 | | |
| | region, | | |
| | dotted | | |
| | underline = | | |
| | nanobody | | |
| | sequence, | | |
| | italics = | | |
| | transmembrane | | |
| | helix; | | |
| | wavy | | |
| | underline = | | |
| | hinge | | |
| | region; | | |
| | double | | |
| | underline = | | |
| | optional | | |
| | affinity | | |
| | tag) | | |
| 94 | CD8aSP- | Synthetic | 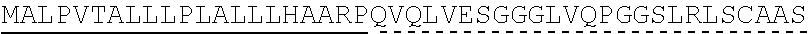 |
| | Nb(2xDL | | 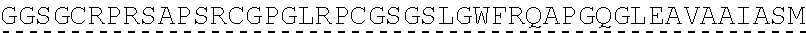 |
| | L3epi)- | | 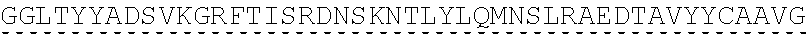 |
| | CD8aH- | | 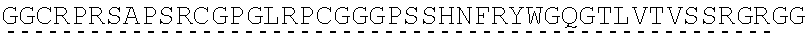 |
| | CD28TM | | 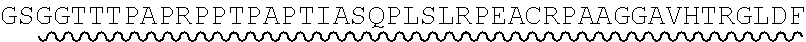 |
| | (NP304) | | 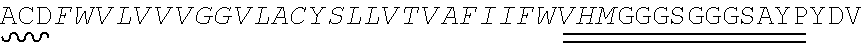 |
| | (underline = | | PDYAAYPYDVPDYA |
| | optional | | |
| | signal | | |
| | peptide, | | |
| | bold = DLL3 | | |
| | region, | | |
| | dotted | | |
| | underline = | | |
| | nanobody | | |
| | sequence, | | |
| | italics = | | |
| | transmembrane | | |
| | helix; | | |
| | wavy | | |
| | underline = | | |
| | hinge | | |
| | region; | | |
| | double | | |
| | underline = | | |
| | optional | | |
| | affinity | | |
| | tag) | | |
| 94 | GM(SP)- | Synthetic | AGVFELQIHSFGPGPGPGAPRSPCSARLPCRLFFRVCLKPGLSEEA |
| | DLL3tr- | | AESPCALGAALSARGPVYTEQPGAPAPDLPLPDGLLQVPFRDAWPG |
| | huIgG4Fc- | | TFSFIIETWREELGDQIGGPAWSLLARVAGRRRLAAGGPWARDIOR |
| | CD4TM | | AGAWELRFSYRARCEPPAVGTACTRLCRPRSAPSRCGPGLRPCAPL |
| | (NP248)- | | 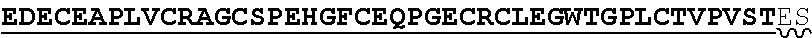 |
| | DLL3 | | 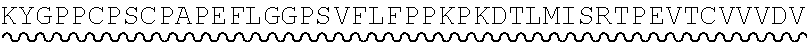 |
| | portion | | 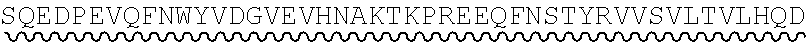 |
| | spans | | 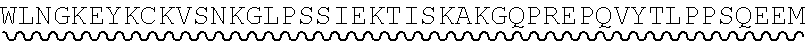 |
| | DLL3 | | 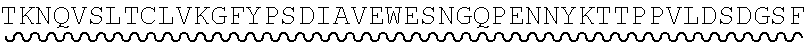 |
| | residues | | 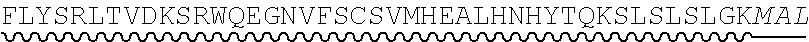 |
| | 27-254 | | IVLGGVAGLLLFIGLGIFF |
| | (bold = | | |
| | DLL3 region, | | |
| | italics = | | |
| | transmembrane | | |
| | helix; | | |
| | wavy | | |
| | underline = | | |
| | hinge | | |
| | region) | | |
| | Minimal | | |
| | sequence | | |
| 46 | DLL3vCD | Synthetic | AGVFELQIHSFGPGPGPGAPRSPCSARLPCRLFFRVCLKPGLSEEA |
| | 73 | | AESPCALGAALSARGPVYTEOPGAPAPDLPLPDGLLOVPFRDAWPG |
| | (NP249)- | | TFSFIIETWREELGDQIGGPAWSLLARVAGRRRLAAGGPWARDIOR |
| | DLL3 | | AGAWELRFSYRARCEPPAVGTACTRLCRPRSAPSRCGPGLRPCAPL |
| | portion | | EDECEAPLVCRAGCSPEHGFCEOPGECRCLEGWTGPLCTVPVSTSS |
| | spans | | CLSPRGPSSATTGCLVPGPGPCDGNPCANGGSCSETPRSFECTCPR |
| | DLL3 | | GFYGLRCEVSGVTCADGPCFNGGLCVGGADPDSAYICHCPPGFOGS |
| | residues | | NCEKRVDRCSLQPCRNGGLCLDLGHALRCRCRAGFAGPRCEHDLDD |
| | 27-492 | | CAGRACANGGTCVEGGGAHRCSCALGFGGRDCRERADPCAARPCAH |
| | (bold = | | GGRCYAHFSGLVCACAPGYMGARCEFPVHPDGASALPAAPPGLRPG |
| | DLL3 region, | | DPORYLGGSSTGSHCHGSFSLIFLSLWAVIFVLYQ |
| | italics = | | |
| | transmembrane | | |
| | helix; | | |
| | wavy | | |
| | underline = | | |
| | hinge | | |
| | region) | | |
| | minimal | | |
| | sequence | | |
| 47 | CD8aSP- | SynthetiC | EPPAVGTACTRLCRPRSAPSRCGPGLRPCAPLEDECEAPLVCRAGC |
| | DLL3(DS | | 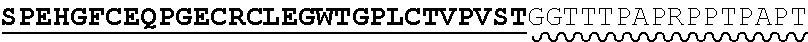 |
| | L,E1)- | | 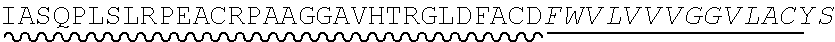 |
| | CD8aH- | | LLVTVAFIIFWVHM |
| | CD28TM | | |
| | (NP296)- | | |
| | DLL3 | | |
| | portion | | |
| | spans | | |
| | DLL3 | | |
| | residues | | |
| | 179-254 | | |
| | (bold = | | |
| | DLL3 region, | | |
| | italics = | | |
| | transmembrane | | |
| | helix; | | |
| | wavy | | |
| | underline = | | |
| | hinge | | |
| | region) | | |
| | minimal | | |
| | sequence | | |
| 48 | CD8aSP- | Synthetic | EPPAVGTACTRLCRPRSAPSRCGPGLRPCAPLEDECEAPLVCRAGC |
| | 2xDLL3 | | SPEHGFCEOPGECRCLEGWTGPLCTVPVSTGGEPPAVGTACTRLCR |
| | (DSL,E1)- | | PRSAPSRCGPGLRPCAPLEDECEAPLVCRAGCSPEHGFCEOPGECR |
| | CD8aH- | | 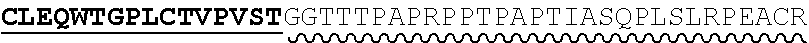 |
| | CD28TM | | 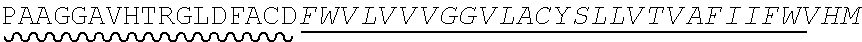 |
| | (NP298)- | | |
| | DLL3 | | |
| | portion | | |
| | spans | | |
| | DLL3 | | |
| | residues | | |
| | 179-254 | | |
| | (bold = | | |
| | DLL3 region, | | |
| | italics = | | |
| | transmembrane | | |
| | helix; | | |
| | wavy | | |
| | underline = | | |
| | hinge | | |
| | region) | | |
| 49 | CD8aSP- | Synthetic | AGVFELQIHSFGPGPGPGAPRSPCSARLPCRLFFRVCLKPGLSEEA |
| | DLL3(C2 | | 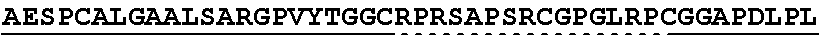 |
| | mut+DSL | | 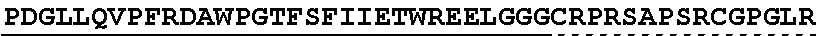 |
| | _E1)- | | 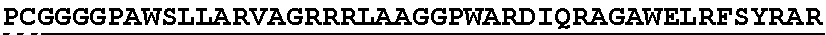 |
| | CD28TM | | CEPPAVGTACTRLCRPRSAPSRCGPGLRPCAPLEDECEAPLVCRAG |
| | (NP299)- | | 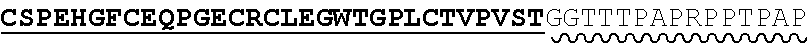 |
| | DLL3 | | 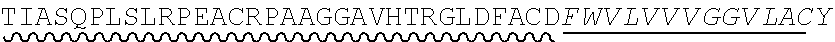 |
| | portion | | SLLVTVAFIIFWVHM |
| | spans | | |
| | DLL3 | | |
| | residues | | |
| | 27-254 | | |
| | (bold = DL | | |
| | L3 region, | | |
| | dashed | | |
| | underline = | | |
| | multiple | | |
| | copies of | | |
| | DLL3 | | |
| | epitope, | | |
| | italics = | | |
| | transmembrane | | |
| | helix; | | |
| | wavy | | |
| | underline = | | |
| | hinge | | |
| | region) | | |
| 50 | CD8aSP- | Synthetic | 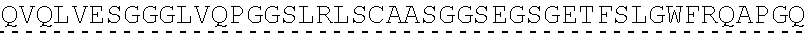 |
| | Nb- | | 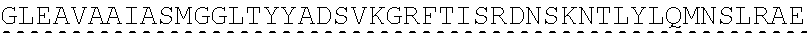 |
| | DLL3(DS | | 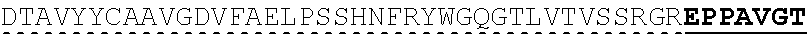 |
| | L,E1)- | | ACTRLCRPRSAPSRCGPGLRPCAPLEDECEAPLVCRAGCSPEHGFC |
| | CD8aH- | | 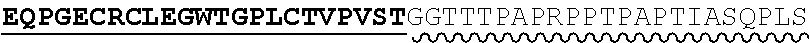 |
| | CD28TM | | 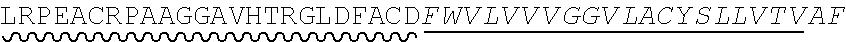 |
| | (NP302)- | | IIFWVHM |
| | DLL3 | | |
| | portion | | |
| | spans | | |
| | DLL3 | | |
| | residues | | |
| | 179-254 | | |
| | (bold = DL | | |
| | L3 region, | | |
| | dotted | | |
| | underline = | | |
| | nanobody | | |
| | sequence, | | |
| | italics = | | |
| | transmembrane | | |
| | helix; | | |
| | wavy | | |
| | underline = | | |
| | hinge | | |
| | region) | | |
| | minimal | | |
| | sequence | | |
| 51 | CD8aSP- | Synthetic | 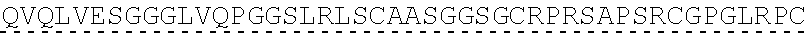 |
| | Nb(2xDL | | 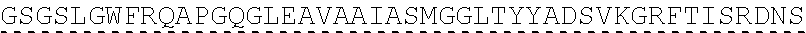 |
| | L3epi)- | | 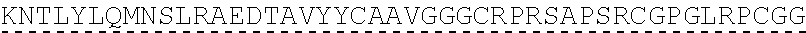 |
| | DLL3(DS | | 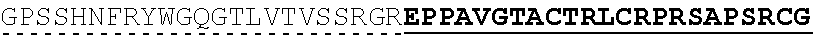 |
| | L,E1)- | | PGLRPCAPLEDECEAPLVCRAGCSPEHGFCEOPGECRCLEGWTGPL |
| | CD8aH- | | 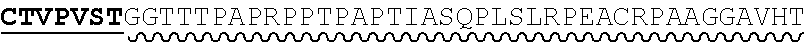 |
| | CD28TM | | 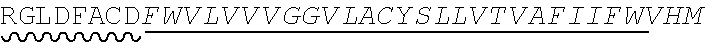 |
| | (NP303)- | | |
| | DLL3 | | |
| | portion | | |
| | spans | | |
| | DLL3 | | |
| | residues | | |
| | 179-254 | | |
| | (bold = DL | | |
| | L3 region, | | |
| | dotted | | |
| | underline = | | |
| | nanobody | | |
| | sequence, | | |
| | italics = | | |
| | transmembrane | | |
| | helix; | | |
| | wavy | | |
| | underline = | | |
| | hinge | | |
| | region) | | |
| | minimal | | |
| | sequence | | |
| 52 | CD8aSP- | Synthetic | 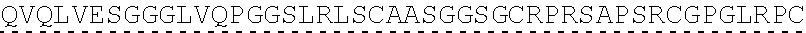 |
| | Nb(2xDL | | 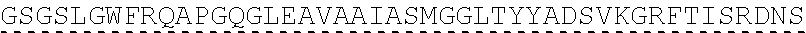 |
| | L3epi)- | | 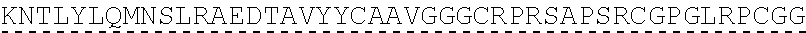 |
| | CD8aH- | | 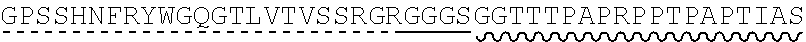 |
| | CD28TM | | 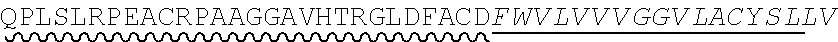 |
| | (NP304) | | TVAFIIFWVHM |
| | (bold = DL | | |
| | L3 region, | | |
| | dotted | | |
| | underline = | | |
| | nanobody | | |
| | sequence, | | |
| | italics = | | |
| | transmembrane | | |
| | helix; | | |
| | wavy | | |
| | underline = | | |
| | hinge | | |
| | region) | | |
| 53 | Human | Human | MVSPRMSGLLSQTVILALIFLPQTRPAGVFELQIHSFGPGPGPGAP |
| | DLL3 full | | RSPCSARLPCRLFFRVCLKPGLSEEAAESPCALGAALSARGPVYTE |
| | length | | QPGAPAPDLPLPDGLLQVPFRDAWPGTFSFIIETWREELGDQIGGP |
| | sequence | | AWSLLARVAGRRRLAAGGPWARDIQRAGAWELRFSYRARCEPPAVG |
| | | | TACTRLCRPRSAPSRCGPGLRPCAPLEDECEAPLVCRAGCSPEHGF |
| | | | CEQPGECRCLEGWTGPLCTVPVSTSSCLSPRGPSSATTGCLVPGPG |
| | | | PCDGNPCANGGSCSETPRSFECTCPRGFYGLRCEVSGVTCADGPCF |
| | | | NGGLCVGGADPDSAYICHCPPGFQGSNCEKRVDRCSLQPCRNGGLC |
| | | | LDLGHALRCRCRAGFAGPRCEHDLDDCAGRACANGGTCVEGGGAHR |
| | | | CSCALGFGGRDCRERADPCAARPCAHGGRCYAHFSGLVCACAPGYM |
| | | | GARCEFPVHPDGASALPAAPPGLRPGDPQRYLLPPALGLLVAAGVA |
| | | | GAALLLVHVRRRGHSQDAGSRLLAGTPEPSVHALPDALNNLRTQEG |
| | | | SGDGPSSSVDWNRPEDVDPQGIYVISAPSIYAREVATPLFPPLHTG |
| | | | RAGQRQHLLFPYPSSILSVK |
| 54 | Human | Human | MWNLLHETDSAVATARRPRWLCAGALVLAGGFFLLGFLFGWFIKSS |
| | PSMA full | | NEATNITPKHNMKAFLDELKAENIKKFLYNFTQIPHLAGTEQNFQL |
| | length | | AKQIQSQWKEFGLDSVELAHYDVLLSYPNKTHPNYISIINEDGNEI |
| | sequence | | FNTSLFEPPPPGYENVSDIVPPFSAFSPQGMPEGDLVYVNYARTED |
| | | | FFKLERDMKINCSGKIVIARYGKVFRGNKVKNAQLAGAKGVILYSD |
| | | | PADYFAPGVKSYPDGWNLPGGGVQRGNILNLNGAGDPLTPGYPANE |
| | | | YAYRRGIAEAVGLPSIPVHPIGYYDAQKLLEKMGGSAPPDSSWRGS |
| | | | LKVPYNVGPGFTGNFSTQKVKMHIHSTNEVTRIYNVIGTLRGAVEP |
| | | | DRYVILGGHRDSWVFGGIDPQSGAAVVHEIVRSFGTLKKEGWRPRR |
| | | | TILFASWDAEEFGLLGSTEWAEENSRLLQERGVAYINADSSIEGNY |
| | | | TLRVDCTPLMYSLVHNLTKELKSPDEGFEGKSLYESWTKKSPSPEF |
| | | | SGMPRISKLGSGNDFEVFFQRLGIASGRARYTKNWETNKFSGYPLY |
| | | | HSVYETYELVEKFYDPMFKYHLTVAQVRGGMVFELANSIVLPFDCR |
| | | | DYAVVLRKYADKIYSISMKHPQEMKTYSVSFDSLFSAVKNFTEIAS |
| | | | KFSERLQDFDKSNPIVLRMMNDQLMFLERAFIDPLGLPDRPFYRHV |
| | | | IYAPSSHNKYAGESFPGIYDALFDIESKVDPSKAWGEVKRQIYVAA |
| | | | FTVQAAAE |
| 55 | Human | Human | MDMADEPLNGSHTWLSIPFDLNGSVVSTNTSNQTEPYYDLTSNAVL |
| | SSTR2 | | TFIYFVVCIIGLCGNTLVIYVILRYAKMKTITNIYILNLAIADELF |
| | full length | | MLGLPFLAMQVALVHWPFGKAICRVVMTVDGINQFTSIFCLTVMSI |
| | sequence | | DRYLAVVHPIKSAKWRRPRTAKMITMAVWGVSLLVILPIMIYAGLR |
| | | | SNQWGRSSCTINWPGESGAWYTGFIIYTFILGFLVPLTIICLCYLF |
| | | | IIIKVKSSGIRVGSSKRKKSEKKVTRMVSIVVAVFIFCWLPFYIFN |
| | | | VSSVSMAISPTPALKGMFDFVVVLTYANSCANPILYAFLSDNFKKS |
| | | | FQNVLCLVKVSGTDDGERSDSKQDKSRLNETTETQRTLLNGDLQTS |
| | | | I |
| 56 | CD8a full | Human | MALPVTALLLPLALLLHAARPSQFRVSPLDRTWNLGETVELKCQVL |
| | length | | LSNPTSGCSWLFQPRGAAASPTFLLYLSQNKPKAAEGLDTQRFSGK |
| | human | | RLGDTFVLTLSDFRRENEGYYFCSALSNSIMYFSHFVPVFLPAKPT |
| | sequence | | TTPAPRPPTPAPTIASQPLSLRPEACRPAAGGAVHTRGLDFACDIY |
| | | | IWAPLAGTCGVLLLSLVITLYCNHRNRRRVCKCPRPVVKSGDKPSL |
| | | | SARYV |
| 57 | CD8b full | Human | MRPRLWLLLAAQLTVLHGNSVLQQTPAYIKVQTNKMVMLSCEAKIS |
| | length | | LSNMRIYWLRQRQAPSSDSHHEFLALWDSAKGTIHGEEVEQEKIAV |
| | human | | FRDASRFILNLTSVKPEDSGIYFCMIVGSPELTFGKGTQLSVVDFL |
| | sequence | | PTTAQPTKKSTLKKRVCRLPRPETQKGPLCSPITLGLLVAGVLVLL |
| | | | VSLGVAIHLCCRRRRARLRFMKQFYK |
| 58 | Human | Human | ASTKGPSVFPLAPCSRSTSESTAALGCLVKDYFPEPVTVSWNSGAL |
| | IgG4 | | TSGVHTFPAVLQSSGLYSLSSVVTVPSSSLGTKTYTCNVDHKPSNT |
| | heavy | | KVDKRVESKYGPPCPSCPAPEFLGGPSVFLFPPKPKDTLMISRTPE |
| | chain full | | VTCVVVDVSQEDPEVQFNWYVDGVEVHNAKTKPREEQFNSTYRVVS |
| | length | | VLTVLHQDWLNGKEYKCKVSNKGLPSSIEKTISKAKGQPREPQVYT |
| | sequence | | LPPSQEEMTKNQVSLTCLVKGFYPSDIAVEWESNGQPENNYKTTPP |
| | | | VLDSDGSFFLYSRLTVDKSRWQEGNVFSCSVMHEALHNHYTQKSLS |
| | | | LSLGK |
| 59 | Human | Human | TCPPCPAPELLGGPSVFLFPPKPKDTLMISRTPEVTCVVVDVSHED |
| | IgG1 | | PEVKFNWYVDGVEVHNAKTKPREEQYNSTYRVVSVLTVLHQDWLNG |
| | heavy | | KEYKCKVSNKALPAPIEKTISKAKGQPREPQVYTLPPSRDELTKNQ |
| | chain full | | VSLTCLVKGFYPSDIAVEWESNGQPENNYKTTPPVLDSDGSFFLYS |
| | length | | KLTVDKSRWQQGNVFSCSVMHEALHNHYTQKSLSLSPGK |
| | sequence | | |
| 60 | Human | Human | VECPPCPAPPVAGPSVFLFPPKPKDTLMISRTPEVTCVVVDVSHEDP |
| | IgG2 | | EVQFNWYVDGVEVHNAKTKPREEQFNSTFRVVSVLTVVHQDWLNGKE |
| | heavy | | YKCKVSNKGLPAPIEKTISKTKGQPREPQVYTLPPSREEMTKNQVSL |
| | chain full | | TCLVKGFYPSDISVEWESNGQPENNYKTTPPMLDSDGSFFLYSKLTV |
| | length | | DKSRWQQGNVFSCSVMHEALHNHYTQKSLSLSPGK |
| | sequence | | |
| 61 | Human | Human | TPPPCPRCPAPELLGGPSVFLFPPKPKDTLMISRTPEVTCVVVDVS |
| | IgG3 | | HEDPEVQFKWYVDGVEVHNAKTKPREEQYNSTFRVVSVLTVLHQDW |
| | heavy | | LNGKEYKCKVSNKALPAPIEKTISKTKGQPREPQVYTLPPSREEMT |
| | chain full | | KNQVSLTCLVKGFYPSDIAVEWESSGQPENNYNTTPPMLDSDGSFF |
| | length | | LYSKLTVDKSRWQQGNIFSCSVMHEALHNRFTQKSLSLSPGK |
| | sequence | | |
| 62 | Human | Human | ESKYGPPCPSCPAPEFLGGPSVFLFPPKPKDTLMISRTPEVTCVVV |
| | IgG4 | | DVSQEDPEVQFNWYVDGVEVHNAKTKPREEQFNSTYRVVSVLTVLH |
| | heavy | | QDWLNGKEYKCKVSNKGLPSSIEKTISKAKGQPREPQVYTLPPSQE |
| | chain full | | EMTKNQVSLTCLVKGFYPSDIAVEWESNGQPENNYKTTPPVLDSDG |
| | length | | SFFLYSRLTVDKSRWQEGNVFSCSVMHEALHNHYTQKSLSLSLGK |
| | sequence | | |
| 63 | Deactivated | Human | QVQLVESGGGLVQPGGSLRLSCAASGGSEGSGETFSLGWFRQAPGQ |
| | VHH | | GLEAVAAIASMGGLTYYADSVKGRFTISRDNSKNTLYLQMNSLRAE |
| | nanobody | | DTAVYYCAAVGDVFAELPSSHNFRYWGQGTLVTVSSRGR |
| | sequence | | |
| 64 | DLL3 | Human | EPPAVGTACTRLCRPRSAPSRCGPGLRPCAPLEDECEAPLVCRAGC |
| | portion | | SPEHGFCEQPGECRCLEGWTGPLCTVPVST |
| | 179-254 | | |
| 65 | DLL3 | Human | CRPRSAPSRCGPGLRPC |
| | minimal | | |
| | epitope | | |
| 66 | CD8a | Human | GGTTTPAPRPPTPAPTIASQPLSLRPEACRPAAGGAVHTRGLDFAC |
| | hinge | | D |
| | region | | |
| | (CD8a | | |
| | residues | | |
| | 138-182) | | |
| 67 | IgG4 | Human | ESKYGPPCPSCPAPEFLGGPSVFLFPPKPKDTLMISRTPEVTCVVV |
| | hinge | | DVSQEDPEVQFNWYVDGVEVHNAKTKPREEQFNSTYRVVSVLTVLH |
| | region | | QDWLNGKEYKCKVSNKGLPSSIEKTISKAKGQPREPQVYTLPPSQE |
| | (IgG4 | | EMTKNQVSLTCLVKGFYPSDIAVEWESNGQPENNYKTTPPVLDSDG |
| | heavy | | SFFLYSRLTVDKSRWQEGNVFSCSVMHEALHNHYTQKSLSLSLGK |
| | chain | | |
| | residues | | |
| | 219-238) | | |
| 68 | DLL3 | Human | LPPALGLLVAAGVAGAALLLVHVRRRGHS |
| | transmembrane | | |
| | domain | | |
| 69 | PSMA | Human | WLCAGALVLAGGFFLLGFLFGWFI |
| | transmembrane | | |
| | domain | | |
| 70 | SSTR2 | Human | MDMADEPLNGSHTWLSIPFDLNGSVVSTNTSNQTEPYYDLTSNAVL |
| | (contains | | TFIYFVVCIIGLCGNTLVIYVILRYAKMKTITNIYILNLAIADELF |
| | transmembrane | | MLGLPFLAMQVALVHWPFGKAICRVVMTVDGINQFTSIFCLTVMSI |
| | domain) | | DRYLAVVHPIKSAKWRRPRTAKMITMAVWGVSLLVILPIMIYAGLR |
| | | | SNOWGRSSCTINWPGESGAWYTGFIIYTFILGFLVPLTIICLCYLF |
| | | | IIIKVKSSGIRVGSSKRKKSEKKVTRMVSIVVAVFIFCWLPFYIFN |
| | | | VSSVSMAISPTPALKGMFDFVVVLTYANSCANPILYAFLSDNFKKS |
| | | | FQNVLCLVKVSGTDDGERSDSKQDKSRLNETTETQRTLLNGDLQTS |
| | | | I |
| 71 | PD-L1 | Human | THLVILGAILLCLGVALTFIF |
| | transmembrane | | |
| | domain | | |
| 72 | EGFR | Human | IATGMVGALLLLLVVALGIGLFM |
| | transmembrane | | |
| | domain | | |
| 73 | CD28 | Human | FWVLVVVGGVLACYSLLVTVAFIIFWV |
| | transmembrane | | |
| | domain | | |
| 74 | CD4 | Human | MALIVLGGVAGLLLFIGLGIFF |
| | transmembrane | | |
| | domain | | |
| 75 | ICOS | Human | FWLPIGCAAFVVVCILGCILI |
| | transmembrane | | |
| | domain | | |
| 76 | CD73 GPI | Human | STGSHCHGSFSLIFLSLWAVIFVLYQ |
| | anchor | | |
| | region | | |
| 77 | C1 domain | Human | GSSGSSGTTLASIGKDIIEDADGIAMPHOWLEGNLPVSAKCTVCDKT |
| | | | CGSVLRLQDWRCLWCKAMVHTSCKESLLTKCSGPSSG |
| 78 | C2 domain | Human | AGVFELQIHSFGPGPGPGAPRSPCSARLPCRLFFRVCLKPGLSEEAA |
| | | | ESPCALGAALSARGPVYTEQPGAPAPDLPLPDGLLQVPFRDAWPGTF |
| | | | SFIIETWREELGDQIGGPAWSLLARVAGRRRLAAGGPWARDIQRAGA |
| | | | WELRFSYR |
| 79 | PH | Human | GSSGSSGYGSEKKGYLLKKSDGIRKVWQRRKCSVKNGILTISHATSN |
| | domain | | RQPAKLNLLTCQVKPNAEDKKSFDLISHNRTYHFQAEDEQDYVAWIS |
| | | | VLTNSKEEALTMAFSGPSSG |
| 80 | FYVE | | SMEGLVLGQKQPTWVPDSEAPNCMNCQVKFTFTKRRHHCRACGKVFC |
| | domain | | GVCCNRKCKLQYLEKEARVCVVCYETISKAQAFERMMSPTGSN |
| 81 | ENTH | Human | GPLGSMAAAPPLRDRLSFLHRLPILLKGTSDDDVPCPGYLFEEIAKI |
| | domain | | SHESPGSSQCLLEYLLSRLHSSSGHGKLKVLKILLYLCSHGSSFFLL |
| | | | ILKRNSAFIQEAAAFAGPPDPLHGNSLYQKVRAAAQDLGSTLFSDTV |
| | | | LPLA |
| 82 | N/A | N/A | N/A |
| 83 | CD8b | Human | LPTTAQPTKKSTPKRRVCRLPRPETQKGPLCSP |
| | hinge | | |
| | region | | |
| 84 | CD8a | Human | IYIWAPLAGTCGVLLLSLVIT |
| | transmembrane | | |
| | domain | | |
| 85 | CD8b | Human | ITLGLLVAGVLVLLVSLGVAI |
| | transmembrane | | |
| | domain | | |
| 86 | CD28 | Human | PPPYLDNEKSNGTIIHVKGKHLCPSPLFPGPSKP |
| | hinge | | |
| | region | | |
| 87 | CD4 hinge | Human | LNPEAGMWQCLLSDSGQVLLESNIKVLPTWSTPVQP |
| | region | | |

### EXAMPLES

### Example 1—Detection, Imaging, or Treatment of a Cancer Cell Using Cell-Surface Expressible Biomarkers Described Herein

A vector (e.g. a viral vector or nanoplasmid vector) comprising coding sequence for a cell-surface expressible biomarker as described herein comprising an epitope under the control of a cancer-specific promoter (e.g. a promoter of a gene overexpressed in a cancer cell relative to a normal cell) is administered to the subject suspected of having a cancer. Various cells may take up the vector, but expression of the biomarker is favored in cancerous cells due to the cancer-specific promoter. The accessible epitope can then be detected by antibodies, antibody fragments or derivatives, or protein ligands that bind the epitope. These antibodies, antibody fragments or derivatives, or protein ligands can be labeled with PET-active radionuclides such as 124I or 68Ga that enable detection and/or imaging of the cancer cells. In some cases, for therapeutic purposes (e.g. theranostic purposes) against the cancer, these antibodies, antibody fragments or derivatives, or protein ligands can be labeled with high energy radionuclide beta emitters (such as 177Lu, 67Cu, or 90Y) or alpha emitters (such as 225 Ac, 211 At, 227Th) to kill the tumors. Finally, these antibodies, antibody fragments or derivatives, or protein ligands can be conjugated to other molecules which may exert toxic effects on the tumor cells.

### Example 2—Detection, Imaging, or Treatment of a Cancer Cell Using Cell-Surface Expressible Post-Translationally Modified Biomarkers Described Herein

A vector (e.g. a viral vector or nanoplasmid vector) comprising coding sequence for a cell-surface expressible biomarker as described herein comprising an activatable epitope under the control of a cancer-specific promoter (e.g. a promoter of a gene overexpressed in a cancer cell relative to a normal cell) is administered to the subject suspected of having a cancer. Various cells may take up the vector, but expression of the biomarker is favored in cancerous cells due to the cancer-specific promoter. In the event the biomarker is expressed in non-cancerous cells, the absence of the proper activating microenvironment inside or outside of the normal cell leaves the activatable epitope occluded and unable to be detected. When the biomarker containing the activatable epitope is expressed in a tumor microenvironment or inside of a tumor cell, the activatable epitope is revealed on the surface of the cancer cell and in a context in which the activatable epitope is accessible to binding. The accessible activatable epitope can then be detected by antibodies, antibody fragments or derivatives, or protein ligands that bind the epitope. These antibodies, antibody fragments or derivatives, or protein ligands can be labeled with PET-active radionuclides such as ¹²⁴I or ⁶⁸Ga that enable detection and/or imaging of the cancer cells. For therapeutic purposes (e.g. theranostic) against the cancer, these antibodies, antibody fragments or derivatives, or protein ligands can be labeled with high energy radionuclide beta emitters such as ¹⁷⁷Lu, ⁶⁷Cu, or ⁹⁰Y or alpha emitters such as ²²⁵Ac, ²¹¹At, ²²⁷Th to kill the tumors. Finally, these antibodies, antibody fragments or derivatives, or protein ligands can be conjugated to other molecules which may exert toxic effects on the tumor cells.

### Example 3-Construction of a pH-Sensitive, Self-Propagating, Secreted Cell Marker

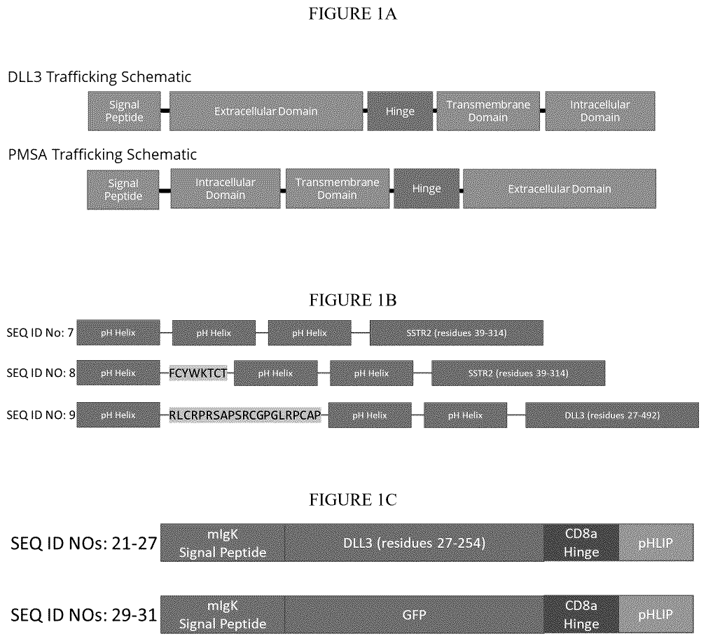

It was considered that cancer cells expressing the constructs shown in FIGS. 1-4 could also label neighboring cells and amplify the signal produced by delivery to a tumor if the construct was secretable and comprised a membrane-affinity domain such that it could be secreted and insert into the membrane of neighboring cells. It was additionally considered that such constructs could be pH-sensitive (and thus cancer microenvironment-sensitive) if the membrane affinity-domain comprised a pH-sensitive protein motif.

Accordingly, constructs of the type described in FIG. 5 were designed (SEQ ID NOs: 21-28) were designed, which comprise a cleavable signal peptide capable of driving secretion of the construct, a reporter domain linked to the signal peptide, and a pH-sensitive membrane affinity protein motif (pHLIP). It was considered that such constructs would be secreted by cancer cells, and only insert into neighboring cells within the cancer microenvironment since the membrane-affinity protein motif would only activate at low pH, transitioning from an unfolded, soluble state into a membrane-anchored helix (FIG. 6)

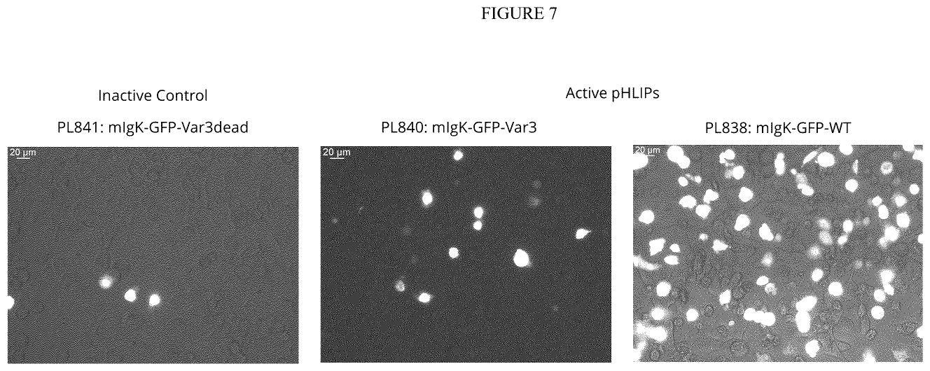

An initial question was whether constructs designed in this manner would propagate to nearby cells. Accordingly, an experiment (FIG. 7) was performed where untransfected cancer-derived H1299 cells were treated using media obtained from H1299 cells engineered to express comparable amounts of reporter construct comprising a cleavable mIgK secretion tag (SEQ ID NO: 32) fused to GFP and either a mutationally inactive membrane binding helix (mIgK-GFP-Var3dead) or active pH-sensitive membrane binding helices (mIgK-GFP-Var3 or mIgK-GFP-WT). As the H1299 cells themselves did not contain the expression construct, any GFP fluorescence was interpreted as coming from GFP-helices polypeptides that successfully inserted into the membrane of, or otherwise associated with, H1299 cells. The WT version of the pH-sensitive membrane-binding motif also appeared to show superior results to the Var3 version of the pH-sensitive membrane-binding motif.

The experiment media from cells expressing a construct with an active membrane-binding motif (mIgK-GFP-Var3 or mIgK-GFP-WT) induced increased numbers of fluorescent cells relative to cells containing the inactive membrane-binding motif (mIgK-GFP-Var3dead), indicating that the secretion tag-reporter-membrane binding domain construct was able to propagate to untransfected cells.

### Example 4—Designing and Engineering of DLL3 Constructs for Cell Surface Expression I

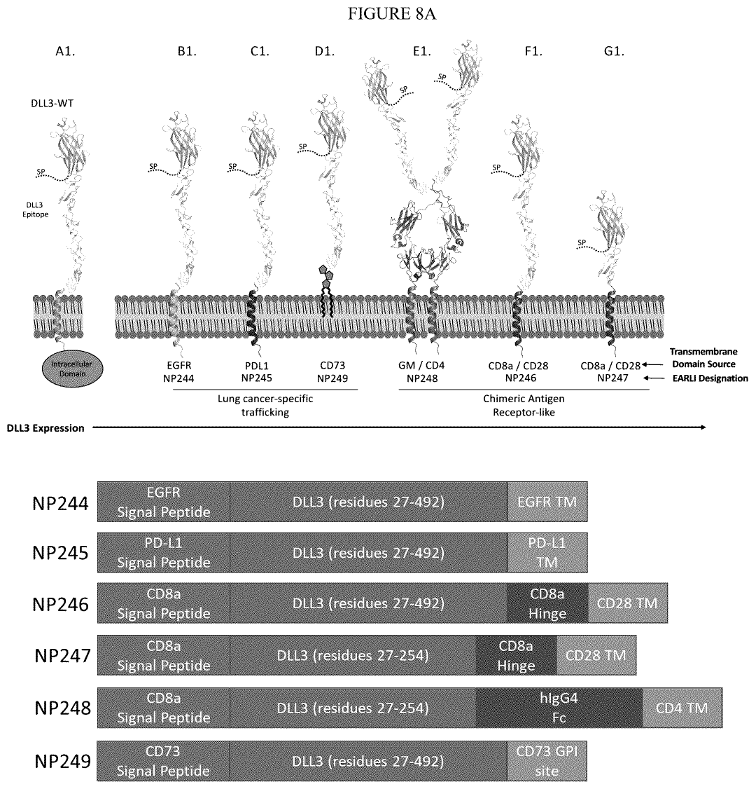

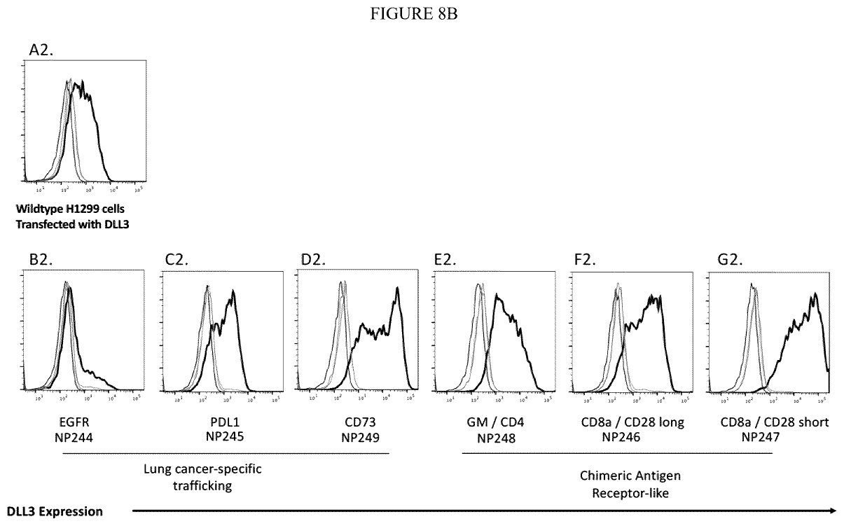

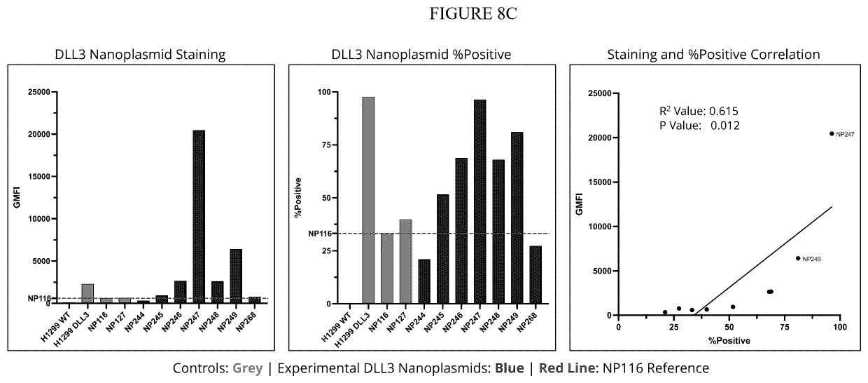

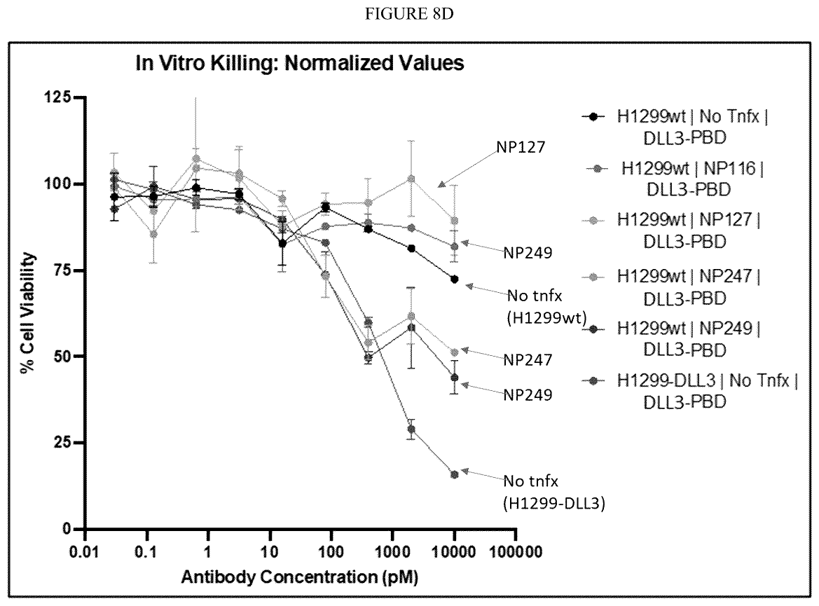

DLL3 is a membrane protein known to be located primarily in the Golgi or late endosome membranes and trafficked to the cell surface to a small degree when overexpressed in certain cancers (A1 in FIG. 8A). To engineer DLL3 with increased cell surface expression, two different approaches were used. The first approach utilized trafficking directives such as signal peptide (SP) and transmembrane/membrane-anchoring domains from other proteins known to reside on the cell surface of lung cancer cells. The SPs can direct trafficking of the extracellular domain across a membrane during translation and the transmembrane domain can anchor DLL3 to the cell membrane. Lung-specific trafficking directives can include EGFR (SP/transmembrane helix; NP244; B1 in FIG. 8A), PD-L1 (SP/transmembrane helix; NP245; C1 in FIG. 8A), and CD73 (SP/GPI-anchor motif; NP249; D1 in FIG. 8A). The second approach utilized different combinations of trafficking directives for cell surface trafficking of proteins on immune cells including GM-CSF SP/CD4 transmembrane helix (NP248; E1 in FIG. 8A) or CD8a SP/CD28 transmembrane helix (NP244 and NP247; F1 and G1, respectively, in FIG. 8A). To further improve expression and/or surface presentation, a shorter DLL3 sequence with less structural complexity was designed. For example, native signal peptide, transmembrane helix, and intracellular domain were removed from DLL3. NP244, NP245, NP249, NP248, and NP246 (A1-F1) constructs were designed to comprise amino acid residues 27-492 of DLL3. NP247 comprises a further truncated extracellular domain of DLL3, amino acid residues 27-254 (G1 in FIG. 8A). Amino acid sequences of NP244-NP249 are shown in Table 1.

Each of the six engineered DLL3 constructs (NP244-NP247) and two different wild-type DLL3 constructs (NP116 and NP127) was separately introduced to human non-small cell lung carcinoma, H1299 cells (also known as NCI-H1299 or CRL-5803) by transfection to analyze the expression of engineered DLL3. H1299 cells were then stained with fluorescently labeled anti-DLL3 antibodies and analyzed by fluorescence-activated cell sorting (FACS) to detect DLL3 expression. Panels A2-G2 in FIG. 8B, and NP116, NP127, NP244-NP249, and NP268 in FIG. 8C show the amount of DLL3 expressed on the cell surface corresponding to panels A1-G1 in FIG. 8A. While NP244 (FIGS. 8B-8C) did not increase the expression level of DLL3 on the cell surface, other constructs-NP245, NP249, NP248, NP246, and NP247-increased the expression level of DLL3 (FIGS. 8B-8C) compared to the wild-type DLL3 (A2 in FIG. 8B and NP116 and NP127 FIG. 8C). Specifically, NP249 and NP247 increased percent positive to ˜80% and ˜94%, respectively (FIG. 8C).

Next, in vitro cell killing assay was performed to determine the ability of anti-DLL3 antibodies conjugated with a cytotoxic payload (anti-DLL3-PBD) to induce cytotoxicity. The anti-DLL3-PBD was introduced to H1299 cells transfected with NP247 (G1 in FIG. 8A), NP249 (D1 in FIG. 8A), NP116 (wild-type control), or NP127 (wild-type control). Compared to cells expressing NP116 or NP127, cells expressing NP247 or NP249 exhibited substantial loss in viability when treated with anti-DLL3-PBD (FIG. 8D). The results suggest that cells expressing engineered DLL3 that has increased expression level on the cell surface (i.e., NP247 or NP249) may be more susceptible to cell death when treated with anti-DLL3-PBD compared to cells expressing wild-type DLL3 (i.e., NP116 or NP127).

### Example 5—Designing and Engineering of DLL3 Constructs for Cell Surface Expression II

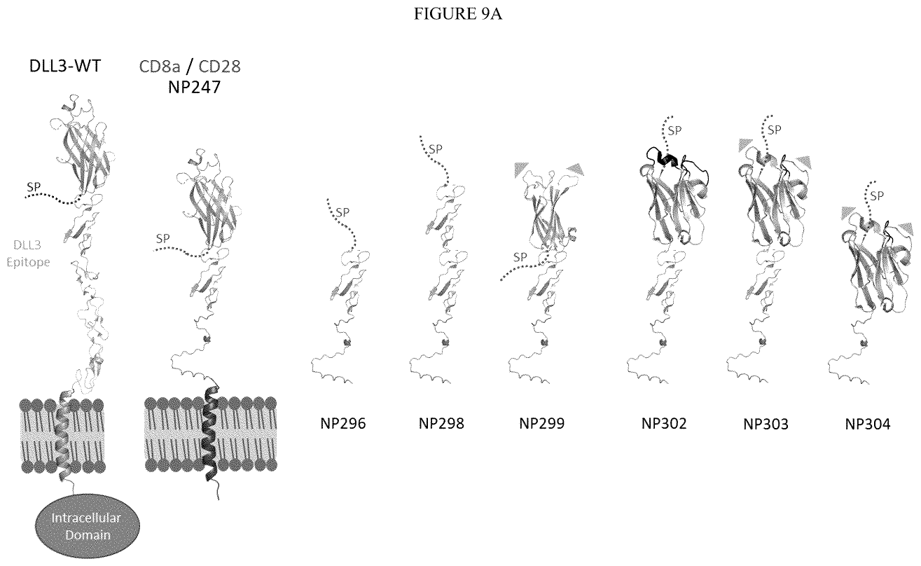

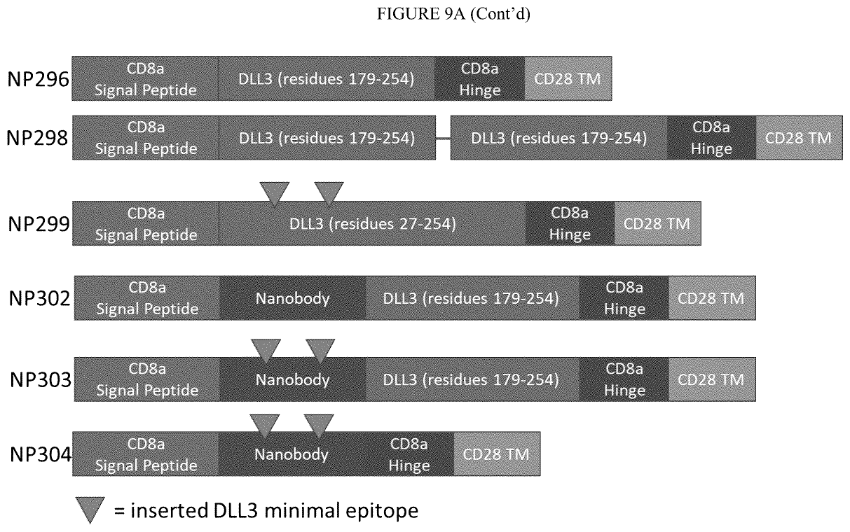

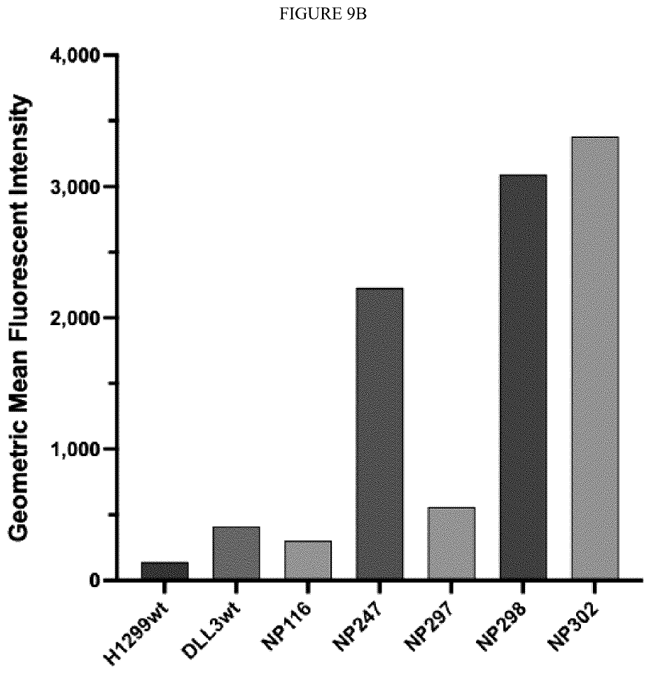

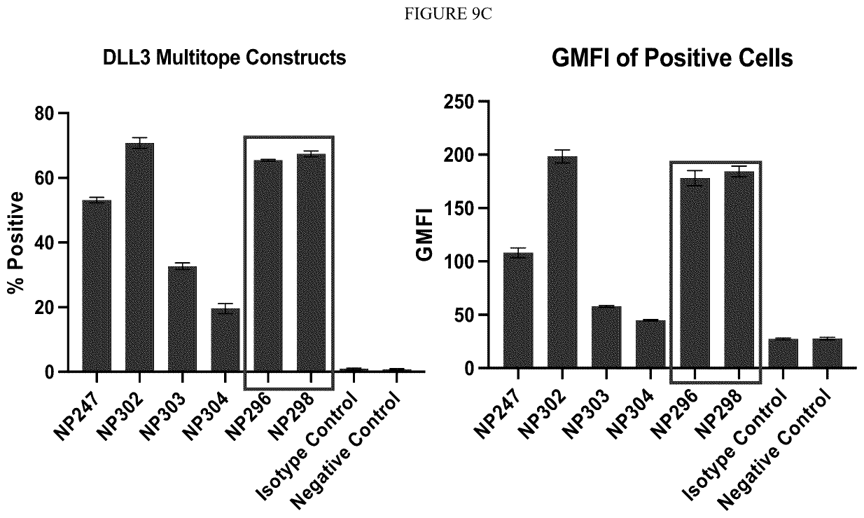

Six additional DLL3 constructs, NP296, NP298, NP299, NP302, NP303, and NP304, were designed based on NP247 (G1 in FIG. 8A) to identify a minimal DLL3 domain that is sufficient for interaction with the DLL3 antibody tracer (FIG. 9A). NP296 comprises amino acid residues 179-254 of DLL3. NP298 comprises amino acid residues 179-254 of DLL3 and a second DLL3 epitope, which was added with a goal of doubling the antibody binding capacity. NP299 comprises amino acid residues 27-254 of DLL3 and two DLL3 minimal epitopes inserted into the DLL3 C2 domain. NP302 comprises amino acid residues 179-254 of DLL3 and a soluble humanized nanobody structure that replaces a C2 domain of DLL3. NP303 comprises amino acid residues 179-254 of DLL3, a soluble humanized nanobody structure that replaces a C2 domain of DLL3, and two DLL3 minimal epitopes inserted into the nanobody structure. NP304 comprises a soluble humanized nanobody structure and two DLL3 minimal epitopes inserted into the nanobody structure. Amino acid sequences of NP296-NP299 and NP302-NP304 are shown in Table 1.

Each of NP247, NP298, NP302, and wild-type control NP116 constructs was separately introduced to H1299 cells by transfection to analyze the expression of engineered DLL3. H1299 cells were then stained with fluorescently labeled anti-DLL3 antibodies and analyzed by FACS to detect DLL3 expression. FIG. 9B shows the amount of DLL3 expressed on the cell surface. Cells expressing NP298, which comprises two DLL3 binding domains, or NP302, which comprises a humanized soluble nanobody domain, showed increased detection level of DLL3 expressed on the cell surface compared to cells expressing NP247 and wild-type control (FIG. 9B).

In addition, each of NP247, NP296, NP298, NP302, NP303, NP304, and control constructs (isotype control and negative control) was separately introduced to H1299 cells. H1299 cells were plated at 40,000 cells/well in a 24-well plate. 24 hours after plating, NP247, NP296NP286, NP298, NP302, NP303, NP304, and control constructs were transfected into the cells via lipofectamine according to the manufacture's protocols. 48 hours post-transfection, cells were harvested and stained with anti-DLL3 antibody followed by an APC-conjugated anti-human antibody for detection. These samples were then read out via flow cytometry on the MACS Quant. FIG. 9C shows the amount of cells stained with anti-DLL3 antibody (% Positive) and GMFI of DLL3-positive cells.

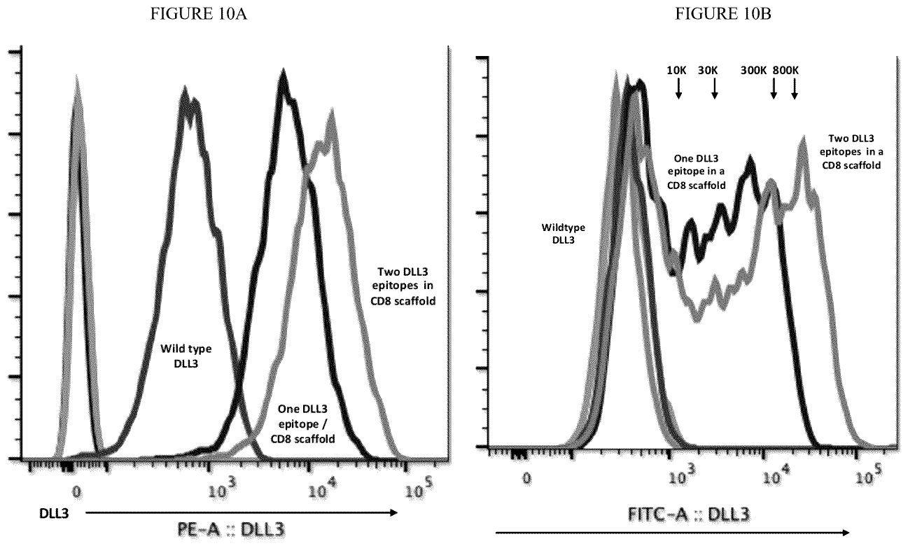

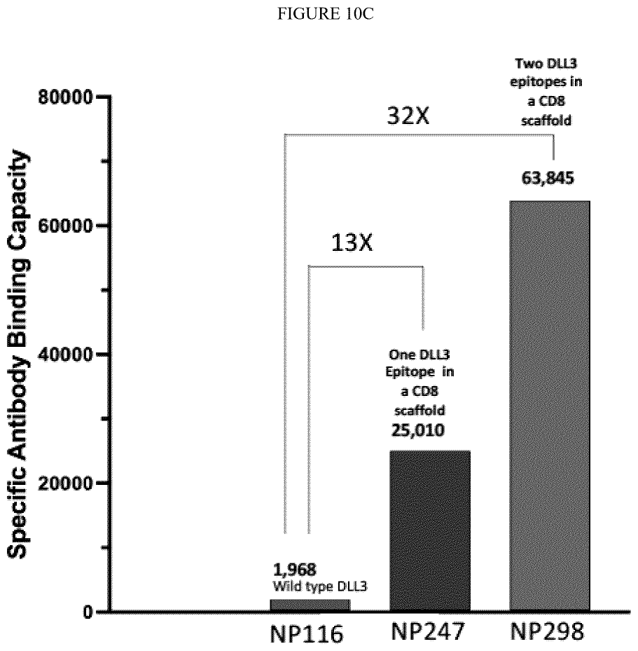

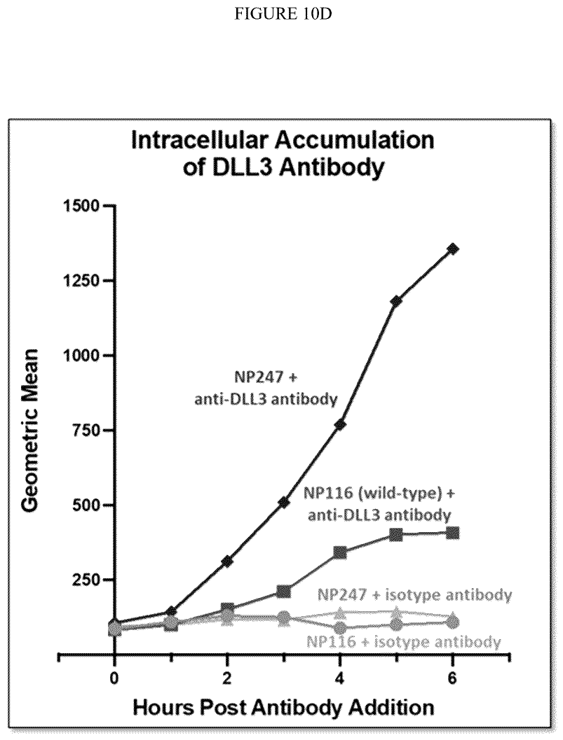

Next, a physical number of copies of anti-DLL3 antibody bound to DLL3 expressed on the cell surface was assessed. H1299 cells were seeded into 96 well plate. 24 hours after seeding, H1299 cells were transfected with nanoplasmid vectors comprising NP116 (wild-type control), NP247 (with a single DLL3 epitope in the CD8 scaffold), or NP298 (with two DLL3 epitopes in the CD8 scaffold) via lipofectamine according to the manufacture's protocols. H1299 cells were then stained with fluorescently labeled anti-DLL3 antibodies for FACS analysis. Cells and commercially available beads for epitope quantification were then stained with an anti-DLL3 secondary antibody conjugated to Alexa Fluor® 647 (AF647). H1299 cells were subsequently analyzed on a BD Biosciences FACSAria IIu (FIGS. 10A-10C). The number of epitopes were calculated by comparing the cell's expression to the quantification bead standard curve (FIGS. 10B-10C). Cells expressing NP247 showed a 13-fold increase in copy number of anti-DLL3 antibodies compared to cells expressing NP116 (FIG. 10C). Cells expressing NP298 showed a 32-fold increase in copy number of anti-DLL3 antibodies compared to cells expressing NP116 (FIG. 10C).

In addition, H1299 cells stably expressing wild-type (NP116) or engineered DLL3 (NP298) were also stained with an anti-DLL3 antibody conjugated to a commercially available internalization secondary reagent (pHrodo). H1299 cells were incubated at 37° C. with 5% CO₂ over a period of six hours. A portion of the samples were removed at regular time intervals and subsequently analyzed on a BD Biosciences FACSAria IIu. FIG. 10D shows the amount of DLL3 internalized.

### Example 6—Positron Emission Tomography (PET) Scan Studies of a Mouse Model System Expressing a Cancer Biomarker

A mouse model system was developed using engineered H1299 cells stably expressing somatostatin receptor 2 (SSTR2). H1299 cell line was established from a lymph node metastasis of the lung from a human patient. The patient had received radiation therapy prior to tumor biopsy. The cell line was adherent and has epithelial morphology. First, a DNA plasmid was designed for expressing a copy of SSTR2 gene. Next, H1299 cells were transfected with the plasmid so that H1299 cells contain two genomic copies of the stably integrated SSTR2 constructs using a 3rd generation lentiviral system (System Biosciences; LL410PA-1). Different numbers of these engineered H1299 cells were then implanted to mice at different locations subcutaneously with the goal of determining the minimal number of cells to produce a measurable signal.

NOD.Cg-Prkdcscid Il2rgtm 1 Wjl/SzJ (NSG) mice were selected from an in-house colony at Stanford University. At 6 weeks of age, mice were anesthetized (isoflurane 3%, in 100% O2) prior to H1299 tumor inoculation. Mice were then placed in a prone position and H1299 WT or H1299-SSTR2 tumors were subcutaneously implanted using a 1:1 (vol/vol) PBS:GelTrex according to Table 2.

TABLE 2
H1299 Tumor Implantation

| | | | Number | | | Total | Syringe |
|---|---|---|---|---|---|---|---|
| Cell | Inoculation | Animal | of | Cells/ | Inoculation | Inoculation | And |
| Line | Site | Strain | Animals | Animal | Medium | Volume | Needle |
| H1299- | SC, right | NSG | 4 | 2.5 × 10⁶ | PBS:Geltrex | 100 μl | 1 ml, |
| WT | shoulder | | | | (1:1) | | 29 G |
| H1299- | SC, left | NSG | 4 | 2.5 × 10⁶ | PBS:Geltrex | 100 μl | 1 ml, |
| SSTR2 | shoulder | | | | (1:1) | | 29 G |

SC: subcutaneous administration

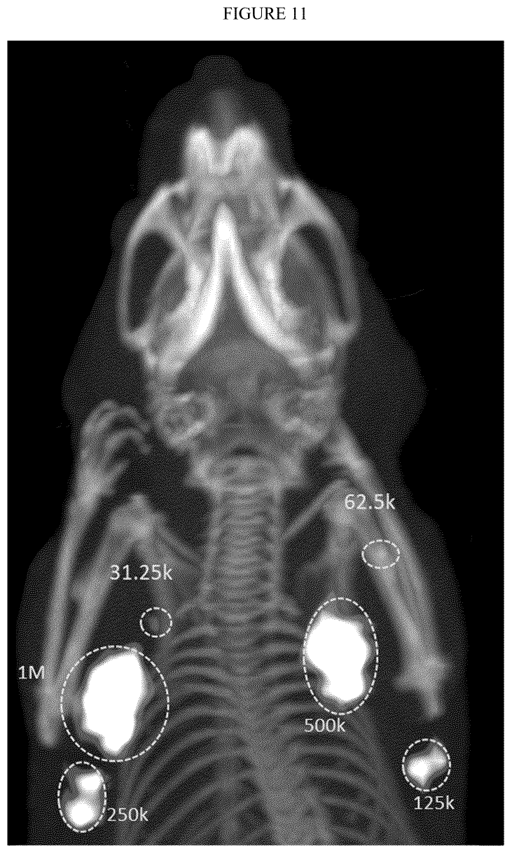

Mice implanted with engineered H1299 cells were then treated with a ⁶⁸Ga-dotate positron-emitting tracer that binds to SSTR2 proteins on the H1299 cell surface. FIG. 11 shows an image taken from a PET scan of this mouse model system. These data illustrate that as little as 31,000 cancer cells can be detected in this mouse model when cells are induced to express the surrogate biomarker (e.g., SSTR2) on the cell surface.

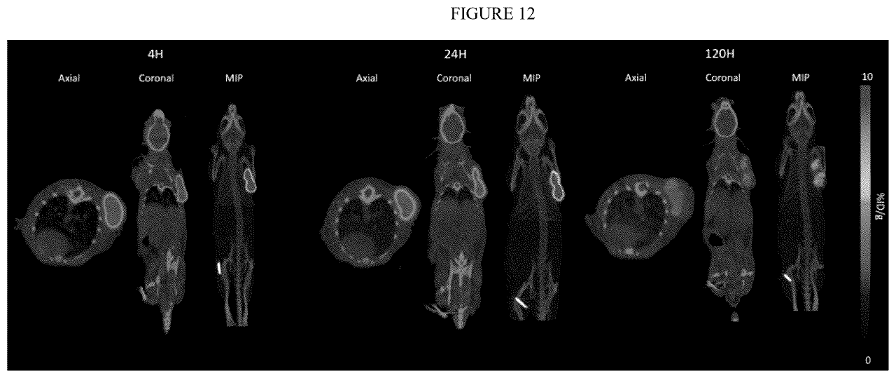

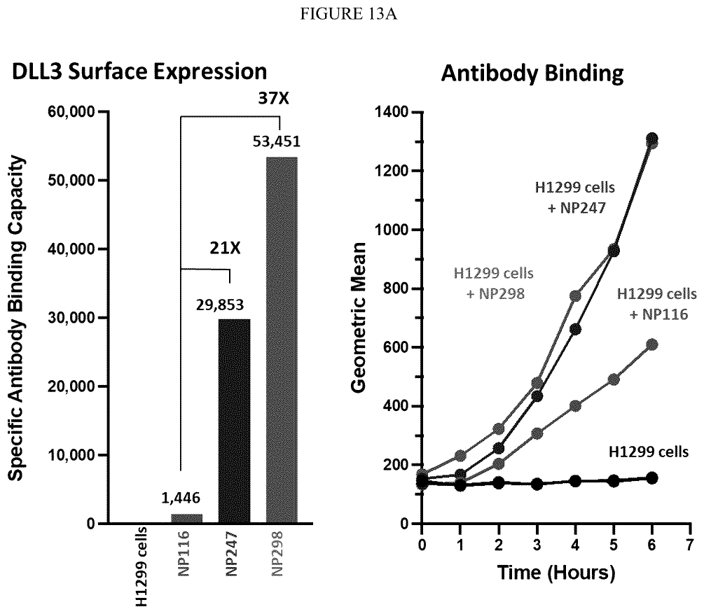

Once the size of tumors reached an average size between 100-200 mm³, mice implanted with engineered H1299 cells were also treated with 200 μCi [¹⁷⁷Lu]-DOTATATE tracer (produced by Minerva, Denmark) that binds to SSTR2 proteins on the H1299 cell surface. SPECT/CT images were obtained 4 hours, 24 hours and 120 hours post tracer administration (FIG. 12). These data illustrate that high energy tracers can be used for the engineered biomarker expression approach.

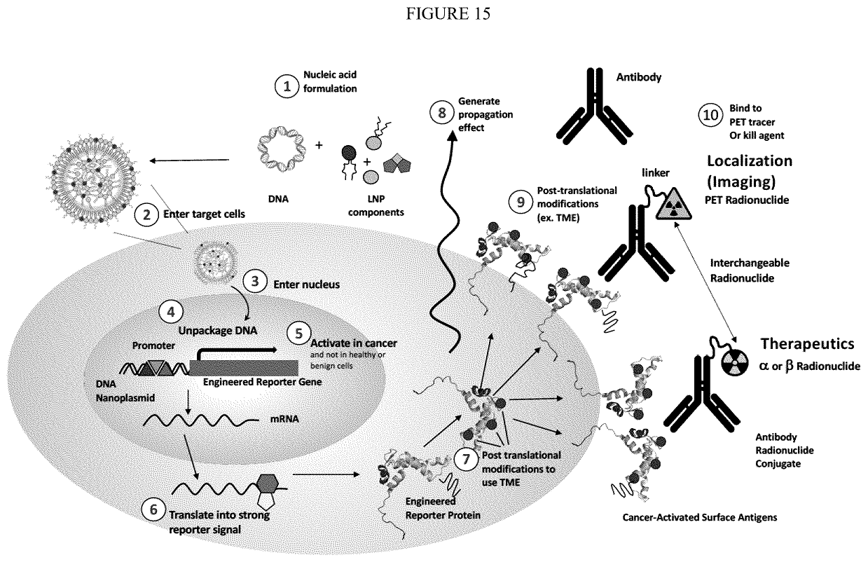

In addition, once the size of tumors reached an average size between 100-200 mm³, mice were dosed with 200 μCi [⁶⁸GA]-DOTATATE tracer (obtained from Cardinal Health) that binds to SSTR2 proteins on the H1299 cell surface. FIG. 15 shows an image taken from a PET/CT scan of this mouse model system 2 hours post tracer administration.

### Example 7—Cell Surface Expression and Cell Killing of Engineered DLL3

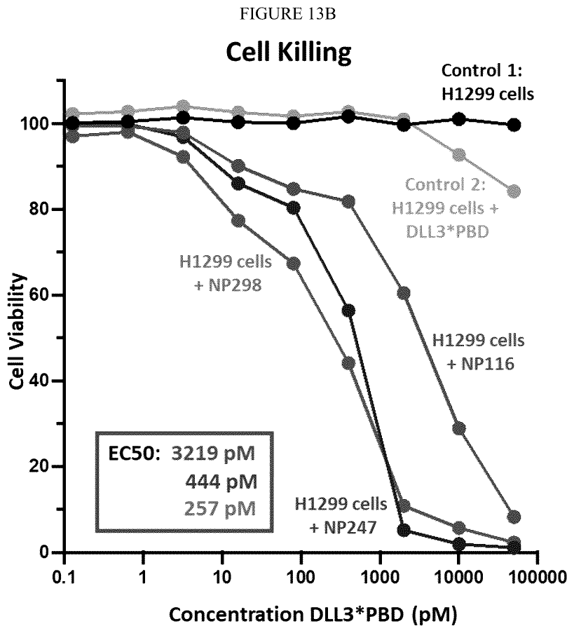

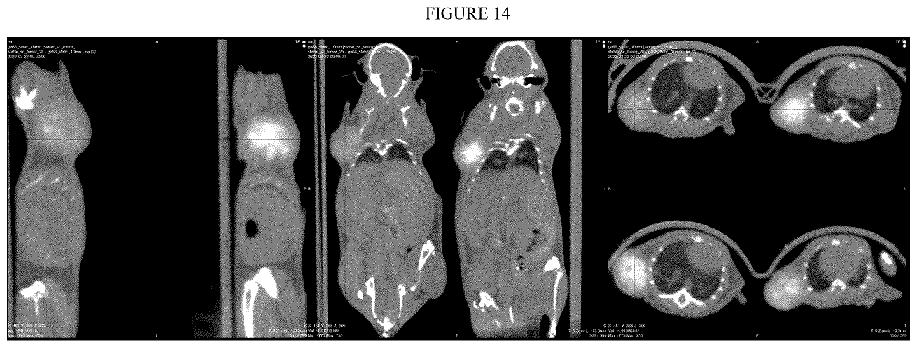

Each of NP247 and NP298 constructs and wild-type DLL3 construct (NP116) was separately introduced to H1299 cells by transfection to analyze the expression of engineered DLL3. H1299 cells were then stained with fluorescently labeled anti-DLL3 antibodies for FACS analysis. H1299 cells stably expressing wild-type or engineered DLL3 and commercially available beads for epitope quantification were then stained with an anti-DLL3 secondary antibody conjugated to Alexa Fluor® 647 (AF647). H1299 cells were subsequently analyzed on a BD Biosciences FACSAria IIu. The number of epitopes were calculated by comparing the cell's expression to the quantification bead standard curve. The same H1299 cells stably expressing wild-type or engineered DLL3 were also stained with an anti-DLL3 antibody conjugated to a commercially available internalization secondary reagent (pHrodo). H1299 cells were incubated at 37° C. with 5% CO₂ over a period of six hours. A portion of the samples were removed at regular time intervals and subsequently analyzed on a BD Biosciences FACSAria Ilu. FIG. 13A shows the amount of DLL3 expressed on the cell surface (left) and the amount of DLL3 internalized (right). Cells expressing NP247 (1 DLL3 epitope) showed a 21-fold increase in copy number of anti-DLL3 antibodies compared to cells expressing NP116 (wild-type DLL3). Cells expressing NP298 (2 DLL3 epitopes) showed 37-fold increase in copy number of anti-DLL3 antibodies compared to cells expressing NP116 (WT DLL3).

Next, H1299 cells stably expressing WT or engineered DLL3 were seeded 5,000 cells/well in 96 well plates. 24 hours after seeding, H1299 cells were treated with a titration of either an unconjugated anti-DLL3 antibody or an anti-DLL3 antibody conjugated to a PBD toxin (anti-DLL3-PBD). 72 hours after antibody treatment, cell viability was assessed using the CellTiter Glo 2.0 assay kit and analyzed on a Promega GloMax Navigator. FIG. 13B shows cells expressing engineered DLL3, NP247 or NP298, that have increased expression level on the cell surface may be more susceptible to cell death when treated with anti-DLL3-PBD compared to cells expressing NP116 (wild-type DLL3).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Embodiments

The following embodiments are intended to be illustrative, and not to be limiting in any way.
1. A method of detecting, imaging, or treating a diseased cell, comprising:
administering to a subject a composition, wherein said composition induces expression of a biomarker preferentially in a diseased cell versus a non-diseased cell, wherein said biomarker comprises an extracellular receptor configured to bind an affinity reagent configured for detection or treatment of said diseased cell.
2. The method of embodiment 1, wherein said extracellular receptor further comprises an activatable epitope, wherein said activatable epitope is preferentially accessible to binding in a physical microenvironment inside or outside of said cell affected by said disease compared to a microenvironment inside or outside of a cell not affected by said disease
3. A method of detecting, imaging, or treating a diseased cell, comprising:
administering to a subject a composition, wherein said composition induces expression of a biomarker preferentially in a diseased cell versus a non-diseased cell,
   wherein said biomarker comprises an activatable epitope,
   wherein said activatable epitope is preferentially accessible to binding in a physical microenvironment inside or outside of said cell affected by said disease compared to a microenvironment inside or outside of a cell not affected by said disease.
4. The method of embodiment 1 or 2, wherein said composition comprises a nucleic acid or vector encoding said biomarker.
5. The method of any one of embodiments 1-4, wherein said biomarker does not comprise a single chain variable fragment (scFv).
6. The method of embodiment 5, wherein said biomarker does not comprise a light chain variable (VL) domain.
7. The method of any one of embodiments 1-6, wherein said biomarker further comprises a pH-sensitive helix.
8. The method of embodiment 7, wherein said activatable epitope is flanked by at least two copies of pH-sensitive helices.
9. The method of embodiment 7 or 8, wherein said pH-sensitive helix comprises a sequence having at least 80% identity to any one of SEQ ID NOs: 1-3.
10. The method of embodiment 8 or 9, wherein said biomarker comprises a sequence according to:
[helix]-[linker]-[helix]-[linker]-[helix]-[linker]-[epitope]
Wherein [helix] denotes said pH-sensitive helix;
[linker] denotes a linker comprising the sequence (GGS)n or (GGS) nFCYWKTCT (GGS)n (SEQ ID NO: 93); and
[epitope] denotes said epitope.
11. The method of embodiment 4 or 9, wherein said biomarker comprises a sequence according to:
[helix]-[linker]-[helix]-[linker]-[helix]-[epitope]
Wherein [helix] denotes said pH-sensitive helix;
[linker] denotes a linker comprising the sequence (GGS)n or RLCRPRSAPSRCGPGLRPCAP (SEQ ID NO: 91); and [epitope] denotes said epitope.
12. A method of detecting, imaging, or treating a diseased cell, comprising:
   administering to a subject a composition, wherein said composition induces surface expression of a biomarker comprising an activatable epitope in a diseased cell preferentially over surface expression of said biomarker in non-diseased cells;
   wherein said activatable epitope is preferentially accessible to binding in a microenvironment specific to said diseased cell compared to a microenvironment of a cell not affected by said disease; wherein said biomarker comprises a membrane-resident polypeptide or wherein said biomarker comprises a polypeptide comprising a transmembrane domain.
13. The method of any one of embodiments 1-12, further comprising administering to said subject an antibody or antigen-binding fragment thereof, a protein ligand or functional fragment thereof, or a small molecule configured to bind said epitope.
14. The method of embodiment 13, further comprising detecting binding of said antibody or antigen binding fragment thereof to said epitope.
15. A method of detecting, imaging, or treating a diseased cell, comprising
(a) administering to a subject a composition, wherein said composition induces surface expression of a biomarker comprising an epitope in a diseased cell preferentially over surface expression of said biomarker in non-diseased cells; and
(b) administering to said subject an antibody or antigen-binding fragment thereof, a protein ligand or functional fragment thereof, or a small molecule configured to bind said epitope.
16. The method of embodiment 15, wherein said extracellular domain does not comprise an scFv.
17. The method of embodiment 16, wherein said extracellular domain does not comprise a VL domain.
18. The method of any one of embodiments 15-17, further comprising detecting binding of said antibody or antigen binding fragment, protein ligand or functional fragment thereof, or small molecule to said epitope.
19. The method of any one of embodiments 15-18, wherein said epitope is an activatable epitope preferentially accessible to binding an extracellular ligand, wherein said preferential accessibility to binding is in a microenvironment specific to said diseased cell compared to an intracellular or extracellular microenvironment of a cell not affected by said disease.
20. The method of embodiment 19, wherein said activatable epitope is activated via phosphorylation, glycosylation, methylation, ubiquitinylation, α-acetylation, lipidation, lipidation by glycosylphosphatidylinositol, amidation, sulfonation, oxidation, or any combination thereof.
21. The method of any one of embodiments 15-20, wherein said biomarker further comprises a pH-sensitive helix.
22. The method of embodiment 21, wherein said activatable epitope is flanked by at least two copies of pH-sensitive helices.
23. The method of embodiment 21 or 22, wherein said pH-sensitive helix comprises a sequence having at least 80% identity to any one of SEQ ID NOs: 1-3.
24. The method of any one of embodiments 21-23, wherein said biomarker comprises a sequence according to:
[helix]-[linker]-[helix]-[linker]-[helix]-[linker]-[epitope]
Wherein [helix] denotes said pH-sensitive helix;
[linker] denotes a linker comprising the sequence (GGS)n or (GGS) nFCYWKTCT (GGS)n (SEQ ID NO: 93); and [epitope] denotes said epitope.
25. The method of any one of embodiments 21-23, wherein said biomarker comprises a sequence according to:
[helix]-[linker]-[helix]-[linker]-[helix]-[epitope]
Wherein [helix] denotes said pH-sensitive helix;
[linker] denotes a linker comprising the sequence (GGS)n or RLCRPRSAPSRCGPGLRPCAP (SEQ ID NO: 91); and [epitope] denotes said epitope.
26. The method of embodiment 16, wherein said biomarker further comprises a protease cleavage site for a cancer-specific protease alongside an scFv configured to bind said activatable epitope.
27. The method of embodiment 26, wherein said cancer-specific protease is uPA, EPCAM, an MMP, a cathepsin, a serine protease, a granzyme, a furin, a cysteine protease, an elastase, or a type II transmembrane serine protease, Kallikrein-related peptidases, ‘a disintegrin and metalloproteinase’ (ADAM), plasminogen activators, serine protease plasminogen activators, a caspase, or any combination thereof.
28. The method of any one of embodiments 13-27, wherein said antibody or antigen-binding fragment thereof further comprises a radioisotope.
29. The method of embodiment 20, wherein said radioisotope comprises a positron-emitting radioisotope, an alpha-emitting radioisotope, a beta-emitting radioisotope, or 99mTc.
30. The method of embodiment 29, wherein said radioisotope is a positron-emitting radioisotope and comprises ¹²⁴I, ⁶⁸Ga ¹¹C, 13N, ¹⁵O, ¹⁸F, 68Ga, 64Cu, 52Mn, 55Co, 89Zr, 82Rb, or any combination thereof.
31. The method of embodiment 29, wherein said radioisotope comprises an alpha-emitting radioisotope and comprises 225 Ac, 211 At, 227Th, 224Ra, or any combination thereof.
32. The method of embodiment 29, wherein said radioisotope comprises a beta-emitting radioisotope and comprises 177Lu, 67Cu, ¹³¹I, ⁹⁰Y, 89Sr, 186Re, 165Dy, 32P, 166Ho, 188Re, or any combination thereof.
33. The method of any one of embodiments 1-32, wherein said biomarker further comprises an extracellular domain comprising said epitope.
34. The method of embodiment 33, wherein said extracellular domain is derived from a membrane protein endogenous to said subject.
35. The method of embodiment 33, wherein said extracellular domain is derived from 158, PSMA, SSTR2, CD8a, CD4, TGFR1, IGF1R, PD-L1, EGFR, CD73, IL-6, or any combination thereof.
36. The method of embodiment 35, wherein said extracellular domain is derived from CD8a, CD4, TGFR1, IGF1R, PD-L1, EGFR, CD73, IgK, or any combination thereof.
37. The method of embodiment 35 or 36, wherein said extracellular domain further comprises an activatable epitope derived from DLL3, PSMA, SSTR2, or any combination thereof.
38. The method of any one of embodiments 1-37, wherein said biomarker comprises an extracellular domain comprising a sequence having at least 80% identity to any one of SEQ ID NOs: 4-9, a functional fragment thereof, or a variant thereof.
39. The method of any one of embodiments 1-38, wherein said biomarker further comprises a hinge domain derived from IgG4, IgG1, CD8, or CD28, or the sequence (GGGS)n (SEQ ID NO: 92).
40. The method of any one of embodiments 1-39, wherein said biomarker further comprises a transmembrane domain derived from DLL3, PSMA, SSTR2, PD-L1, EGFR, CD28, CD4, CD8a, ICOS, or CD73.
41. The method of any one of embodiments 1-40, wherein said biomarker further comprises an intracellular domain comprising a reporter protein or an affinity tag.
42. The method of embodiment 41, wherein said biomarker further comprises an intracellular domain comprising a reporter protein, wherein said reporter protein is derived from GFP, a luciferase, Nluc, or Fluc, or any combination thereof.
43. The method of embodiment 41, wherein said biomarker further comprises an intracellular domain comprising an affinity tag, wherein said affinity tag comprises an HA, FLAG, or MYC sequence, or any combination thereof.
44. The method of any one of embodiments 1-43, wherein said biomarker comprises more than one copy of said epitope.
45. The method of any one of embodiments 1-44, wherein said composition comprises a nucleic acid vector comprising a coding sequence for said biomarker.
46. The method of embodiment 45, wherein said coding sequence comprises a promoter operably linked to a nucleotide sequence encoding the biomarker.
47. The method of embodiment 46, wherein said promoter drives expression of said biomarker in said diseased cell preferentially relative to expression of said biomarker in a non-diseased cell in said subject.
48. The method of embodiment 46 or 47, wherein said promoter comprises a promoter of a gene selectively overexpressed in a diseased cell versus a non-diseased cell, or a functional fragment thereof.
49. The method of any one of embodiments 46-48, wherein said promoter is selected from the group consisting of a Survivin promoter (BIRC5), a CXCR4 promoter, an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransferase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11A1) promoter, a collagen type I alpha 1 chain (COL1A1) promoter, a cystatin SN (CST1) promoter, a denticleless E3 ubiquitin protein ligase homolog (DTL) promoter, a family with sequence similarity 111 member B (FAM111B) promoter, a forkhead box A1 (FOXA1) promoter, a kinesin family member 20A (KIF20A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2A member 1 (SCGB2A1) promoter, topoisomerase II alpha (TOP2A) promoter, a ubiquitin D (UBD) promoter, a ubiquitin conjugating enzyme E2 C (UBE2C), a USH1 protein network component harmonin (USH1C), a V-set domain containing T cell activation inhibitor 1 (VTCN1) promoter, a Hexokinase type II promoter, a TRPM4 promoter, a stromelysin 3 promoter, a surfactant protein A promoter, a secretory leukoprotease inhibitor promoter, a tyrosinase promoter, a stress-inducible grp78/BiP promoter, an interleukin-10 promoter, an α-B-crystallin/heat shock protein 27 promoter, an epidermal growth factor receptor promoter, a mucin-like glycoprotein promoter, an mts1 promoter, an NSE promoter, a somatostatin receptor promoter, a c-erbB-3 promoter, a c-erbB-2 promoter, a c-erbB4 promoter, a thyroglobulin promoter, an α-fetoprotein promoter, a villin promoter, an albumin promoter, a glycoprotein A33 promoter, the B cell-specific Moloney leukemia virus insertion site 1 promoter, a cyclooxygenase-2 promoter, a fibroblast growth factor promoter; a human epidermal growth factor receptor 2, a human telomerase reverse transcriptase promoter; a kinase domain insert containing receptor promoter; a rad51 recombinase promoter; TTF-1, an urokinase-type plasminogen activator receptor promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (UBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box M1 (FOXM1) promoter, a cell proliferation regulating inhibitor of protein phosphatase 2A (KIAA1524) promoter, a kinesin family member 2C (KIF2C) promoter, a karyopherin subunit alpha 2 (KPNA2) promoter, a MYB proto-oncogene like 2 (MYBL2) promoter, a NIMA related kinase 2 (NEK2) promoter, a RAN binding protein 1 (RANBP1) promoter, a small nuclear ribonucleoprotein polypeptides B and B1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17orf53) promoter, a chromobox 3 (CBX30) promoter, a cyclin B1 (CCNB1) promoter, a cyclin E1 (CCNE1) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL10A1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18A (KIF18A) promoter, a kinesin family member 4A (KIF4A) promoter, a kinesin family member C1 (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, a kinetochore complex component (NDC80) promoter, a nudix hydrolase 1 (NUDT1) promoter, a ribonuclease H2 subunit A (RNASEH2A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP13) promoter, a trophinin associated protein (TROAP) promoter, a ubiquitin conjugating enzyme E2 C (UBE2C) promoter, a WD repeat and HMG-box DNA binding protein 1 (WDHD1) promoter, an alpha fetoprotein (AFP) promoter, a fragment thereof, any combination thereof, a chimeric promoter compiled from a plurality of elements from the aforementioned, or a fully synthetic promoter composed of tiled transcription-factor binding sites.
50. The method of any one of embodiments 45-49, wherein said vector is a recombinant viral vector.
51. The method of any one of embodiments 45-49, wherein said vector is a non-viral vector.
52. The method of embodiment 51, wherein said non-viral vector is a nanoplasmid, a plasmid, a minicircle, a close-ended linear duplex (CELiD), or a doggybone DNA vector (dbDNA).
53. The method of any one of embodiments 1-52, wherein said composition further comprises a transfection agent.
54. The method of embodiment 53, wherein said transfection agent is a linear or branched polyethylenimine, nanoparticle, lipophilic particle, solid nanoparticle, peptide, micelle, dendrimer, polymeric composition, hydrogel, synthetic or naturally derived exosome, virus-like particles, or any combination thereof.
55. The method of any one of embodiments 1-54, wherein said disease is a cancer, an autoimmune disease, or a neurodegenerative disease.
56. The method of embodiment 55, wherein said disease is a cancer.
57. The method of embodiment 56, wherein said cancer comprises Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, Uveal Melanoma, lip melanoma, spindle cell carcinoma, liposarcoma, nasal sarcoma, mammary adenocarcinoma, insulinoma, osteosarcoma, mast cell tumors, hemangiosarcoma, non-small cell lung carcinoma (NSCLC), marginal lymphoma, malignant melanoma, or chronic lymphocytic leukemia.
58. The method of any one of embodiments 14-57, wherein said detecting binding of said antibody or antigen binding fragment thereof to said epitope comprises a PET imaging method or another radioisotope-based imaging method.
59. A nucleic acid comprising:
a cancer-specific promoter operably linked to a nucleotide sequence encoding a membrane-resident polypeptide biomarker comprising an activatable epitope.
60. The method of embodiment 59, wherein said extracellular domain does not comprise an scFv.
61. The method of embodiment 60, wherein said extracellular domain does not comprise a VL domain.
62. The nucleic acid of any one of embodiments 59-61, wherein said activatable epitope is activated via phosphorylation, glycosylation, methylation, ubiquitinylation, A-acetylation, lipidation (eg GPI), amidation, sulfonation, oxidation, or any combination thereof
63. The nucleic acid of any one of embodiments 59-62, wherein said biomarker further comprises an extracellular domain comprising said epitope and a transmembrane domain.
64. The nucleic acid of any one of embodiments 59-63 wherein said biomarker further comprises a pH-sensitive helix.
65. The nucleic acid any one of embodiments 59-64, wherein said activatable epitope is flanked by at least two copies of pH-sensitive helices.
66. The nucleic acid of any one of embodiments 59-65, wherein said pH-sensitive helix comprises a sequence having at least 80% identity to any one of SEQ ID NOs: 1-3.
67. The nucleic acid of any one of embodiments 59-66, wherein said biomarker comprises a sequence according to:
[helix]-[linker]-[helix]-[linker]-[helix]-[linker]-[epitope]
Wherein [helix] denotes said pH-sensitive helix;
[linker] denotes a linker comprising the sequence (GGS)n or (GGS) nFCYWKTCT (GGS)n (SEQ ID NO: 93); and
[epitope] denotes said epitope.
68. The nucleic acid of any one of embodiments 59-66, wherein said biomarker comprises a sequence according to:
[helix]-[linker]-[helix]-[linker]-[helix]-[epitope]
Wherein [helix] denotes said pH-sensitive helix;
[linker] denotes a linker comprising the sequence (GGS)n or RLCRPRSAPSRCGPGLRPCAP (SEQ ID NO: 91); and
[epitope] denotes said epitope.
69. The nucleic acid of any one of embodiments 59-68, wherein said biomarker further comprises a protease cleavage site for a cancer-specific protease alongside an scFv configured to bind said activatable epitope.
70. The nucleic acid of embodiment 67, wherein said cancer-specific protease is uPA, EPCAM, an MMP, a cathepsin, a serine protease, a granzyme, a furin, a cysteine protease, an elastase, or a type II transmembrane serine protease, Kallikrein-related peptidases, ‘a disintegrin and metalloproteinase’ (ADAM), plasminogen activators (a serine protease), a caspase, or any combination thereof.
71. The nucleic acid of any one of embodiments 60-70, wherein said extracellular domain is derived from a membrane protein endogenous to said subject.
72. The nucleic acid of any one of embodiments 60-71, wherein said extracellular domain is derived from DLL3, PSMA, SSTR2, CD8a, CD4, TGFR1, IGF1R, PD-L1, EGFR, CD73, IgK, or IL-6, or any combination thereof.
73. The nucleic acid of embodiment 72, wherein said extracellular domain is derived from CD8a, CD4, TGFR1, IGF1R, PD-L1, EGFR, CD73, IgK, or any combination thereof.
74. The nucleic acid of embodiment 72 or 73, wherein said extracellular domain further comprises an activatable epitope derived from DLL3, PSMA, SSTR2, or any combination thereof.
75. The nucleic acid of any one of embodiments 60-74, wherein said biomarker comprises an extracellular domain comprising a sequence having at least 80% identity to any one of SEQ ID NOs: 4-9, a functional fragment thereof, or a variant thereof.
76. The nucleic acid of any one of embodiments 59-75, wherein said biomarker further comprises a hinge domain derived from an Ig-superfamily receptor, or comprises the sequence (GGGS)n (SEQ ID NO: 92).
77. The nucleic acid of embodiment 76, wherein said biomarker further comprises said hinge domain, and said hinge domain is derived from IgG4, IgG1, CD8, or CD28.
78. The nucleic acid of any one of embodiments 60-77, wherein said biomarker further comprises a transmembrane domain derived from DLL3, PSMA, SSTR2, PD-L1, EGFR, CD28, CD4, CD8a, ICOS, or CD73.
79. The nucleic acid of any one of embodiments 59-78, wherein said biomarker further comprises an intracellular domain comprising a reporter protein or an affinity tag.
80. The nucleic acid of embodiment 79, wherein said biomarker further comprises an intracellular domain comprising a reporter protein, wherein said reporter protein is derived from GFP, a luciferase, Nluc, or Fluc, or any combination thereof.
81. The nucleic acid of embodiment 79, wherein said biomarker further comprises an intracellular domain comprising an affinity tag, wherein said affinity tag comprises an HA, FLAG, or MYC sequence, or any combination thereof.
82. The nucleic acid of any one of embodiment 59-81, wherein said promoter is selected from the group consisting of a Survivin promoter (BIRC5), a CXCR4 promoter, an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransferase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11A1) promoter, a collagen type I alpha 1 chain (COL1A1) promoter, a cystatin SN (CST1) promoter, a denticleless E3 ubiquitin protein ligase homolog (DTL) promoter, a family with sequence similarity 111 member B (FAM111B) promoter, a forkhead box A1 (FOXA1) promoter, a kinesin family member 20A (KIF20A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2A member 1 (SCGB2A1) promoter, topoisomerase II alpha (TOP2A) promoter, a ubiquitin D (UBD) promoter, a ubiquitin conjugating enzyme E2 C (UBE2C), a USH1 protein network component harmonin (USH1C), a V-set domain containing T cell activation inhibitor 1 (VTCN1) promoter, a Hexokinase type II promoter, a TRPM4 promoter, a stromelysin 3 promoter, a surfactant protein A promoter, a secretory leukoprotease inhibitor promoter, a tyrosinase promoter, a stress-inducible grp78/BiP promoter, an interleukin-10 promoter, an α-B-crystallin/heat shock protein 27 promoter, an epidermal growth factor receptor promoter, a mucin-like glycoprotein promoter, an mts1 promoter, an NSE promoter, a somatostatin receptor promoter, a c-erbB-3 promoter, a c-erbB-2 promoter, a c-erbB4 promoter, a thyroglobulin promoter, an α-fetoprotein promoter, a villin promoter, an albumin promoter, a glycoprotein A33 promoter, the B cell-specific Moloney leukemia virus insertion site 1 promoter, a cyclooxygenase-2 promoter, a fibroblast growth factor promoter; a human epidermal growth factor receptor 2, a human telomerase reverse transcriptase promoter; a kinase domain insert containing receptor promoter; a rad51 recombinase promoter; TTF-1, an urokinase-type plasminogen activator receptor promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (UBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box M1 (FOXM1) promoter, a cell proliferation regulating inhibitor of protein phosphatase 2A (KIAA1524) promoter, a kinesin family member 2C (KIF2C) promoter, a karyopherin subunit alpha 2 (KPNA2) promoter, a MYB proto-oncogene like 2 (MYBL2) promoter, a NIMA related kinase 2 (NEK2) promoter, a RAN binding protein 1 (RANBP1) promoter, a small nuclear ribonucleoprotein polypeptides B and B1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17orf53) promoter, a chromobox 3 (CBX30) promoter, a cyclin B1 (CCNB1) promoter, a cyclin E1 (CCNE1) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL10A1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18A (KIF18A) promoter, a kinesin family member 4A (KIF4A) promoter, a kinesin family member C1 (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, a kinetochore complex component (NDC80) promoter, a nudix hydrolase 1 (NUDT1) promoter, a ribonuclease H2 subunit A (RNASEH2A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP13) promoter, a trophinin associated protein (TROAP) promoter, a ubiquitin conjugating enzyme E2 C (UBE2C) promoter, a WD repeat and HMG-box DNA binding protein 1 (WDHD1) promoter, an alpha fetoprotein (AFP) promoter, a fragment thereof, any combination thereof, a chimeric promoter compiled from a plurality of elements from the aforementioned, or a fully synthetic promoter composed of tiled transcription-factor binding sites.
83. The nucleic acid of any one of embodiments 59-82, wherein said nucleic acid comprises RNA or a capped mRNA.
84. The nucleic acid of embodiment 83, wherein said RNA or said capped mRNA comprises a splice signal, a synthetic intron, an miRNA binding site, a synthetic stem-loop or tetraloop, or an aRNA domain.
85. A vector comprising the nucleic acid of any one of embodiments 59-84.
86. The vector of embodiment 85, wherein said vector is a recombinant viral vector.
87. The vector of embodiment 85, wherein said vector is a non-viral vector.
88. The vector of embodiment 87, wherein said non-viral vector is a nanoplasmid, a plasmid, a minicircle, a close-ended linear duplex (CELiD), or a Doggybone DNA vector (dbDNA).
89. A composition comprising the vector of any one of embodiment 83-88 or the nucleic acid of any one of embodiments embodiment 59-84 and a transfection agent.
90. The composition of embodiment 89, wherein said transfection agent is a linear or branched polyethylenimine, nanoparticle, lipophilic particle, solid nanoparticle, peptide, micelle, dendrimer, polymeric composition, hydrogel, synthetic or naturally derived exosome, virus-like particles, or any combination thereof.
91. A cell comprising the nucleic acid of any one of embodiment 59-84 or the vector of any one of embodiments 84-88.
92. A method of detecting, imaging, or treating a diseased cell, comprising:
   administering to a subject a composition which induces expression of a secretable biomarker preferentially in a diseased cell versus a non-diseased cell, wherein said biomarker comprises:
   (a) an extracellular domain configured to bind an affinity reagent configured for detection or treatment of said diseased cell; and
   (b) a membrane affinity domain capable of associating with, inserting into or binding the outer membrane of a cell.
93. The method of embodiment 92, wherein said extracellular domain does not comprise an scFv.
94. The method of embodiment 93, wherein said extracellular domain does not comprise a VL domain.
95. The method of any one of embodiments 92-94, wherein said composition comprises a nucleic acid or vector, wherein said nucleic acid or vector encodes said secretable biomarker.
96. The method of embodiment any one of embodiments 92-95, wherein said biomarker comprises (a) and (b) in N- to C-terminal order.
97. The method of any one of embodiments 92-96, wherein said extracellular domain comprises an extracellular domain of an extracellular receptor, an epitope thereof, or a ligand-binding domain thereof.
98. The method of any one of embodiments 92-97, wherein said extracellular receptor is DLL3, PSMA, SSTR2, CD8a, CD4, TGFR1, IGF1R, PD-L1, EGFR, CD73, IgK, or IL-6, or any combination thereof.
99. The method of any one of embodiments 92-98, wherein said extracellular domain comprises a sequence having at least 80% sequence identity to any one of SEQ ID NOs: 4-6 or 17-19, a functional fragment thereof, or a variant thereof.
100. The method of any one of embodiments 92-99, wherein said biomarker is encoded by an open reading frame encoding an N-terminal cleavable signal peptide.
101. The method of any one of embodiments 92-100, wherein said membrane affinity domain is configured to bind a cell membrane at low pH or in the presence of a cancer-specific extracellular protease.
102. The method of embodiment 101, wherein said membrane affinity domain is configured to bind a cell membrane at low pH, and said affinity domain comprises a sequence having at least 80% sequence identity to any one of SEQ ID NOs: 10-16, or a variant thereof.
103. The method any one of embodiments 92-102, wherein said membrane affinity domain comprises a C1, C2, PH, FYVE, PX, or ENTH domain, or a transmembrane helix derived from bacteriorhodopsin.
104. The method of any one of embodiments 92-103, further comprising
   (c) administering to said subject an antibody or antigen-binding derivative or fragment thereof, a protein ligand or functional fragment thereof, or a small molecule configured to bind said extracellular domain.
105. The method of embodiment 104, further comprising
   (d) detecting binding of said antibody or antigen-binding derivative or fragment thereof, said protein ligand or functional fragment thereof, or said small molecule to said extracellular domain.
106. The method of embodiment 105, wherein detecting binding of said antibody or antigen-binding derivative or fragment thereof, said protein ligand or functional fragment thereof, or said small molecule to said extracellular domain comprises a PET imaging method or another radioisotope-based imaging method.
107. The method of any one of embodiments 93-106, wherein said composition comprises said vector, wherein said vector is a recombinant viral vector.
108. The method of any one of embodiments 93-107, wherein said vector is a non-viral vector.
109. The method of embodiment 108, wherein said non-viral vector is a nanoplasmid, a plasmid, a minicircle, a close-ended linear duplex (CELiD), or a doggybone DNA vector (dbDNA).
110. The method of any one of embodiments 92-109, wherein said disease is a cancer.
111. A nucleic acid comprising an open reading frame (ORF) encoding a polypeptide, wherein said polypeptide comprises:
   (a) an N-terminal cleavable signal peptide;
   (b) an extracellular domain configured to bind an affinity reagent, wherein said extracellular domain comprises an extracellular domain of an extracellular receptor, an epitope thereof, or a ligand-binding domain thereof; and
   (c) a membrane affinity domain capable of associating with, inserting into, or binding the outer membrane of a cell.
112. The nucleic acid of embodiment 111, wherein said extracellular domain does not comprise an scFv.
113. The nucleic acid of embodiment 112, wherein said extracellular domain does not comprise a VL domain.
114. The nucleic acid of any one of embodiments 111-113, wherein said extracellular receptor is DLL3, PSMA, SSTR2, CD8a, CD4, TGFR1, IGF1R, PD-L1, EGFR, CD73, IgK, or IL-6, or any combination thereof.
115. The nucleic acid of any one of embodiments 111-114, wherein said membrane affinity domain comprises a C1, C2, PH, FYVE, PX, or ENTH domain, or a transmembrane helix derived from bacteriorhodopsin.
116. A nucleic acid comprising an open reading frame (ORF) encoding a polypeptide, wherein said polypeptide comprises:
(a) an extracellular domain configured to bind an affinity reagent, wherein said extracellular domain comprises an extracellular domain of an extracellular receptor, an epitope thereof, or a ligand-binding domain thereof; wherein said wherein said extracellular receptor is DLL3, PSMA, SSTR2, CD8a, CD4, TGFR1, CD73, or IgK, or any combination thereof; and
(b) a membrane affinity domain capable of associating with, inserting into, or binding the outer membrane of a cell.
117. The nucleic acid of embodiment 116, wherein said extracellular domain does not comprise an scFv.
118. The nucleic acid of embodiment 117, wherein said extracellular domain does not comprise a VL domain.
119. The nucleic acid of any one of embodiments 116-118, wherein said membrane affinity domain comprises a C1, C2, PH, FYVE, PX, or ENTH domain, or a transmembrane helix derived from bacteriorhodopsin.
120. A nucleic acid comprising an open reading frame (ORF) encoding a polypeptide, wherein said polypeptide comprises:
   (a) an extracellular domain configured to bind an affinity reagent, wherein said extracellular domain comprises an extracellular domain of an extracellular receptor, an epitope thereof, or a ligand-binding domain thereof; and
   (b) a membrane affinity domain capable of associating with or binding the outer membrane of a cell, wherein said membrane affinity domain comprises a C1, C2, PH, FYVE, PX, or ENTH domain.
121. The nucleic acid of embodiment 120, wherein said extracellular domain does not comprise an scFv.
122. The nucleic acid of embodiment 121, wherein said extracellular domain does not comprise a VL domain.
123. The nucleic acid of any one of embodiments 120-122, wherein said extracellular receptor is DLL3, PSMA, SSTR2, CD8a, CD4, TGFR1, IGF1R, PD-L1, EGFR, CD73, IgK, or IL-6, or any combination thereof.
124. The nucleic acid of any one of embodiments 111-121, wherein said extracellular domain comprises a sequence having at least 80% sequence identity to any one of SEQ ID NOs: 4-6 or 17-19, a functional fragment thereof, or a variant thereof.
125. The nucleic acid of any one of embodiments 116-124, wherein said polypeptide comprises N-terminal cleavable signal peptide.
126. The nucleic acid of any one of embodiments 111-125, wherein said membrane affinity domain is configured to bind a cell membrane at low pH or in the presence of a cancer-specific extracellular protease.
127. The nucleic acid of embodiment 126, wherein said membrane affinity domain is configured to bind a cell membrane at low pH, and said affinity domain comprises a sequence having at least 80% sequence identity to any one of SEQ ID NOs: 10-16, or a variant thereof.
128. The nucleic acid of any one of embodiments 111-127, wherein the polypeptide further comprises a hinge domain from an Ig-superfamily receptor between said extracellular domain and said membrane affinity domain.
129. The nucleic acid of any one of embodiments 111-127, wherein said ORF is codon-optimized for expression in a mammalian cell.
130. The nucleic acid of any one of embodiments 111-129, further comprising 167 ng a cancer-specific promoter, wherein said ORF is operably linked to said cancer-specific promoter.
131. The nucleic acid of embodiment 130, wherein said cancer-specific promoter is a promoter of a gene overexpressed in a cancer cell versus a normal cell, or a functional fragment thereof.
132. The nucleic acid of embodiment 130 or 131, wherein said promoter is comprises a Survivin promoter (BIRC5), a CXCR4 promoter, an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransferase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11A1) promoter, a collagen type I alpha 1 chain (COL1A1) promoter, a cystatin SN (CST1) promoter, a denticleless E3 ubiquitin protein ligase homolog (DTL) promoter, a family with sequence similarity 111 member B (FAM111B) promoter, a forkhead box A1 (FOXA1) promoter, a kinesin family member 20A (KIF20A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2A member 1 (SCGB2A1) promoter, topoisomerase II alpha (TOP2A) promoter, a ubiquitin D (UBD) promoter, a ubiquitin conjugating enzyme E2 C (UBE2C), a USH1 protein network component harmonin (USH1C), a V-set domain containing T cell activation inhibitor 1 (VTCN1) promoter, a Hexokinase type II promoter, a TRPM4 promoter, a stromelysin 3 promoter, a surfactant protein A promoter, a secretory leukoprotease inhibitor promoter, a tyrosinase promoter, a stress-inducible grp78/BiP promoter, an interleukin-10 promoter, an α-B-crystallin/heat shock protein 27 promoter, an epidermal growth factor receptor promoter, a mucin-like glycoprotein promoter, an mts1 promoter, an NSE promoter, a somatostatin receptor promoter, a c-erbB-3 promoter, a c-erbB-2 promoter, a c-erbB4 promoter, a thyroglobulin promoter, an α-fetoprotein promoter, a villin promoter, an albumin promoter, a glycoprotein A33 promoter, the B cell-specific Moloney leukemia virus insertion site 1 promoter, a cyclooxygenase-2 promoter, a fibroblast growth factor promoter; a human epidermal growth factor receptor 2, a human telomerase reverse transcriptase promoter; a kinase domain insert containing receptor promoter; a rad51 recombinase promoter; TTF-1, an urokinase-type plasminogen activator receptor promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (UBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box M1 (FOXM1) promoter, a cell proliferation regulating inhibitor of protein phosphatase 2A (KIAA1524) promoter, a kinesin family member 2C (KIF2C) promoter, a karyopherin subunit alpha 2 (KPNA2) promoter, a MYB proto-oncogene like 2 (MYBL2) promoter, a NIMA related kinase 2 (NEK2) promoter, a RAN binding protein 1 (RANBP1) promoter, a small nuclear ribonucleoprotein polypeptides B and B1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17orf53) promoter, a chromobox 3 (CBX30) promoter, a cyclin B1 (CCNB1) promoter, a cyclin E1 (CCNE1) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL10A1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18A (KIF18A) promoter, a kinesin family member 4A (KIF4A) promoter, a kinesin family member C1 (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, a kinetochore complex component (NDC80) promoter, a nudix hydrolase 1 (NUDT1) promoter, a ribonuclease H2 subunit A (RNASEH2A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP13) promoter, a trophinin associated protein (TROAP) promoter, a ubiquitin conjugating enzyme E2 C (UBE2C) promoter, a WD repeat and HMG-box DNA binding protein 1 (WDHD1) promoter, an alpha fetoprotein (AFP) promoter, a functional fragment thereof, any combination thereof, a chimeric promoter compiled from a plurality of elements from the aforementioned, or a fully synthetic promoter composed of tiled transcription-factor binding sites derived from any of the aforementioned.
133. A vector comprising the nucleic acid of any one of embodiments 111-132.
134. The vector of embodiment 133, wherein said vector is a recombinant viral vector.
135. The vector of embodiment 133, wherein said vector is a non-viral vector.
136. An engineered polypeptide, comprising:
   (a) an extracellularly-oriented domain comprising an epitope capable of binding: (i) an antibody or (ii) a peptide hormone or growth factor, wherein said extracellularly-oriented domain does not comprise an scFv;
   (b) an extracellularly-oriented polypeptide spacer domain of about 15 to about 40 angstroms in length when folded; and
   (c) a transmembrane domain or a membrane affinity domain capable of associating with an outer membrane of a cell, wherein at least two of (a), (b), and (c) are heterologous to each other.
137. The engineered polypeptide of embodiment 136, wherein said polypeptide does not comprise a light chain variable (VL) domain.
138. The engineered polypeptide of embodiment 136 or 137, wherein said engineered polypeptide does not comprise an intracellular signaling domain.
139. The engineered polypeptide of any one of embodiments 136-138, wherein said engineered polypeptide does not comprise an intracellular portion of a CD3zeta, CD137, or CD28 polypeptide.
140. The engineered polypeptide of any one of embodiments 136-139, wherein an intracellularly-oriented portion of said polypeptide comprises less than or equal to 100, 75, 50, 25, 10, or 5 residues.
141. The engineered polypeptide of any one of embodiments 136-140, wherein said extracellularly-oriented polypeptide spacer domain comprises a hinge domain.
142. The engineered polypeptide of any one of embodiments 136-141, where (a), (b), and (c) are in order from N- to C-terminus of said engineered polypeptide.
143. The engineered polypeptide of any one of embodiments 136-142, wherein said engineered polypeptide is capable of display on a cell surface.
144. The engineered polypeptide of any one of embodiments 136-143, wherein said epitope further comprises an activatable epitope selectively available for binding in a tumor microenvironment.
145. The engineered polypeptide of embodiment 144, wherein said activatable epitope is flanked by at least two copies of pH-sensitive helices.
146. The engineered polypeptide of any one of embodiments 136-145, wherein said epitope capable of binding: (i) said antibody or (ii) said peptide hormone or growth factor is derived from DLL3, PSMA, SSTR2, or any combination thereof.
147. The engineered polypeptide of embodiment 146, wherein said epitope is derived from DLL3, wherein said epitope comprises about 15 to about 260 contiguous residues of an extracellular domain of DLL3 having at least 80% identity to SEQ ID NO: 5, or a variant thereof.
148. The engineered polypeptide of embodiment 146 or 147, wherein said epitope comprises a sequence having at least 80% sequence identity to any one of SEQ ID NOs: 4, 5, 19, 20, 64, 65, or a variant thereof.
149. The engineered polypeptide of any one of embodiments 136-148, wherein said epitope is capable of binding: (i) said antibody or (ii) said peptide hormone or growth factor with a Kd of less than or equal to 20 nM, 15 nM, 10 nM, 9 nM, 8 nM, 7 nM, 6 nM, 5 nM, 4 nM, 3 nM, 2 nM, or 1 nM.
150. The engineered polypeptide of any one of embodiments 136-149, wherein said extracellularly-oriented polypeptide spacer domain comprises a hinge sequence from CD8, CD8a, CD8b, IgG4, IgG1, IgG2, IgG3, IgK, CD4, or CD28, or any combination thereof.
151. The engineered polypeptide of embodiment 150, wherein said extracellularly-oriented polypeptide spacer domain comprises a hinge sequence having at least 80% sequence identity to a hinge sequence of any one of SEQ ID NOs: 56, 57, 58, 59, 60, 61, 62, 66, 67, or a variant thereof.
152. The engineered polypeptide of any one of embodiments 136-151, wherein said transmembrane domain or said membrane affinity domain comprises a transmembrane domain.
153. The engineered polypeptide of embodiment 152, wherein said transmembrane domain comprises a single-pass transmembrane domain.
154. The engineered polypeptide of embodiment 152 or 153, wherein said transmembrane domain comprises a transmembrane domain derived from DLL3, PSMA, SSTR2, PD-L1, EGFR, CD28, CD4, CD8, CD8a, CD8b, ICOS, or CD73.
155. The engineered polypeptide of any one of embodiments 152-154, wherein said transmembrane domain comprises a sequence having at least 80% sequence identity to a transmembrane domain of any one of SEQ ID NOs: 68-76.
156. The engineered polypeptide of any one of embodiments 136-151, wherein said transmembrane domain or said membrane affinity domain comprises a membrane affinity domain.
157. The engineered polypeptide of embodiment 156, wherein said membrane affinity domain comprises a C1, C2, PH, FYVE, PX, or ENTH domain, or a transmembrane helix derived from bacteriorhodopsin.
158. The engineered polypeptide of embodiment 156 or 157, wherein said membrane affinity domain comprises a sequence having at least 80% sequence identity to any one of SEQ ID NOs: 77-82.
159. The engineered polypeptide of any one of embodiments 136-158, wherein said engineered polypeptide further comprises a scaffold domain: (i) N-terminal to said hinge domain and C-terminal to said epitope; or (ii) N-terminal to said hinge domain and containing said epitope.
160. The engineered polypeptide of embodiment 159, wherein said engineered polypeptide further comprises a scaffold domain N-terminal to said hinge domain and C-terminal to said epitope.
161. The engineered polypeptide of any one of embodiments 159-160, wherein said scaffold comprises a sequence having at least 80% sequence identity to SEQ ID NO: 63, or a variant thereof
162. The engineered polypeptide of embodiment 159 or 161, wherein said engineered polypeptide further comprises a scaffold domain N-terminal to said hinge domain and containing said epitope.
163. The engineered polypeptide of any one of embodiments 159-162, wherein said scaffold comprises a heavy chain variable (VH) domain and does not comprise a light chain variable (VL) domain.
164. The engineered polypeptide of embodiment 163, wherein said VH domain comprises an inactivating mutation in a CDR1, CDR2, or CDR3 region of said VH domain.
165. The engineered polypeptide of embodiment 163, wherein said scaffold comprises said epitope inserted in a CDR1, CDR2, or CDR3 region of said VH domain.
166. A nucleic acid comprising an ORF encoding the polypeptide of any one of embodiments 136-165, or a functional fragment thereof.
167. The nucleic acid of embodiment 166, further comprising a promoter operably linked to said ORF.
168. The nucleic acid of embodiment 167, wherein said promoter is not a T-cell specific promoter or a TCRA, TCRB, CMV, EF-1, hPGK, CD3, or RPBSA promoter.
169. The nucleic acid of any one of embodiments 167-168, wherein said promoter is a cancer-specific promoter.
170. The nucleic acid of any one of embodiments 167-169, wherein said promoter is a promoter of a gene overexpressed in a cancer cell versus a normal cell, or a functional fragment thereof.
171. The nucleic acid of any one of embodiments 167-170, wherein said promoter comprises a Survivin promoter (BIRC5), a CXCR4 promoter, an ATP binding cassette subfamily C member 4 (ABCC4) promoter, an anterior gradient 2, protein disulphide isomerase family member (AGR2) promoter, activation induced cytidine deaminase (AICDA) promoter, an UDP-GlcNAc:betaGal beta-1,3-N-acetylglucosaminyltransferase 3 (B3GNT3) promoter, a cadherin 3 (CDH3) promoter, a CEA cell adhesion molecule 5 (CEACAM5) promoter, a centromere protein F (CENPF) promoter, a centrosomal protein 55 (CEP55) promoter, a claudin 3 (CLDN3) promoter, a claudin 4 (CLDN4) promoter, a collagen type XI alpha 1 chain (COL11A1) promoter, a collagen type I alpha 1 chain (COL1A1) promoter, a cystatin SN (CST1) promoter, a denticleless E3 ubiquitin protein ligase homolog (DTL) promoter, a family with sequence similarity 111 member B (FAM111B) promoter, a forkhead box A1 (FOXA1) promoter, a kinesin family member 20A (KIF20A), a laminin subunit gamma 2 (LAMC2) promoter, a mitotic spindle positioning (MISP) promoter, a matrix metallopeptidase 1 (MMP1) promoter, a matrix metallopeptidase 12 (MMP12) promoter, a matrix metallopeptidase 13 (MMP13) promoter, a mesothelin (MSLN) promoter, a cell surface associated mucin 1 (MUC1) promoter, a phospholipase A2 group IID (PLA2G2D) promoter, a regulator of G protein signaling 13 (RGS13) promoter, a secretoglobin family 2A member 1 (SCGB2A1) promoter, topoisomerase II alpha (TOP2A) promoter, a ubiquitin D (UBD) promoter, a ubiquitin conjugating enzyme E2 C (UBE2C), a USH1 protein network component harmonin (USH1C), a V-set domain containing T cell activation inhibitor 1 (VTCN1) promoter, a Hexokinase type II promoter, a TRPM4 promoter, a stromelysin 3 promoter, a surfactant protein A promoter, a secretory leukoprotease inhibitor promoter, a tyrosinase promoter, a stress-inducible grp78/BiP promoter, an interleukin-10 promoter, an α-B-crystallin/heat shock protein 27 promoter, an epidermal growth factor receptor promoter, a mucin-like glycoprotein promoter, an mts1 promoter, an NSE promoter, a somatostatin receptor promoter, a c-erbB-3 promoter, a c-erbB-2 promoter, a c-erbB4 promoter, a thyroglobulin promoter, an α-fetoprotein promoter, a villin promoter, an albumin promoter, a glycoprotein A33 promoter, the B cell-specific Moloney leukemia virus insertion site 1 promoter, a cyclooxygenase-2 promoter, a fibroblast growth factor promoter; a human epidermal growth factor receptor 2, a human telomerase reverse transcriptase promoter; a kinase domain insert containing receptor promoter; a rad51 recombinase promoter; TTF-1, an urokinase-type plasminogen activator receptor promoter, a ubiquitin conjugating enzyme E2 T (UBE2T) promoter, a checkpoint kinase 1 (CHEK1) promoter, an epithelial cell transforming 2 promoter (ECT2), a BCL2-like 12 (BCL2L12) promoter, a centromere protein I (CENPI) promoter, an E2F transcription factor 1 (E2F1) promoter, a flavin adenine dinucleotide synthetase 1 (FLAD1) promoter, a protein phosphatase, Mg2+/Mn2+ dependent 1G (PPM1G) promoter, an ubiquitin conjugating enzyme E2 S (UBE2S) promoter, an aurora kinase A and ninein interacting protein (AUNIP) promoter, a cell division cycle 6 (CDC6) promoter, a centromere protein L (CENPL) promoter, a DNA replication helicase/nuclease 2 (DNA2) promoter, a DSN1 homolog, MIS12 kinetochore complex component (DSN1) promoter, a deoxythymidylate kinase (DTYMK) promoter, a G protein regulated inducer of neurite outgrowth 1 (GPRIN1) promoter, a mitochondrial fission regulator 2 (MTFR2) promoter, a RAD51 associated protein 1 (RAD51AP1) promoter, a small nuclear ribonucleoprotein polypeptide A′ (SNRPA1) promoter, an ATPase family, AAA domain containing 2 (ATAD2) promoter, a BUB1 mitotic checkpoint serine/threonine kinase (BUB1) promoter, a calcyclin binding protein (CACYBP) promoter, a cell division cycle associated 3 (CDCA3) promoter, a centromere protein O (CENPO) promoter, a flap structure-specific endonuclease 1 (FEN1) promoter, a forkhead box M1 (FOXM1) promoter, a cell proliferation regulating inhibitor of protein phosphatase 2A (KIAA1524) promoter, a kinesin family member 2C (KIF2C) promoter, a karyopherin subunit alpha 2 (KPNA2) promoter, a MYB proto-oncogene like 2 (MYBL2) promoter, a NIMA related kinase 2 (NEK2) promoter, a RAN binding protein 1 (RANBP1) promoter, a small nuclear ribonucleoprotein polypeptides B and B1 (SNRPB) promoter, a SPC24/NDC80 kinetochore complex component (SPC24) promoter, a transforming acidic coiled-coil containing protein 3 (TACC3) promoter, a TBC1 domain family member 31 (TBC1D31) promoter, a thymidine kinase 1 (TK1) promoter, a zinc finger protein 695 (ZNF695) promoter, an aurora kinase A (AURKA) promoter, a BLM RecQ like helicase (BLM) promoter, a chromosome 17 open reading frame 53 (C17orf53) promoter, a chromobox 3 (CBX30) promoter, a cyclin B1 (CCNB1) promoter, a cyclin E1 (CCNE1) promoter, a cyclin F (CCNF), a cell division cycle 20 (CDC20) promoter, a cell division cycle 45 (CDC45) promoter, a cell division cycle associated 5 (CDCA5) promoter, a cyclin dependent kinase inhibitor 3 (CDKN3) promoter, a cadherin EGF LAG seven-pass G-type receptor 3 (CELSR3) promoter, a centromere protein A (CENPA) promoter, a centrosomal protein 72 (CEP72) promoter, a CDC28 protein kinase regulatory subunit 2 (CKS2) promoter, a collagen type X alpha 1 chain (COL10A1) promoter, a chromosome segregation 1 like (CSE1L) promoter, a DBF4 zinc finger promoter, a GINS complex subunit 1 (GINS1) promoter, a G protein-coupled receptor 19 (GPR19) promoter, a kinesin family member 18A (KIF18A) promoter, a kinesin family member 4A (KIF4A) promoter, a kinesin family member C1 (KIFC1) promoter, a minichromosome maintenance 10 replication initiation factor (MCM10) promoter, a minichromosome maintenance complex component 2 (MCM2) promoter, a minichromosome maintenance complex component 7 (MCM7) promoter, a MRG domain binding protein (MRGBP) promoter, a methylenetetrahydrofolate dehydrogenase (NADP+ dependent) 2, methenyltetrahydrofolate cyclohydrolase (MTHFD2) promoter, a non-SMC condensin I complex subunit H (NCAPH) promoter, a kinetochore complex component (NDC80) promoter, a nudix hydrolase 1 (NUDT1) promoter, a ribonuclease H2 subunit A (RNASEH2A) promoter, a RuvB like AAA ATPase 1 (RUVBL1) promoter, a serologically defined breast cancer antigen NY-BR-85 (SGOL1) promoter, a SHC binding and spindle associated 1 (SHCBP1) promoter, a small nuclear ribonucleoprotein polypeptide G (SNRPG) promoter, a timeless circadian regulator promoter, a thyroid hormone receptor interactor 13 (TRIP13) promoter, a trophinin associated protein (TROAP) promoter, a ubiquitin conjugating enzyme E2 C (UBE2C) promoter, a WD repeat and HMG-box DNA binding protein 1 (WDHD1) promoter, an alpha fetoprotein (AFP) promoter, a functional fragment thereof, any combination thereof, a chimeric promoter compiled from a plurality of elements from the aforementioned, or a fully synthetic promoter composed of tiled transcription-factor binding sites derived from any of the aforementioned.
172. A vector comprising the nucleic acid of any one of embodiments 166-171.
173. The vector of embodiment 172, wherein said vector is a recombinant viral vector.
174. The vector of embodiment 172, wherein said vector is a non-viral vector 175. A method of detecting, imaging, or treating a cancer cell, comprising:
   (a) administering to a subject a composition comprising the nucleic acid of any one of embodiments 166-171 or the vector of any one of embodiments 172-174;
   (b) administering to said subject an antibody or antigen-binding fragment thereof, a protein ligand or functional fragment thereof, or a small molecule configured to bind said epitope.
176. The method of embodiment 175, wherein said administering to said subject said composition comprising said nucleic acid in (a) and said administering to said subject said antibody or antigen-binding fragment thereof in (b) are separated by at least about 8, 12, 16, 24, 36, 48, 60, 72, 84, or 96 hours.
177. The method of embodiment 175, wherein said composition or said antibody or antigen-binding fragment thereof, a protein ligand or functional fragment thereof, or a small molecule configured to bind said epitope are administered to said subject by parenteral, intramuscular, subcutaneous, intratumoral, rectal, vaginal, transdermal, or intravenous administration, or by cannula.
178. The method of any one of embodiments 175-177, wherein said composition comprising said nucleic acid or said vector is configured for intravenous administration.
179. The method of any one of embodiments 175-178, further comprising detecting binding of said antibody or antigen binding fragment, protein ligand or functional fragment thereof, or small molecule to said epitope.
180. The method of any one of embodiments 175-179, wherein said antibody or antigen-binding fragment thereof further comprises a radioisotope or a magnetic resonance imaging (MRI) contrast agent, or is chelated to a radioisotope or an MRI contrast agent.
181. The method of embodiment 180, wherein said antibody or antigen-binding fragment thereof further comprises a radioisotope or is chelated to a radioisotope.
182. The method of any one of embodiments 180-181, wherein said radioisotope comprises a positron-emitting radioisotope, an alpha-emitting radioisotope, a beta-emitting radioisotope, or a gamma-emitting radioisotope.
183. The method of any one of embodiments 180-182, wherein said radioisotope is a positron-emitting radioisotope and comprises ¹²⁴I, ⁶⁸Ga ¹¹C, ¹³N, ¹⁵O, ¹⁸F, ⁶⁸Ga, ⁶⁴Cu, ⁵²Mn, ⁵⁵Co, ⁸⁹Zr, ⁸²Rb, or any combination thereof.
184. The method of any one of embodiments 180-182, wherein said radioisotope comprises an alpha-emitting radioisotope and comprises ²²⁵Ac, ²¹¹At, ²²⁷Th, ²²⁴Ra, or any combination thereof.
185. The method of any one of embodiments 180-182, wherein said radioisotope comprises a beta-emitting radioisotope and comprises ¹⁷⁷Lu, ⁶⁷Cu, ¹³¹I, ⁹⁰Y, ⁸⁹Sr, ¹⁸⁶Re, ¹⁶⁵Dy, ³²P, ¹⁶⁶Ho, ¹⁸⁸Re, or any combination thereof.
186. The method of any one of embodiments 180-182, wherein said radioisotope is a gamma-emitting radioisotope and comprises ^{99m}Tc, ¹²³I, or ¹³¹I.
187. The method of embodiment 180, wherein said antibody or antigen-binding fragment thereof further comprises an MRI contrast agent, or is chelated to an MRI contrast agent.
188. The method of embodiment 187, wherein said MRI contrast agent comprises an iron oxide nanoparticle (IONP), a superparamagnetic iron platinum nanoparticle, manganese (II), or gadolinium (III).
189. The method of any one of embodiments 175-179, wherein said antibody or antigen-binding fragment thereof further comprises an antibody drug conjugate (ADC).
190. The method of embodiment 189, wherein said antibody drug conjugate is conjugated to a pyrrolobenzodiazepine (PBD), a protein toxin, diphtheria toxin, glucagon-like peptide (GLP-1), a cytotoxic immunomodulatory protein, a Fas ligand, auristatin or an analog thereof, a maytansinoid, a calicheamicin, duocarmycin or an analog thereof, or doxorubicin or an analog thereof.
191. The method of any one of embodiments 179-190, further comprising detecting said binding of said antibody or antigen binding fragment, protein ligand or functional fragment thereof, or small molecule to said epitope via MRI imaging, PET imaging, SPECT imaging, photoacoustic imaging or luminescence imaging performed on said subject.
192. The method of embodiment 191, wherein said epitope is displayed on a tumor cell of said subject.
193. The method of embodiment 192, wherein said tumor cell is a liver, ovarian, pancreatic, breast, lung, smooth muscle, bladder, kidney, skin, prostate, or bone tumor cell.
194. A cell comprising the nucleic acid of any one of embodiments 166-171, the vector of any one of embodiments 172-174, or the polypeptide of any one of embodiments 136-165.
195. A pharmaceutically-acceptable composition comprising the nucleic acid of any one of embodiments 166-171 or the vector of any one of embodiments 172-174.
196. A method of converting a nucleic acid encoding an epitope that specifically interacts with an antibody into a nucleic acid encoding a polypeptide comprising said epitope that is capable of display on a cell surface, comprising:
   (a) ligating said nucleic acid encoding said epitope that specifically interacts with said antibody to a nucleic acid encoding:
      (i) a hinge domain, and
      (ii) a transmembrane domain or a membrane affinity domain capable of associating with an outer membrane of a cell, or
   (b) synthesizing a nucleic acid wherein a nucleic acid encoding said epitope that specifically interacts with said antibody is linked to a nucleic acid encoding:
      (i) a hinge domain, and
      (ii) a transmembrane domain or a membrane affinity domain capable of associating with an outer membrane of a cell, wherein at least two of: said epitope that specifically interacts with an antibody; said hinge domain, and said transmembrane domain or a membrane affinity domain capable of associating with an outer membrane of a cell are heterologous to each other.
197. The method of embodiment 196, wherein said polypeptide capable of display on a cell surface does not display intracellular signaling activity associated with a protein from which said epitope, said hinge domain, or said transmembrane domain or affinity domain are derived.
198. The method of any one of embodiments 196-197, wherein said nucleic acid encoding: (i) said hinge domain, and (ii) said transmembrane domain or said membrane affinity domain capable of associating with said outer membrane of said cell does not encode an scFv.
199. The method of any one of embodiments 196-198, wherein said nucleic acid encoding: (i) said hinge domain, and (ii) said transmembrane domain or said membrane affinity domain capable of associating with said outer membrane of said cell does not encode a VL domain.
200. The method of any one of embodiments 196-199, wherein said nucleic acid encoding: (i) said hinge domain, and (ii) said transmembrane domain or said membrane affinity domain capable of associating with said outer membrane of said cell does not encode an intracellular signaling domain.
201. The method of any one of embodiments 196-199, wherein said nucleic acid encoding: (i) said hinge domain, and (ii) said transmembrane domain or said membrane affinity domain capable of associating with said outer membrane of said cell does not comprise an intracellular portion of a CD3zeta, CD137, or CD28 polypeptide.
202. The method of any one of embodiments 196-201, wherein an intracellularly-oriented portion of said polypeptide comprises less than or equal to 100, 75, 50, 25, 10, or 5 residues.
203. The method of any one of embodiments 196-202, wherein said epitope is derived from DLL3, PSMA, SSTR2, or any combination thereof.
204. The method of any one of embodiments 196-203, wherein said hinge domain is derived from CD8, CD8a, CD8b, IgG4, IgG1, IgG2, IgG3, IgK, CD4, CD28, or any combination thereof.
205. The method of any one of embodiments 196-204, wherein said transmembrane domain or said membrane affinity domain comprises a transmembrane domain.
206. The method of embodiment 205, wherein said transmembrane domain comprises a transmembrane domain derived from DLL3, PSMA, SSTR2, PD-L1, EGFR, CD28, CD4, CD8, CD8a, CD8b, ICOS, or CD73.
207. The method of any one of embodiments 196-204, wherein said transmembrane domain or said membrane affinity domain comprises a membrane affinity domain.
208. The method of embodiment 207, wherein said membrane affinity domain comprises a C1, C2, PH, FYVE, PX, or ENTH domain, or a transmembrane helix derived from bacteriorhodopsin.
209. A composition, comprising the nucleic acid of any one of embodiments 59-84, 111-132, or 166-171 or the vector of any one of embodiments 85-88, 133-135, or 172-174 and a pharmaceutically acceptable carrier.
210. The composition of embodiment 209, further comprising a transfection agent.

1-60. (canceled)

61. A method, comprising:
administering to a subject a composition comprising a nucleic acid encoding an engineered polypeptide, wherein said engineered polypeptide comprises:
(a) an extracellularly-oriented domain comprising an epitope capable of binding: (i) an antibody; or (ii) a peptide hormone or growth factor; wherein said extracellularly-oriented domain does not comprise an scFv; and
(b) a transmembrane domain or membrane affinity domain capable of associating with an outer membrane of a cell, comprising a transmembrane domain or membrane affinity domain from DLL3, PSMA, SSTR2, PD-L1, EGFR, CD28, CD4, ICOS, or CD73,
wherein (a) and (b) are heterologous to each other.

62. The method of claim 61, wherein said engineered polypeptide does not comprise a light chain variable (VL) domain.

63. The method of claim 61, wherein said engineered polypeptide does not comprise an intracellular portion of a CD3zeta, CD137, or CD28 polypeptide.

64. The method of claim 61, wherein said epitope capable of binding: (i) said antibody or (ii) said peptide hormone or growth factor is derived from DLL3, PSMA, SSTR2, or any combination thereof.

65. The method of claim 64, wherein said epitope is derived from DLL3.

66. The method of claim 65, wherein said epitope comprises about 15 to about 260 contiguous residues of an extracellular domain of DLL3 having at least 80% identity to SEQ ID NO: 5, or a variant thereof.

67. The method of claim 64, wherein said epitope comprises a sequence having at least 80% sequence identity to any one of SEQ ID NOs: 4, 5, 19, 20, 64, 65, or a variant thereof.

68. The method of claim 61, wherein said transmembrane domain or said membrane affinity domain capable of associated with said outer membrane of said cell comprises a sequence having at least 80% sequence identity to a transmembrane domain of any one of SEQ ID NOs: 68-76.

69. The method of claim 61, wherein said nucleic acid further comprises: (i) an open reading frame (ORF) encoding said engineered polypeptide; (ii) and a promoter operably linked to said ORF.

70. The method of claim 69, wherein said promoter is: (i) not a T-cell specific promoter; or (ii) not a TCRA, TCRB, CMV, EF-1, hPGK, CD3, or RPBSA promoter.

71. The method of claim 69, wherein said promoter is a promoter of a gene overexpressed in a cancer cell versus a normal cell, or a functional fragment thereof.

72. The method of claim 61, wherein said nucleic acid further comprises a vector encoding said engineered polypeptide, wherein said vector comprises a nanoplasmid vector, a close-ended linear duplex (CELiD) vector, or a Doggybone DNA vector (dbDNA).

73. The method of claim 72, wherein said vector comprises a nanoplasmid vector.

74. The method of claim 73, wherein a nanoplasmid vector comprises a minimized bacterial Co1E1 or R6K origin of replication and a bacterial RNA-based selectable marker.

75. The method of claim 61, further comprising administering to said subject an antibody or antigen-binding fragment thereof, a protein ligand or functional fragment thereof, or a small molecule configured to bind said epitope.

76. The method of claim 75, wherein said antibody or antigen-binding fragment thereof, said protein ligand or said functional fragment thereof, or said small molecule configured to bind said epitope is administered intravenously.

77. The method of claim 75, wherein: (i) said administering to said subject said composition comprising said nucleic acid; and (ii) said administering to said subject said antibody or antigen-binding fragment, said protein ligand or functional fragment thereof, or said small molecule configured to bind said epitope are separated by 96 hours or less.

78. The method of claim 75, further comprising detecting binding of said antibody or antigen binding fragment, said protein ligand or functional fragment thereof, or said small molecule to said epitope.

79. The method of claim 78, further comprising detecting binding of said antibody or antigen binding fragment, said protein ligand or functional fragment thereof, or said small molecule to said epitope via a non-invasive imaging method.

80. The method of claim 79, wherein said non-invasive imaging method is performed 120 hours or less after administering to said subject said antibody or antigen-binding fragment, said protein ligand or functional fragment thereof, or said small molecule configured to bind said epitope.

81. The method of claim 75, wherein said antibody or antigen binding fragment thereof or said protein ligand or functional fragment thereof further comprises a radioisotope.

82. The method of claim 81, wherein said radioisotope is a positron-emitting radioisotope.

83. The method of claim 79, wherein said non-invasive imaging method comprises positron emission tomography (PET).

84. The method of claim 75, wherein said antibody or antigen binding fragment thereof or said protein ligand or functional fragment thereof further comprises a magnetic resonance imaging (MRI) contrast agent.

85. The method of claim 84, wherein said non-invasive imaging method comprises MRI.

86. The method of claim 75, wherein said antibody or antigen binding fragment thereof or said protein ligand or functional fragment thereof comprises an antibody or antigen binding fragment thereof.

87. The method of claim 86, wherein said antibody or antigen binding fragment thereof comprises an antibody drug conjugate (ADC).

88. The method of claim 61, wherein said engineered polypeptide comprises an extracellularly-oriented polypeptide spacer domain of about 15 to about 40 angstroms in length when folded between (a) and (b)

89. The method of claim 88, wherein said extracellularly-oriented polypeptide spacer domain comprises a hinge sequence from IgG4, IgG1, IgG2, IgG3, IgK, CD4, or CD28, or any combination thereof.

90. The method of claim 88, wherein said extracellularly-oriented polypeptide spacer domain comprises a hinge sequence having at least 80% sequence identity to a hinge sequence of any one of SEQ ID NOs: 58, 59, 60, 61, 62, 66, 67, or a variant thereof.


SEQUENCE LISTING (94 sequences)

SEQ ID NO: 1 - protein, 76 aa, synthetic construct
GSEYEIRKALEELKASTAELKRATASLRAITEELKKNPSEDALVEHNRAIVEHNAIIVENNRIIAAVLELIVRAIK

SEQ ID NO: 2 - protein, 76 aa, synthetic construct
GSEYEIRKALEELKASTAELKRATASLRASTEELKKNPSEDALVENNRLIVEHNAIIVENNRIIAAVLELIVRAIK

SEQ ID NO: 3 - protein, 76 aa, synthetic construct
GSEYEIRKALEELKAATAELKRATASLRAITEELKKNPSEDALVEHNRAIVEHNAIIVEHNRIIAAVLELIVRAIK

SEQ ID NO: 4 - protein, 276 aa, Homo sapiens
DLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAF

SEQ ID NO: 5 - protein, 466 aa, Homo sapiens
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYL

SEQ ID NO: 6 - protein, 21 aa, Homo sapiens
RLCRPRSAPSRCGPGLRPCAP

SEQ ID NO: 7 - protein, 615 aa, synthetic construct
  VARIANT 1..76: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 83..158: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 165..240: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 247..615: note = This region may encompass one of the following sequences: DLTSNAVLTFIYFVVCIIGLCGNTLVIYVILRYAKMKTITNIYILNL AIADELFMLGLPFLAMQVALVHWPFGKAICRVVMTVDGINQFTSIFCLTVMSIDRYLA VVHPIKSAKWRRPRTAKMITMAVWGVSLLVILPIMIYAGLRSNQWGRSSCTINWPGES GAWYTGFIIYTFILGFLVPLTIICLCYLFIIIKVKSSGIRVGSSKRKKSEKKVTRMVS IVVAVFIFCWLPFYIFNVSSVSMAISPTPALKGMFDFVVVLTYANSCANPILYAF or MDMADEPLNGSHTWLSIPFDLNGSVVSTNTSNQTEPYYDLTSNAVLTFIYFVVCIIGL CGNTLVIYVILRYAKMKTITNIYILNLAIADELFMLGLPFLAMQVALVHWPFGKAICR VVMTVDGINQFTSIFCLTVMSIDRYLAVVHPIKSAKWRRPRTAKMITMAVWGVSLLVI LPIMIYAGLRSNQWGRSSCTINWPGESGAWYTGFIIYTFILGFLVPLTIICLCYLFII IKVKSSGIRVGSSKRKKSEKKVTRMVSIVVAVFIFCWLPFYIFNVSSVSMAISPTPAL KGMFDFVVVLTYANSCANPILYAFLSDNFKKSFQNVLCLVKVSGTDDGERSDSKQDKS RLNETTETQRTLLNGDLQTSI
XXXXXXXXXXXXXXXXXXXXXXXXXXXXXXXXXXXXXXXXXXXXXXXXXXXXXXXXXXXXXXXXXXXXXXXXXXXXGGSGGSXXXXXXXXXXXXXXXXXXXXXXXXXXXXXXXXXXXXXXXXXXXXXXXXXXXXXXXXXXXXXXXXXXXXXXXXXXXXGGSGGSXXXXXXXXXXXXXXXXXXXXXXXXXXXXXXXXXXXXXXXXXXXXXXXXXXXXXXXXXXXXXXXXXXXXXXXXXXXXGGSGGSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 8 - protein, 629 aa, synthetic construct
  VARIANT 1..76: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 97..172: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 179..254: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 261..629: note = This region may encompass one of the following sequences: DLTSNAVLTFIYFVVCIIGLCGNTLVIYVILRYAKMKTITNIYILNL AIADELFMLGLPFLAMQVALVHWPFGKAICRVVMTVDGINQFTSIFCLTVMSIDRYLA VVHPIKSAKWRRPRTAKMITMAVWGVSLLVILPIMIYAGLRSNQWGRSSCTINWPGES GAWYTGFIIYTFILGFLVPLTIICLCYLFIIIKVKSSGIRVGSSKRKKSEKKVTRMVS IVVAVFIFCWLPFYIFNVSSVSMAISPTPALKGMFDFVVVLTYANSCANPILYAF or MDMADEPLNGSHTWLSIPFDLNGSVVSTNTSNQTEPYYDLTSNAVLTFIYFVVCIIGL CGNTLVIYVILRYAKMKTITNIYILNLAIADELFMLGLPFLAMQVALVHWPFGKAICR VVMTVDGINQFTSIFCLTVMSIDRYLAVVHPIKSAKWRRPRTAKMITMAVWGVSLLVI LPIMIYAGLRSNQWGRSSCTINWPGESGAWYTGFIIYTFILGFLVPLTIICLCYLFII IKVKSSGIRVGSSKRKKSEKKVTRMVSIVVAVFIFCWLPFYIFNVSSVSMAISPTPAL KGMFDFVVVLTYANSCANPILYAFLSDNFKKSFQNVLCLVKVSGTDDGERSDSKQDKS RLNETTETQRTLLNGDLQTSI
XXXXXXXXXXXXXXXXXXXXXXXXXXXXXXXXXXXXXXXXXXXXXXXXXXXXXXXXXXXXXXXXXXXXXXXXXXXXGGSGGSFCYWKTCTGGSGGSXXXXXXXXXXXXXXXXXXXXXXXXXXXXXXXXXXXXXXXXXXXXXXXXXXXXXXXXXXXXXXXXXXXXXXXXXXXXGGSGGSXXXXXXXXXXXXXXXXXXXXXXXXXXXXXXXXXXXXXXXXXXXXXXXXXXXXXXXXXXXXXXXXXXXXXXXXXXXXGGSGGSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 9 - protein, 532 aa, synthetic construct
  VARIANT 1..76: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 98..173: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
  VARIANT 180..255: note = This region may encompass one of the following sequences: GSEYEIRKALEELKASTAELKRATASLRAITEELKKNPSEDALVEHN RAIVEHNAIIVENNRIIAAVLELIVRAIK or GSEYEIRKALEELKASTAELKRATA SLRASTEELKKNPSEDALVENNRLIVEHNAIIVENNRIIAAVLELIVRAIK or GSEYEIRKALEELKAATAELKRATASLRAITEELKKNPSEDALVEHNRAIVEHNAIIV EHNRIIAAVLELIVRAIK
XXXXXXXXXXXXXXXXXXXXXXXXXXXXXXXXXXXXXXXXXXXXXXXXXXXXXXXXXXXXXXXXXXXXXXXXXXXXRLCRPRSAPSRCGPGLRPCAPXXXXXXXXXXXXXXXXXXXXXXXXXXXXXXXXXXXXXXXXXXXXXXXXXXXXXXXXXXXXXXXXXXXXXXXXXXXXGGSGGSXXXXXXXXXXXXXXXXXXXXXXXXXXXXXXXXXXXXXXXXXXXXXXXXXXXXXXXXXXXXXXXXXXXXXXXXXXXXAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYL

SEQ ID NO: 10 - protein, 36 aa, synthetic construct
AAEQNPIYWARYADWLFTTPLLLLDLALLVDADEGT

SEQ ID NO: 11 - protein, 27 aa, synthetic construct
ACDDQNPWRAYLDLLFPTDTLLLDLLW

SEQ ID NO: 12 - protein, 27 aa, synthetic construct
ACDDQNPWRAYLKLLFPTKTLLLKLLW

SEQ ID NO: 13 - protein, 25 aa, synthetic construct
ACDDQNPWARYLDWLFPTDTLLLDL

SEQ ID NO: 14 - protein, 25 aa, synthetic construct
ACEEQNPWARYLEWLFPTETLLLEL

SEQ ID NO: 15 - protein, 32 aa, synthetic construct
ACEDQNPYWARYADWLFTTPLLLLDLALLVDG

SEQ ID NO: 16 - protein, 35 aa, synthetic construct
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 3..12: note = This region may encompass 1-10 residues
  VARIANT 13: note = Any amino acid
AXXXXXXXXXXXXPWRAYLDLLFPTDTLLLDLLWA

SEQ ID NO: 17 - protein, 47 aa, synthetic construct
GGTTTPAPRPPTPAPTIASQPLSLRPEACRPAAGGAVHTRGLDFACD

SEQ ID NO: 18 - protein, 228 aa, synthetic construct
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVST

SEQ ID NO: 19 - protein, 154 aa, synthetic construct
EPPAVGTACTRLCRPRSAPSRCGPGLRPCAPLEDECEAPLVCRAGCSPEHGFCEQPGECRCLEGWTGPLCTVPVSTGGEPPAVGTACTRLCRPRSAPSRCGPGLRPCAPLEDECEAPLVCRAGCSPEHGFCEQPGECRCLEGWTGPLCTVPVST

SEQ ID NO: 20 - protein, 207 aa, synthetic construct
QVQLVESGGGLVQPGGSLRLSCAASGGSEGSGETFSLGWFRQAPGQGLEAVAAIASMGGLTYYADSVKGRFTISRDNSKNTLYLQMNSLRAEDTAVYYCAAVGDVFAELPSSHNFRYWGQGTLVTVSSRGREPPAVGTACTRLCRPRSAPSRCGPGLRPCAPLEDECEAPLVCRAGCSPEHGFCEQPGECRCLEGWTGPLCTVPVST

SEQ ID NO: 21 - protein, 332 aa, synthetic construct
METDTLLLWVLLLWVPGSTGDAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDAAEQNPIYWARYADWLFTTPLLLLDLALLVDADEGT

SEQ ID NO: 22 - protein, 323 aa, synthetic construct
METDTLLLWVLLLWVPGSTGDAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDACDDQNPWRAYLDLLFPTDTLLLDLLW

SEQ ID NO: 23 - protein, 323 aa, synthetic construct
METDTLLLWVLLLWVPGSTGDAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDACDDQNPWRAYLKLLFPTKTLLLKLLW

SEQ ID NO: 24 - protein, 321 aa, synthetic construct
METDTLLLWVLLLWVPGSTGDAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDACDDQNPWARYLDWLFPTDTLLLDL

SEQ ID NO: 25 - protein, 321 aa, synthetic construct
METDTLLLWVLLLWVPGSTGDAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDACEEQNPWARYLEWLFPTETLLLEL

SEQ ID NO: 26 - protein, 328 aa, synthetic construct
METDTLLLWVLLLWVPGSTGDAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDACEDQNPYWARYADWLFTTPLLLLDLALLVDG

SEQ ID NO: 27 - protein, 222 aa, synthetic construct
MALPVTALLLPLALLLHAARPEPPAVGTACTRLCRPRSAPSRCGPGLRPCAPLEDECEAPLVCRAGCSPEHGFCEQPGECRCLEGWTGPLCTVPVSTGGEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACD

SEQ ID NO: 28 - protein, 275 aa, synthetic construct
MALPVTALLLPLALLLHAARPQVQLVESGGGLVQPGGSLRLSCAASGGSEGSGETFSLGWFRQAPGQGLEAVAAIASMGGLTYYADSVKGRFTISRDNSKNTLYLQMNSLRAEDTAVYYCAAVGDVFAELPSSHNFRYWGQGTLVTVSSRGREPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACD

SEQ ID NO: 29 - protein, 333 aa, synthetic construct
METDTLLLWVLLLWVPGSTGDMSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNEHLVYIMADKQKNGTKAIFQVHHNIEDGSVQLADHYQQNTPIGDGPVLLPDNHYLHTQSALSKDPNEKRDHMVLLEFVTAAGITHGMDELYKGGTTTPAPRPPTPAPTIASQPLSLRPEACRPAAGGAVHTRGLDFACDACDDQNPWRAYLKLLFPTKTLLLKLLW

SEQ ID NO: 30 - protein, 333 aa, synthetic construct
METDTLLLWVLLLWVPGSTGDMSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNEHLVYIMADKQKNGTKAIFQVHHNIEDGSVQLADHYQQNTPIGDGPVLLPDNHYLHTQSALSKDPNEKRDHMVLLEFVTAAGITHGMDELYKGGTTTPAPRPPTPAPTIASQPLSLRPEACRPAAGGAVHTRGLDFACDACDDQNPWRAYLDLLFPTDTLLLDLLW

SEQ ID NO: 31 - protein, 342 aa, synthetic construct
METDTLLLWVLLLWVPGSTGDMSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNEHLVYIMADKQKNGTKAIFQVHHNIEDGSVQLADHYQQNTPIGDGPVLLPDNHYLHTQSALSKDPNEKRDHMVLLEFVTAAGITHGMDELYKGGTTTPAPRPPTPAPTIASQPLSLRPEACRPAAGGAVHTRGLDFACDAAEQNPIYWARYADWLFTTPLLLLDLALLVDADEGT

SEQ ID NO: 32 - protein, 21 aa, Homo sapiens
METDTLLLWVLLLWVPGSTGD

SEQ ID NO: 33 - protein, 21 aa, Homo sapiens
MALPVTALLLPLALLLHAARP

SEQ ID NO: 34 - protein, 552 aa, synthetic construct
MRPSGTAGAALLALLAALCPASRAAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLSIATGMVGALLLLLVVALGIGLFMRRRHIVRKHMGGGSGGGSAYPYDVPDYAAYPYDVPDYA

SEQ ID NO: 35 - protein, 546 aa, synthetic construct
MRIFAVFIFMTYWHLLNAAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPLAHPPNERTHLVILGAILLCLGVALTFIFRHMGGGSGGGSAYPYDVPDYAAYPYDVPDYA

SEQ ID NO: 36 - protein, 591 aa, synthetic construct
MALPVTALLLPLALLLHAARPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 37 - protein, 353 aa, synthetic construct
MALPVTALLLPLALLLHAARPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 38 - protein, 531 aa, synthetic construct
MLLLVTSLLLCELPHPAFLLIPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFHMGGGSGGGSAYPYDVPDYAAYPYDVPDYA

SEQ ID NO: 39 - protein, 521 aa, synthetic construct
MCPRAARAPATLLLALGAVLWPAAGAAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLGGSSTGSHCHGSFSLIFLSLWAVIFVLYQ

SEQ ID NO: 40 - protein, 201 aa, synthetic construct
MALPVTALLLPLALLLHAARPEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 41 - protein, 279 aa, synthetic construct
MALPVTALLLPLALLLHAARPEPPAVGTACTRLCRPRSAPSRCGPGLRPCAPLEDECEAPLVCRAGCSPEHGFCEQPGECRCLEGWTGPLCTVPVSTGGEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 42 - protein, 386 aa, synthetic construct
MALPVTALLLPLALLLHAARPAGVFELQIHSFGPGPGPGAPRSPCSARLPCRLFFRVCLKPGLSEEAAESPCALGAALSARGPVYTGGCRPRSAPSRCGPGLRPCGGAPDLPLPDGLLQVPFRDAWPGTFSFIIETWREELGGGCRPRSAPSRCGPGLRPCGGGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 43 - protein, 332 aa, synthetic construct
MALPVTALLLPLALLLHAARPQVQLVESGGGLVQPGGSLRLSCAASGGSEGSGETFSLGWFRQAPGQGLEAVAAIASMGGLTYYADSVKGRFTISRDNSKNTLYLQMNSLRAEDTAVYYCAAVGDVFAELPSSHNFRYWGQGTLVTVSSRGREPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 44 - protein, 362 aa, synthetic construct
MALPVTALLLPLALLLHAARPQVQLVESGGGLVQPGGSLRLSCAASGGSGCRPRSAPSRCGPGLRPCGSGSLGWFRQAPGQGLEAVAAIASMGGLTYYADSVKGRFTISRDNSKNTLYLQMNSLRAEDTAVYYCAAVGGGCRPRSAPSRCGPGLRPCGGGPSSHNFRYWGQGTLVTVSSRGREPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 45 - protein, 290 aa, synthetic construct
MALPVTALLLPLALLLHAARPQVQLVESGGGLVQPGGSLRLSCAASGGSGCRPRSAPSRCGPGLRPCGSGSLGWFRQAPGQGLEAVAAIASMGGLTYYADSVKGRFTISRDNSKNTLYLQMNSLRAEDTAVYYCAAVGGGCRPRSAPSRCGPGLRPCGGGPSSHNFRYWGQGTLVTVSSRGRGGGSGGTTTPAPRPPTPAPTIASQPLSLRPEACRPAAGGAVHTRGLDFACDFWVLVVVGGVLACYSLLVTVAFIIFWVHMGGGSGGGSAYPYDVPDYAAYPYDVPDYA

SEQ ID NO: 46 - protein, 495 aa, synthetic construct
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLGGSSTGSHCHGSFSLIFLSLWAVIFVLYQ

SEQ ID NO: 47 - protein, 152 aa, synthetic construct
EPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHM

SEQ ID NO: 48 - protein, 230 aa, synthetic construct
EPPAVGTACTRLCRPRSAPSRCGPGLRPCAPLEDECEAPLVCRAGCSPEHGFCEQPGECRCLEGWTGPLCTVPVSTGGEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHM

SEQ ID NO: 49 - protein, 337 aa, synthetic construct
AGVFELQIHSFGPGPGPGAPRSPCSARLPCRLFFRVCLKPGLSEEAAESPCALGAALSARGPVYTGGCRPRSAPSRCGPGLRPCGGAPDLPLPDGLLQVPFRDAWPGTFSFIIETWREELGGGCRPRSAPSRCGPGLRPCGGGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHM

SEQ ID NO: 50 - protein, 283 aa, synthetic construct
QVQLVESGGGLVQPGGSLRLSCAASGGSEGSGETFSLGWFRQAPGQGLEAVAAIASMGGLTYYADSVKGRFTISRDNSKNTLYLQMNSLRAEDTAVYYCAAVGDVFAELPSSHNFRYWGQGTLVTVSSRGREPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHM

SEQ ID NO: 51 - protein, 313 aa, synthetic construct
QVQLVESGGGLVQPGGSLRLSCAASGGSGCRPRSAPSRCGPGLRPCGSGSLGWFRQAPGQGLEAVAAIASMGGLTYYADSVKGRFTISRDNSKNTLYLQMNSLRAEDTAVYYCAAVGGGCRPRSAPSRCGPGLRPCGGGPSSHNFRYWGQGTLVTVSSRGREPPAVGTACTRLCRPRSAPSRCGPGLRPCAPLEDECEAPLVCRAGCSPEHGFCEQPGECRCLEGWTGPLCTVPVSTGGTTTPAPRPPTPAPTIASQPLSLRPEACRPAAGGAVHTRGLDFACDFWVLVVVGGVLACYSLLVTVAFIIFWVHM

SEQ ID NO: 52 - protein, 241 aa, synthetic construct
QVQLVESGGGLVQPGGSLRLSCAASGGSGCRPRSAPSRCGPGLRPCGSGSLGWFRQAPGQGLEAVAAIASMGGLTYYADSVKGRFTISRDNSKNTLYLQMNSLRAEDTAVYYCAAVGGGCRPRSAPSRCGPGLRPCGGGPSSHNFRYWGQGTLVTVSSRGRGGGSGGTTTPAPRPPTPAPTIASQPLSLRPEACRPAAGGAVHTRGLDFACDFWVLVVVGGVLACYSLLVTVAFIIFWVHM

SEQ ID NO: 53 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 54 - protein, 744 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAE

SEQ ID NO: 55 - protein, 369 aa, Homo sapiens
MDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 56 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 57 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 58 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 223 aa, Homo sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 61 - protein, 226 aa, Homo sapiens
TPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 62 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 131 aa, Homo sapiens
QVQLVESGGGLVQPGGSLRLSCAASGGSEGSGETFSLGWFRQAPGQGLEAVAAIASMGGLTYYADSVKGRFTISRDNSKNTLYLQMNSLRAEDTAVYYCAAVGDVFAELPSSHNFRYWGQGTLVTVSSRGR

SEQ ID NO: 64 - protein, 76 aa, Homo sapiens
EPPAVGTACTRLCRPRSAPSRCGPGLRPCAPLEDECEAPLVCRAGCSPEHGFCEQPGECRCLEGWTGPLCTVPVST

SEQ ID NO: 65 - protein, 17 aa, Homo sapiens
CRPRSAPSRCGPGLRPC

SEQ ID NO: 66 - protein, 47 aa, Homo sapiens; identical to SEQ ID NO: 17

SEQ ID NO: 67 - protein, 229 aa, Homo sapiens; identical to SEQ ID NO: 62

SEQ ID NO: 68 - protein, 29 aa, Homo sapiens
LPPALGLLVAAGVAGAALLLVHVRRRGHS

SEQ ID NO: 69 - protein, 24 aa, Homo sapiens
WLCAGALVLAGGFFLLGFLFGWFI

SEQ ID NO: 70 - protein, 369 aa, Homo sapiens; identical to SEQ ID NO: 55

SEQ ID NO: 71 - protein, 21 aa, Homo sapiens
THLVILGAILLCLGVALTFIF

SEQ ID NO: 72 - protein, 23 aa, Homo sapiens
IATGMVGALLLLLVVALGIGLFM

SEQ ID NO: 73 - protein, 27 aa, Homo sapiens
FWVLVVVGGVLACYSLLVTVAFIIFWV

SEQ ID NO: 74 - protein, 22 aa, Homo sapiens
MALIVLGGVAGLLLFIGLGIFF

SEQ ID NO: 75 - protein, 21 aa, Homo sapiens
FWLPIGCAAFVVVCILGCILI

SEQ ID NO: 76 - protein, 26 aa, Homo sapiens
STGSHCHGSFSLIFLSLWAVIFVLYQ

SEQ ID NO: 77 - protein, 84 aa, Homo sapiens
GSSGSSGTTLASIGKDIIEDADGIAMPHQWLEGNLPVSAKCTVCDKTCGSVLRLQDWRCLWCKAMVHTSCKESLLTKCSGPSSG

SEQ ID NO: 78 - protein, 149 aa, Homo sapiens
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYR

SEQ ID NO: 79 - protein, 114 aa, Homo sapiens
GSSGSSGYGSEKKGYLLKKSDGIRKVWQRRKCSVKNGILTISHATSNRQPAKLNLLTCQVKPNAEDKKSFDLISHNRTYHFQAEDEQDYVAWISVLTNSKEEALTMAFSGPSSG

SEQ ID NO: 80 - protein, 90 aa, Homo sapiens
SMEGLVLGQKQPTWVPDSEAPNCMNCQVKFTFTKRRHHCRACGKVFCGVCCNRKCKLQYLEKEARVCVVCYETISKAQAFERMMSPTGSN

SEQ ID NO: 81 - protein, 145 aa, Homo sapiens
GPLGSMAAAPPLRDRLSFLHRLPILLKGTSDDDVPCPGYLFEEIAKISHESPGSSQCLLEYLLSRLHSSSGHGKLKVLKILLYLCSHGSSFFLLILKRNSAFIQEAAAFAGPPDPLHGNSLYQKVRAAAQDLGSTLFSDTVLPLA

SEQ ID NO: 82 - skipped

SEQ ID NO: 83 - protein, 33 aa, Homo sapiens
LPTTAQPTKKSTPKRRVCRLPRPETQKGPLCSP

SEQ ID NO: 84 - protein, 21 aa, Homo sapiens
IYIWAPLAGTCGVLLLSLVIT

SEQ ID NO: 85 - protein, 21 aa, Homo sapiens
ITLGLLVAGVLVLLVSLGVAI

SEQ ID NO: 86 - protein, 34 aa, Homo sapiens
PPPYLDNEKSNGTIIHVKGKHLCPSPLFPGPSKP

SEQ ID NO: 87 - protein, 36 aa, Homo sapiens
LNPEAGMWQCLLSDSGQVLLESNIKVLPTWSTPVQP

SEQ ID NO: 88 - protein, 8 aa, synthetic construct
FCYWKTCT

SEQ ID NO: 89 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 90 - protein, 68 aa, synthetic construct
  VARIANT 1..30: note = This region may encompass 1-10 GGS repeating units
  VARIANT 39..68: note = This region may encompass 1-10 GGS repeating units
GGSGGSGGSGGSGGSGGSGGSGGSGGSGGSFCYWKTCTGGSGGSGGSGGSGGSGGSGGSGGSGGSGGS

SEQ ID NO: 91 - protein, 21 aa, synthetic construct
RLCRPRSAPSRCGPGLRPCAP

SEQ ID NO: 92 - protein, 4 aa, synthetic construct
GGGS

SEQ ID NO: 93 - protein, 14 aa, synthetic construct
GGSFCYWKTCTGGS

SEQ ID NO: 94 - protein, 479 aa, synthetic construct
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFF